(19) United States
(12) Patent Application Publication
(10) Pub. No.: US 2005/0234096 A1
(43) Pub. Date: Oct. 20, 2005

(54) Tetrahydroisoquinoline derivatives

(76) Inventors: Hilmar Bischoff, Wuppertal (DE); Elke Dittrich-Wengenroth, Wuppertal (DE); Verena Vohringer, Wuppertal (DE); Heike Heckroth, Wuppertal (DE); Andrea Vaupel, Riehen (CH); Michael Woltering, Hilden (DE); Michael Otteneder, Arlesheim (CH)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/514,906

(22) PCT Filed: May 5, 2003
(86) PCT No.: PCT/EP03/04666

§ 371 Date: Jun. 9, 2005

(30) Foreign Application Priority Data
May 17, 2002 (DE) 10222034.4

(51) Int. Cl. C07D 217/22; A61K 31/47

(52) U.S. Cl. 514/309; 546/141

(57) ABSTRACT

The invention relates to novel substituted tetrahydroisoquinoline derivatives, methods for the production thereof and the use thereof in medicaments, especially as potent PPAR-delta activating compounds for the prophylaxis and/or treatment of cardiovascular diseases, especially dislipidaemia, coronary heart disease and arteriosclerosis.

The present application relates to novel substituted tetrahydroisoquinoline derivatives, to processes for their preparation and to their use in medicaments, especially as potent PPAR-delta-activating compounds for the prophylaxis and/or treatment of cardiovascular disorders, especially dyslipidemias, coronary heart diseases and arteriosclerosis.

In spite of many successful therapies, coronary heart diseases (CHDs) remain a serious public health problem. While treatment with statins, by inhibition of HMG-CoA reductase, very successfully lowers the plasma concentration of LDL cholesterol and this leads to a significant lowering in the mortality of patients at risk, there is to date a lack of successful treatment strategies for the therapy of patients having an unfavorable HDL/LDL cholesterol ratio and/or hypertriglyceridemia.

To date, fibrates constitute the only form of therapy for patients of these risk groups. They act as weak agonists of the peroxisome proliferator-activated receptor (PPAR)-alpha (Nature 1990, 347, 645-50). A disadvantage of fibrates which have been approved to date is that their interaction with the receptor is only weak and leads to high daily doses and distinct side effects.

For the peroxisome-proliferator-activated receptor (PPAR)-delta (Mol. Endocrinol. 1992, A 163441), the first pharmacological findings in animal models indicate that potent PPAR-delta agonists may likewise lead to an improvement in the HDL/LDL cholesterol ratio and in hypertriglyceridemia.

WO 00/23407 discloses PPAR modulators for the treatment of obesity, atherosclerosis and/or diabetes. WO 93/15051 and EP 636 608-A1 describe 1-benzenesulfonyl-1,3-dihydroindol-2-one derivatives having partial phenoxyacetic acid structure as vasopressin and/or oxytocin antagonists.

It is an object of the present invention to provide novel compounds which can be used as PPAR-delta modulators.

It has now been found that compounds of the general formula (I) 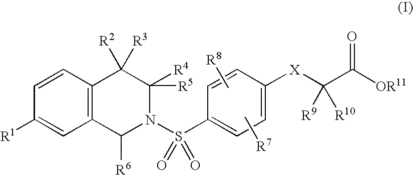
in which
X is O, S or CH₂,
R¹ is halogen, (C₁-C₆)-alkoxy, (C₂-C₆)-alkenyloxy, (C₃-C₇)-cycloalkoxy, optionally halogen-, (C₁-C₄)-alkyl-, trifluoromethyl- or (C₁-C₄)-alkoxy-substituted benzyloxy or
is (C₆-C₁₀)-aryl or 5- to 6-membered heteroaryl having up to three heteroatoms from the group of N, O and/or S, each of which may itself be mono- to trisubstituted, identically or differently, by substituents selected from the group of halogen, (C₁-C₆)-alkyl, (C₁-C₆)-alkoxy, trifluoromethyl, trifluoromethoxy, amino, mono- and di-(C₁-C₆)-alkylamino,
R² and R³ are the same or different and are each independently hydrogen or (C₁-C₆)-alkyl which is optionally substituted by phenyl, or, together with the carbon atom to which they are bonded, form a 3- to 7-membered, spiro-linked cycloalkyl ring,
R⁴ and R⁵ are the same or different and are each independently hydrogen or (C₁-C₆)-alkyl,
R⁶ is hydrogen or (C₁-C₆)-alkyl,
R⁷ is hydrogen or (C₁-C₆)-alkyl,
R⁸ is hydrogen, (C₁-C₆)-alkyl, (C₁-C₆)-alkoxy or halogen,
R⁹ and R¹⁰ are the same or different and are each independently hydrogen or (C₁-C₄)-alkyl, and
R¹¹ is hydrogen or is a hydrolyzable group which can be degraded to the corresponding carboxylic acid,
   and the pharmaceutically acceptable salts, solvates and solvates of the salts thereof exhibit pharmacological action and can be used as medicaments or for the preparation of medicament formulations.

In the context of the invention, in the definition of R¹¹, a hydrolyzable group means a group which, especially in the body, leads to conversion of the —C(O)OR¹¹ moiety to the corresponding carboxylic acid (R¹¹ hydrogen). Such groups are, for example and with preference: benzyl, (C₁-C₆)-alkyl or (C₃-C₈)-cycloalkyl, each of which is optionally mono- or polysubstituted, identically or differently, by halogen, hydroxyl, amino, (C₁-C₆)-alkoxy, carboxyl, (C₁-C₆)-alkoxycarbonyl, (C₁-C₆)-alkoxycarbonylamino or (C₁-C₆)-alkanoyloxy, or in particular (C₁-C₄)-alkyl which is optionally mono- or polysubstitated, identically or differently, by halogen, hydroxyl, amino, (C₁-C₄)-alkoxy, carboxyl, (C₁-C₄)-alkoxycarbonyl, (C₁-C₄)-alkoxycarbonylamino or (C₁-C₄)-alkanoyloxy.

In the context of the invention, (C₁-C₆)-alkyl and (C₁-C₄)-alkyl represent a straight-chain or branched allyl radical having from 1 to 6 and from 1 to 4 carbon atoms respectively. Preference is given to a straight-chain or branched alkyl radical having from 1 to 4 carbon atoms. Preferred examples, include: methyl, ethyl, n-propyl, isopropyl and tert-butyl.

In the context of the invention, (C₂-C₆)-alkenyl and (C₂-C₄)-alkenyl represent a straight-chain or branched alkenyl radical having from 2 to 6 and from 2 to 4 carbon atoms respectively. Preference is given to a straight-chain or branched alkenyl radical having from 2 to 4 carbon atoms. Preferred examples include: vinyl, allyl, ispropenyl and n-but-2-en-1-yl.

In the context of the invention, (C₃-C₈)-cycloalkyl represents a monocyclic cycloalkyl group having from 3 to 8 carbon atoms. Preferred examples include: cyclopropyl, cyclobutyl, cyclopentyl and cyclohexyl.

In the context of the invention, (C₆-C₁₀)-aryl represents an aromatic radical having preferably from 6 to 10 carbon atoms. Preferred aryl radicals are phenyl and naphthyl.

In the context of the invention, (C₁-C₆)-alkoxy and (C₁-C₄)-alkoxy represent a straight-chain or branched alkoxy radical having from 1 to 6 and from 1 to 4 carbon atoms respectively. Preference is given to a straight-chain or branched alkoxy radical having from 1 to 4 carbon atoms. Preferred examples include: methoxy, ethoxy, n-propoxy, isopropoxy and tert-butoxy.

In the context of the invention, (C₂-C₆)-alkenyloxy and (C₂-C₄)-alkenyloxy represent a straight-chain or branched alkenyloxy radical having from 2 to 6 and from 2 to 4 carbon atoms respectively. Preference is given to a straight-chain or branched alkenyloxy radical having from 2 to 4 carbon atoms. Preferred examples include: vinyloxy, allyloxy, isopropenyloxy and n-but-2-en-1-yloxy.

In the context of the invention, (C₃-C₇)-cycloalkoxy and (C₅-C₆)-cycloalkoxy represent a monocyclic cycloalkoxy group having from 3 to 7 and from 5 to 6 carbon atoms respectively. Preference is given to a cycloalkoxy radical having from 5 to 6 carbon atoms. Preferred examples include: cyclopropoxy, cyclobutoxy, cyclopentoxy and cyclohexoxy.

In the context of the invention, (C₁-C₆)-alkoxycarbonyl represents a straight-chain or branched alkoxy radical which has from 1 to 6 carbon atoms and is attached via a carbonyl group. Preference is given to a straight-chain or branched alkoxycarbonyl radical having from 1 to 4 carbon atoms. Preferred examples include: methoxycarbonyl, ethoxycarbonyl, n-propoxycarbonyl, isopropoxycarbonyl and tert-butoxycarbonyl.

In the context of the invention, (C₁-C₆)-alkoxy carbonylamino represents an amino group having a straight-chain or branched alkoxycarbonyl substituent which has from 1 to 6 carbon atoms in the alkoxy radical and is attached via the carbonyl group. Preference is given to an alkoxycarbonylamino radical having from 1 to 4 carbon atoms. Preferred examples include: methoxycarbonylamino, ethoxycarbonylamino, n-propoxycarbonylamino and tert-butoxycarbonylamino.

In the context of the invention, (C₁-C₆)-alkanoyloxy represents a straight-chain or branched alkyl radical which has from 1 to 6 carbon atoms and bears a double-bonded oxygen atom in the 1-position and is attached in the 1-position via a further oxygen atom. Preferred examples include: acetoxy, propionoxy, n-butyroxy, isobutyroxy, pivaloyloxy, n-hexanoyloxy.

In the context of the invention, mono-(C₁-C₆)-alkylamino and mono-(C₁-C₄)-alkylamino represent an amino group having a straight-chain or branched alkyl substituent which has from 1 to 6 and from 1 to 4 carbon atoms respectively. Preference is given to a straight-chain or branched monoalkylamino radical having from 1 to 4 carbon atoms. Preferred examples include: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents having in each case from 1 to 6 and from 1 to 4 carbon atoms respectively. Preference is given to straight-chain or branched dialkylamino radicals having in each case from 1 to 4 carbon atoms. Preferred examples include: N,N-dimethylamino, N,N-diethylamino, N-ethyl-N-methyl amino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, 5- or 6-membered heteroaryl having up to 3 identical or different heteroatoms from the group of S, N and/or O preferably represents an aromatic heterocycle which is attached via a ring carbon atom of the heteroaromatic or, if appropriate, via a ring nitrogen atom of the heteroaromatic. Examples include: furanyl, pyrrolyl, thienyl, thiazolyl, oxazolyl, imidazolyl, triazolyl, pyridyl, pyrimidinyl, pyridazinyl, pyrazinyl. Particular preference is given to pyridyl, pyrimidinyl, pyridazinyl and pyrazinyl.

In the context of the invention, halogen includes fluorine, chlorine, bromine and iodine. Preference is given to chlorine or fluorine.

Depending on the substitution pattern, the inventive compounds can exist in stereoisomeric forms which either behave like image and mirror image (enantiomers) or do not behave like image and mirror image (diastereomers). The invention relates both to the enantiomers or diastereomers and to their respective mixtures. The racemic forms, like the diastereomers, can be separated in a known manner into the stereoisomerically uniform constituents.

Furthermore, certain compounds can be present in tautomeric forms. This is known to those skilled in the art, and such compounds are likewise encompassed by the scope of the invention.

The compounds according to the invention can also be present as salts. In the context of the invention, preference is given to physiologically acceptable salts.

Physiologically acceptable salts can be salts of the inventive compounds with inorganic or organic acids. Preference is given to salts with inorganic acids, for example hydrochloric acid, hydrobromic acid, phosphoric acid or sulfuric acid, or to salts with organic; carboxylic or sulfonic acids, for example acetic acid, propionic acid, maleic acid, fumaric acid, malic acid, citric acid, tartaric acid, lactic acid, benzoic acid, or methanesulfonic acid, ethanesulfonic acid, benzenesulfonic acid, toluenesulfonic acid or naphthalenedisulfonic acid.

Physiologically acceptable salts can also be salts of the inventive compounds with bases, for example metal or ammonium salts. Preferred examples are alkali metal salts (e.g. sodium salts or potassium salts), alkaline earth metal salts (e.g. magnesium salts or calcium salts), and also ammonium salts which are derived from ammonia or organic amines, for example ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The inventive compounds can also be present in the form of their solvates, in particular in the form of their hydrates.

Preference is given to compounds of the general formula (I), in which
X is O or S,
R¹ is (C₂-C₄)-alkenyloxy, (C₅-C₆)-cycloalkoxy or is halogen or (C₁-C₄)-alkoxy, or
is 6-membered heteroaryl having up to two nitrogen atoms, or is phenyl or benzyloxy which may themselves each be mono- to disubstituted, identically or differently, by substituents selected from the group of fluorine, chlorine, (C₁-C₄)-alkyl, (C₁-C₄)-alkoxy, trifluoromethyl, trifluoromethoxy, amino, mono- and di-(C₁-C₄)-alkylamino,
R² and R³ are the same or different and are each independently hydrogen or are methyl or ethyl which may be substituted by phenyl, or, together with the carbon atom to which they are bonded, form a 4- to 6-membered, spiro-linked cycloalkyl ring,
R⁴ and R⁵ are each hydrogen,
R⁶ is hydrogen or methyl,
R⁷ is hydrogen or methyl,
R⁸ is hydrogen, (C₁-C₄)-alkyl, (C₁-C₄)-alkoxy, fluorine or chlorine,
R⁹ and R¹⁰ are the same or different and are each independently hydrogen or methyl, and
R¹¹ is hydrogen or (C₁-C₄)-alkyl.

Particular preference is given to compounds of the general formula (I) in which
X is O,
R¹ is pyridyl or is in particular phenyl which may itself in each case be mono- or disubstituted, identically or differently, by substituents selected from the group of fluorine, chlorine, methyl, methoxy, trifluoromethyl, trifluoromethoxy, amino and dimethylamino,
R² is hydrogen or methyl,
R³ is methyl or phenethyl, or
R² and R³, together with the carbon atom to which they are bonded, form a spiro-linked cyclopentane or cyclohexane ring,
R⁴ and R⁵ are each hydrogen,
R⁶ is hydrogen or methyl,
R⁷ is hydrogen or methyl,
R⁸ is methyl,
R⁹ and R¹⁰ are each hydrogen, and
R¹¹ is ethoxy or in particular is hydrogen.

The radical definitions listed above, in general or specified within areas of preference, apply both to the end products of the formula (I) and correspondingly to the starting materials and intermediates required for the preparation in each case.

The radical definitions specified individually in the particular combinations or preferred combinations of radicals, irrespective of the combinations of radicals specified in each case, are also replaced as desired by radical definitions of other combinations.

Of particular importance are compounds of the formula (I-A) 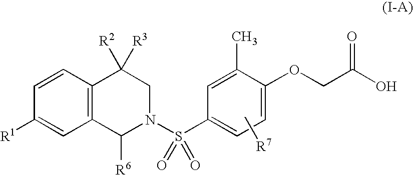
in which
R² is hydrogen,
R³ is methyl or phenethyl, or
R² and R³ are both methyl, or, together with the carbon atom to which they are bonded, form a spiro-linked cyclopentane or cyclohexane ring, and
R¹, R⁶ and R⁷ are each as defined above.

Moreover, a process has been found for preparing the inventive compounds of the general formula (I), characterized in that
[A] Compounds of the General Formula (II) 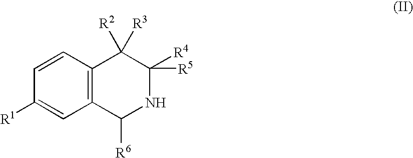
in which R¹, R², R³, R⁴, R⁵ and R⁶ are each as defined above
are converted initially, using a compound of the general formula (III) 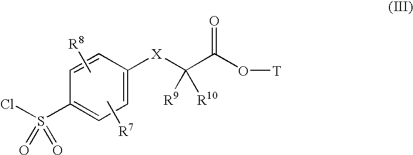
in which X, R⁷, R⁸, R⁹ and R¹⁰ are each as defied above and
T is benzyl or (C₁-C₆)-alkyl,
in an inert solvent in the presence of a base, to compounds of the general formula (I-B) 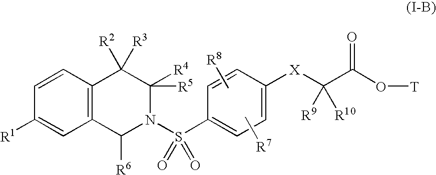
in which T, X, R¹, R², R³, R⁴, R⁵, R⁶, R⁷, R⁸, R⁹ and R¹⁰ are each as defined above,
these are then reacted with acids or bases or, in the case that T is benzyl, also hydrogenolytically, to give the corresponding carboxylic acids of the general formula (I-C) 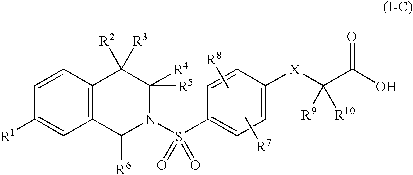
in which X, R¹, R², R³, R⁴, R⁵, R⁶, R⁷, R⁸, R⁹ and R¹⁰ are each as defined above,
and these carboxylic acids (I-C) are optionally modified further by known methods for esterification to give compounds of the general formula (I), or
   [B] Compounds of the General Formula (IV) 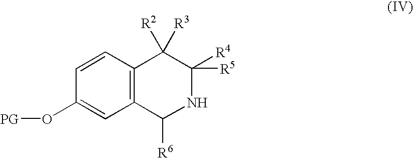
in which R², R³, R⁴, R⁵ and R⁶ are each as defined above and
PG is a suitable hydroxyl protecting group such as methyl or benzyl
is converted initially, using a compound of the general formula (III) in an inert solvent in the presence of a base to compounds of the general formula (V) 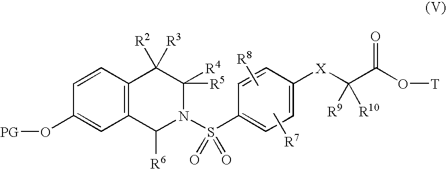
in which PG, T, X, R², R³, R⁴, R⁵, R⁶, R⁷, R⁸, R⁹ and R¹⁰ are each as defined above,
in the next reaction step the protecting group PG is removed by suitable methods, for example by treatment with boron tribromide (PG=methyl) or hydrogenolytically (PG=benzyl) to give compounds of the general formula (VI) 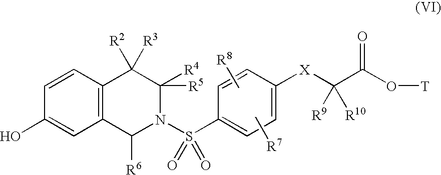
in which T, X, R², R³, R⁴, R⁵, R⁶, R⁷, R⁸, R⁹ and R¹⁰ are each as defined above,
and the compounds of the general formula (VI) are then either

[B-1] Reacted with a Compound of the General Formula (VII)
R¹²-Z (VII)
in which
R¹² is (C₁-C₆)-alkyl, (C₂-C₆)-alkenyl, (C₃-C₇)-cycloalkyl or optionally halogen-, (C₁-C₄)-alkyl-, trifluoromethyl- or (C₁-C₄)-alkoxy-substituted benzyl, and
Z is a suitable leaving group, for example halogen, mesylate or tosylate,
in an inert solvent in the presence of a base to give compounds of the general formula (I-D) 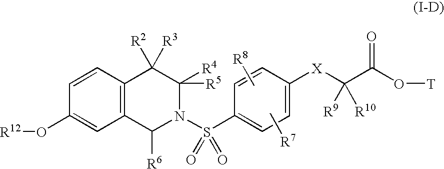
in which T, X, R², R³, R⁴, R⁵, R⁶, R⁷, R⁸, R⁹, R¹⁰ and R¹² are each as defined above,
and these are converted, using acids or bases or, in the case that T is benzyl, also hydrogenolytically, to the corresponding carboxylic acids of the general formula (I-E) 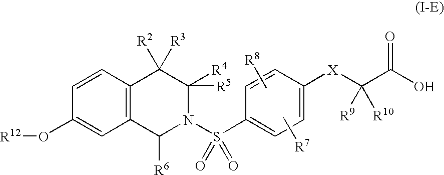
in which X, R², R³, R⁴, R⁵, R⁶, R⁷, R⁸, R⁹, R¹⁰ and R¹² are each as defined above, or

[B-2] Initially Converted, Using Trifluoromethanesulfonic Anhydride in the Presence of a Base, to Compounds of the General Formula (VIII) 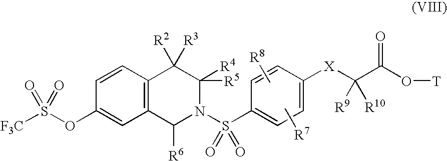
in which T, X, R², R³, R⁴, R⁵, R⁶, R⁷, R⁸, R⁹ and R¹⁰ are each as defined above,
and these are reacted in a coupling reaction with a compound of the general formula (IX) 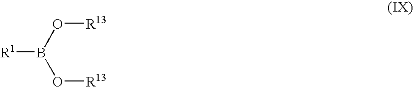
in which
R¹ is (C₆-C₁₀)-aryl or 5- to 6-membered heteroaryl having up to three heteroatoms from the group of N, O and/or S, each of which may itself be mono- to trisubstituted, identically or differently, by substituents selected from the group of halogen, (C₁-C₆)-alkyl, (C₁-C₆)-alkoxy, trifluoromethyl, trifluoromethoxy, amino, mono- and di-(C₁-C₆)-alkylamino, and
R¹³ is hydrogen or methyl or both radicals together form a CH₂CH₂ or C(CH₃)₂—C(CH₃)₂ bridge,
in an inert solvent in the presence of a suitable palladium catalyst and of a base to give compounds of the general formula (I-B) [cf., for example, W. Hahnfeld, M. Jung, Pharmazie 1994, 49, 18-20; idem, Liebigs Ann. Chem. 1994, 59-64].

Inert solvents for the process step (II)+(III)→(I-B) or (IV)+(III)→(V) are, for example, halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as nitromethane, ethyl acetate, acetone, dimethylformamide, dimethyl sulfoxide, acetonitrile, N-methylpyrrolidinone or pyridine. It is equally possible to use mixtures of the solvents mentioned. Preference is given to dichloromethane or tetrahydrofuran.

Suitable bases for the process step (II)+(III)→(I-B) or (IV)+(III)→(V) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal hydrides such as sodium hydride, or organic amines such as pyridine, triethylamine, ethyldiisopropylamine, N-methylmorpholine or N-methylpiperidine. Particular preference is given to amine bases such as triethylamine, pyridine or ethyldiisopropylamine; optionally in the presence of catalytic amounts (approx. 10 mol %) of 4-N,N-dimethylaminopyridine or 4-pyrrolidinopyridine.

The base is used in an amount of from 1 to 5 mol, preferably from 1 to 2.5 mol, based on 1 mole of the compound of the general formula (III).

The reaction is generally effected in a temperature range of from −20° C. to +100° C., preferably from 0° C. to +75° C. The reaction may be carried out at standard, elevated or at reduced pressure (e.g. from 0.5 to 5 bar). In general, standard pressure is used.

Inert solvents for the process step (I-B)→(I-C) are, for example, halohydrocarbons such as dichloromethane, 1,2-dichloroethane or trichloroethylene, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as nitromethane, acetone, dimethylformamide, dimethyl sulfoxide, acetonitrile or N-methylpyrrolidinone. It is equally possible to use mixtures of the solvents mentioned. Preference is given to alcohols such as methanol or ethanol.

Suitable bases for the process step (I-B)→(I-C) are the customary inorganic bases. They preferably include alkali metal hydroxides, for example lithium hydroxide, sodium hydroxide or potassium hydroxide, or alkali metal or alkaline earth metal carbonates such as sodium carbonate, potassium carbonate or calcium carbonate. Particular preference is given to lithium hydroxide or sodium hydroxide.

The bases used in an amount of from 1 to 5 mol, preferably from 1 to 3 mol, based on 1 mole of the compound of the general formula (I-B).

Suitable acids for the process step (I-B)→(I-C) are the customary inorganic acids, for example hydrochloric acid or sulfuric acid, or sulfonic acids such as toluenesulfonic acid, methanesulfonic acid or trifluoromethanesulfonic acid, or carboxylic acids such as trifluoroacetic acid.

The reaction is effected generally within a temperature range of from −20° C. to +100° C., preferably from 0° C. to +30° C. The reaction may be carried out at standard, elevated or at reduced pressure (e.g. from 0.5 to 5 bar). In general, standard pressure is used.

Inert solvents for the process step (V)+(VII)→(I-D) are, for example, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as nitromethane, acetone, dimethylformamide, dimethyl sulfoxide, acetonitrile or N-methylpyrrolidinone. It is equally possible to use mixtures of the solvents mentioned. Preference is given to dimethylformamide.

Suitable bases for the process step (VI)+(VI)→(I-D) are the customary inorganic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, or alkali metal hydrides such as sodium hydride or potassium hydride. Particular preference is given to sodium hydride or potassium carbonate.

The base is used in an amount of from 1 to 5 mol, preferably from 1 to 2 mol, based on 1 mole of the compound of the general formula (VI).

The reaction is effected generally within a temperature range of from −20° C. to +150° C., preferably from 0° C. to +100° C. The reaction may be carried out at standard, elevated or at reduced pressure (e.g. from 0.5 to 5 bar). In general, standard pressure is used.

Inert solvents for the process step (VI)→(VIII) are, for example, halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether hydrocarbons such as benzene, xylene, toluene, hexane, cyclohexane or mineral oil fractions, or other solvents such as nitromethane, acetone, dimethylformamide, dimethyl sulfoxide, acetonitrile or N-methylpyrrolidinone. It is equally possible to use mixtures of the solvents mentioned. Preference is given to tetrahydrofuran or dichloromethane.

Suitable bases for the process step (VI)→(VIII) are the customary inorganic or organic bases. These preferably include alkali metal or alkaline earth metal carbonates such as sodium carbonate, potassium carbonate or calcium carbonate, or organic amines such as pyridine, triethylamine, ethyldiisopropylamine, N-methylmorpholine or N-methylpiperidine. Particular preference is given to triethylamine or ethyldiisopropylamine, optionally in the presence of catalytic amounts (approx. 10 mol %) of 4-N,N-dimethylaminopyridine or 4-pyrrolidinopyridine.

The base is used in an amount of from 1 to 5 mol, preferably from 1 to 2.5 mol, based on 1 mole of the compound of the general formula (VI).

The reaction is effected generally within a temperature range of from −20° C. to +150° C., preferably from 0° C. to +70° C. The reaction may be carried out at standard, elevated or at reduced pressure (e.g. from 0.5 to 5 bar). In general, standard pressure is used.

Inert solvents for the process step (VIII)+(IX)→(I-B) are, for example, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as dimethylformamide, acetonitrile or else water. It is equally possible to use mixtures of the solvents mentioned. Preference is given to toluene, dimethylformamide or a mixture of dimethylformamide and water.

Suitable bases for the process step (VIII)+(IX)→(I-B) are the customary inorganic or organic bases. These preferably include alkali metal or alkaline earth metal carbonates such as sodium carbonate, potassium carbonate or calcium carbonate, alkali metal phosphates such as sodium phosphate or potassium phosphate, or organic amines such as triethylamine, ethyldiisopropylamine, N-methylmorpholine or N-methylpiperidine. Particular preference is given to sodium carbonate or potassium carbonate or potassium phosphate.

The base is used in an amount of from 1 to 5 mol, preferably from 1 to 2 mol, based on. 1 mole of the compound of the general formula (VIII).

The reaction is effected generally within a temperature range of from −20° C. to +150° C., preferably from 0° C. to +100° C. The reaction may be carried out at standard, elevated or at reduced pressure (e.g. from 0.5 to 5 bar). In general, standard pressure is used.

The compounds of the general formula (II) are known or can be prepared in analogy to literature processes, for example by reacting a compound of the formula (X) 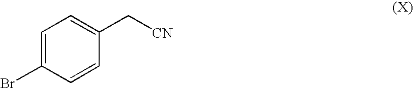
in an inert solvent in the presence of a base with an equivalent amount or an excess of a compound of the general formula (XI)
R^{2*}Y (XI)
in which
R^{2*} is (C₁-C₆)-alkyl which is optionally substituted by phenyl, and
Y is a suitable leaving group, for example halogen, mesylate or tosylate,
   or with a compound of the formula (XII
   Y—(CH₂)ₙ—Y (XII)
   in which Y is as defined above, and
n is 2, 3, 4, 5 or 6,
   to give a compound of the general formula (XIII) 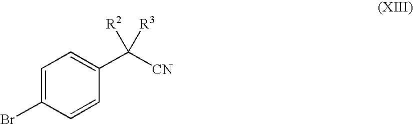
   in which R² and R³ are each as defined above but are not both simultaneously hydrogen,
   then reacting this
[a] in the case that R⁴ and R⁵ are both hydrogen, with the aid of a complex boro- or aluminohydride, for example lithium aluminum hydride or lithium borhydride, optionally in the presence of trimethylsilyl chloride, or
[b] in the case that R⁴ and R⁵ are each (C₁-C₆)-alkyl, in a one-pot reaction or in two steps, with an organometallic compound of the general formula (XIV)
   R^{4*}-M (XIV)
   in which
   R^{4*} is (C₁-C₆)-alkyl and
   M is Li, —Mg—Cl, —Mg—Br or —Mg—I,
      to give a compound of the general formula (XV) 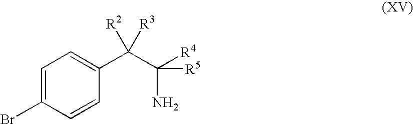
      in which R², R³, R⁴ and R⁵ are each as defined above,
      subsequently converting it using formic acid to a compound of the general formula (XVI) 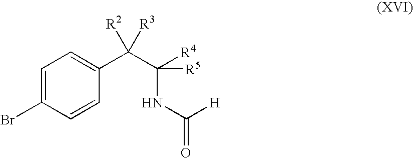
      then reacting it, in a coupling reaction with a compound of the general (formula (IX) in an inert solvent in the presence of a suitable palladium catalyst and of a base, to give a compound of the general formula (XVII) 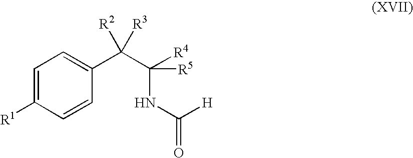
      in which R¹, R², R³, R⁴ and R⁵ are each as defined above,
      then reacting it in the presence of an acid with a compound of the general formula (XVIII)
      R⁶—CHO (XVIII)
      in which R⁶ is as defined above
      with cyclization to give a compound of the general formula (XIX) 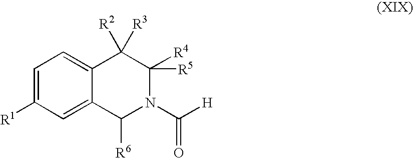
      in which R¹, R², R³, R⁴, R⁵ and R⁶ are each as defined above,
      and finally detaching the formyl group in (XIX) with the aid of a base [for the process step (XIII)→(XV), cf., for example, A. Giannis, K. Sandhoff, Angew. Chem. 1989, 101, 220-222; R. Armouroux, G. P. Axiotis, Synthesis 1981, 270-272; E. Ciganek, J. Org. Chem. 1992, 57, 4521-4527; A. Nakazato, T. Kumagai, K. Ohta, S. Chaki, S. Okuyama, K. Tomisawa, J. Med. Chem. 1999, 42, 3965-3970; E. F. J. de Vries, P. Steenwinkel, J. Brussee, C. G. Kruse, A. van der Gen, J. Org. Chem. 1993, 58, 4315-4325; M. Chastrette, G. P. Axiotis, Synthesis 1980, 889-890; for the process step (XVI)+(IX)→(XVII) cf, for example, W. Hahnfeld, M. Jung, Pharmazie 1994, 49, 18-20; idem, Liebigs Ann. Chem. 1994, 59-64; for the process step (XVII)+(XVIII)→(XIX) cf., for example, A. P. Venkov, I. I. Ivanov, Synth. Commun. 1993, 23, 1707-1719; B. E. Maryanoff, M. C. Rebarchak, Synthesis 1992, 12, 1245-1248].

Inert solvents for the process step (X)+(XI) or (XII)→(XIII) are, for example, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide, dimethyl sulfoxide or N-methylpyrrolidinone. It is equally possible to use mixtures of the solvents mentioned. Preference is given to a mixture of diethyl ether and dimethyl sulfoxide.

Suitable bases for the process step (X)+(XI) or (XII)→(XIII) are the customary inorganic or organic bases. These preferably include alkali metal hydroxides, for example lithium hydroxide, sodium hydroxide or potassium hydroxide, alkali metal hydrides such as sodium hydride or potassium hydride, or amides such as sodium amide, lithium bis(trimethylsilyl)amide or lithium diisopropylamide. Particular preference is given to potassium hydroxide.

The base is used in an amount of from 1 to 10 mol, preferably from 2 to 5 mol, based on 1 mole of the compound of the general formula (X).

The reaction is effected generally within a temperature range of from −20° C. to +100° C., preferably from 0° C. to +30° C. The reaction may be carried out at standard, elevated or at reduced pressure (e.g. from 0.5 to 5 bar). In general, standard pressure is used.

Inert solvents for the process step (XV)→(XVI) are, for example, halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions. It is equally possible to use mixtures of the solvents mentioned. Preference is given to xylene (with removal of the water formed in the reaction).

The formic acid is used in this reaction step in an amount of from 1 to 5 mol, preferably from 1 to 3 mol, based on 1 mole of the compound of the general formula (XV).

The reaction is effected generally within a temperature range of from 0° C. to +150° C., preferably from +20° C. to +130° C. The reaction may be carried out at standard, elevated or at reduced pressure (for example from 0.5 to 5 bar). In general, standard pressure is used.

Inert solvents for the process step (XVI)+(IX)→(XVII) are, for example, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or mineral oil fractions, or other solvents such as dimethylformamide, acetonitrile or else water. It is equally possible to use mixtures of the solvents mentioned. Preference is given to toluene, dimethylformamide or acetonitrile.

Suitable bases for the process step (XVI)+(IX)→(XVII) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal phosphates such as sodium phosphate or potassium phosphate, or organic amines such as pyridine, triethylamine, ethyldiisopropylamine, N-methylmorpholine or N-methylpiperidine. Particular preference is given to sodium carbonate or potassium carbonate or potassium phosphate.

The base is used in an amount of from 1 to 5 mol, preferably from 2 to 3 mol, based on 1 mole of the compound of the general formula (XVI).

The reaction is effected generally within a temperature range of from 0° C. to +150° C., preferably from +20° C. to +100° C. The reaction may be carried out at standard, elevated or at reduced pressure (e.g. from 0.5 to 5 bar). In general, standard pressure is used.

Inert solvents for the process step (XVII)+(XVIII)→(XIX) are, for example, halohydrocarbons such as dichloromethane, trichloromethane, tetrachloromethane, trichloroethane, tetrachloroethane, 1,2-dichloroethane or trichloroethylene, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions. It is equally possible to use mixtures of the solvents mentioned. It is also possible to carry out the reaction without solvent.

Suitable acids for the process step (XVI)+(XVIII)→(XIX) are the customary inorganic or organic acids. These preferably include hydrochloric acid, sulfuric acid or phosphoric acid, or carboxylic acids such as formic acid, acetic acid or trifluoroacetic acid, or sulfonic acids such as toluenesulfonic acid, methanesulfonic acid or trifluoromethanesulfonic acid. Particular preference is given to a mixture of acetic acid and trifluoroacetic acid which is used in a large excess and simultaneously serves as the solvent.

The reaction is effected generally within a temperature range of from −20° C. to +150° C., preferably from +20° C. to +120° C. The reaction may be carried out at standard, elevated or at reduced pressure (e.g. from 0.5 to 5 bar). In general, standard pressure is used.

The compounds of the general formula (III) are known or can be prepared in analogy to literature processes, for example, by converting a compound of the general formula (XX) 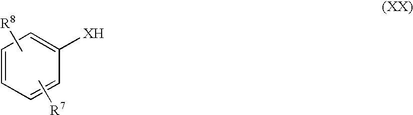
in which R⁷, R⁸ and X are each as defined above,
initially using a compound of the general formula (XXI) 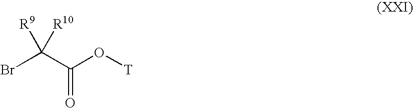
in which R⁹, R¹⁰ and T are each as defined above,
in an inert solvent in the presence of a base to a compound of the general formula (XXII) 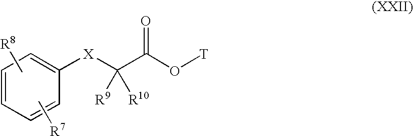
in which R⁷, R⁸, R⁹, R¹⁰, X and T are each as defined above,
and then reacting it with chlorosulfonic acid [cf., for example, P. D. Edwards, R. C. Mauger, K. M. Cottrell, F. X. Morris, K. K. Pine, M. A. Sylvester, C. W. Scott, S. T. Furlong, Bioorg. Med. Chem. Lett. 2000, 10, 2291-2294].

Inert solvents for the process step (XX)+(XXI)→(XXII) are, for example, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as acetone, dimethylformamide, dimethyl sulfoxide, acetonitrile or N-methylpyrrolidinone. It is equally possible to use mixtures of the solvents mentioned. Preference is given to dimethylformamide or acetone.

Suitable bases for the process step (XX)+(XXI)→(XXII) are the customary inorganic or organic bases. These preferably include alkali metal hydroxides, for example lithium hydroxide, sodium hydroxide or potassium hydroxide, alkali metal or alkaline earth metal carbonates such as sodium carbonate, potassium carbonate or calcium carbonate, alkali metal hydrides such as sodium hydride, or organic amines such as pyridine, triethylamine, ethyldiisopropylamine, N-methylmorpholine or N-methylpiperidine. Particular preference is given to potassium carbonate.

The base is used in an amount of from 1 to 5 mol, preferably from 1 to 2 mol, based on 1 mole of the compound of the general formula (XX).

The reaction is effected generally within a temperature range of from −20° C. to +150° C., preferably from 0° C. to +80° C. The reaction may be carried out at standard, elevated or at reduced pressure (e.g. from 0.5 to 5 bar). In general, standard pressure is used.

The compounds of the general formula (IV) may be prepared in analogy to literature processes, for example by converting a compound of the general formula (XXIII) 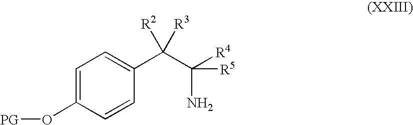
in which R², R³, R⁴, R⁵ and PG are each as defined above either
[a] in accordance with the above-described process steps (XV)→(XVI) and (XVI)+(XVIII)→(XIX) to a compound of the general formula (XXIV) 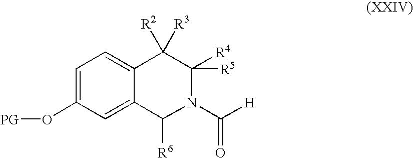
   in which R², R³, R⁴, R⁵, R⁶ and PG are each as defined above,
   and subsequently detaching the formyl group in (XXIV) with the aid of a base, or
[b] initially reacting it with a carboxylic acid or carboxylic acid derivative of the general formula (XXV) 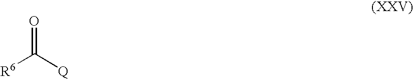
   in which R⁶ is as defined above and
   Q is hydroxyl, halogen or the complementary carboxylic anhydride radical
   to give a compound of the general formula (XXVI) 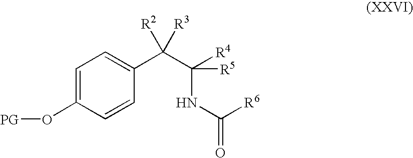
   in which R², R³, R⁴, R⁵, R⁶ and PG are each as defined above,
   subsequently cyclizing with the aid of a phosphorus chloride, for example phosphorus oxychloride, to give a compound of the general formula (XXVII) 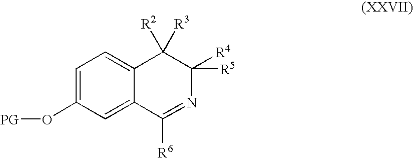
   and then reducing it with the aid of a complex boro- or aluminohydride, for example sodium borohydride [for the process step (XXVI)→(XXVI), cf., for example, E. Martinez, J. C. Estevez, R. J. Estevez, M. C. Villayerde, L. Castedo, Tetrahedron Lett. 1998, 39, 1231-1232].

The compounds of the general formula (XXIII) are obtainable analogously to the above-described process (X)+(XI) or (XII)→(XIII)→(XV) from compounds of the general formula (XXVIII)

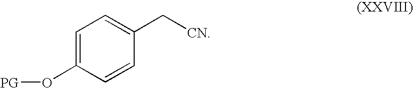

The compounds of the general formulae (VII), (IX), (X), (XI), (XII), (XVIII), (XX), (XXI), (XXV) and (XXVIII) are commercially obtainable, known from the literature or can be prepared in analogy to literature processes.

The process according to the invention can be illustrated by the following reaction schemes 1-4:

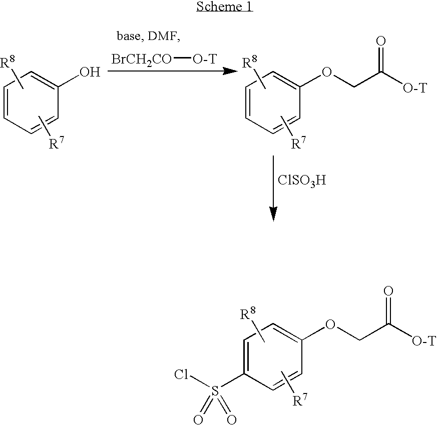

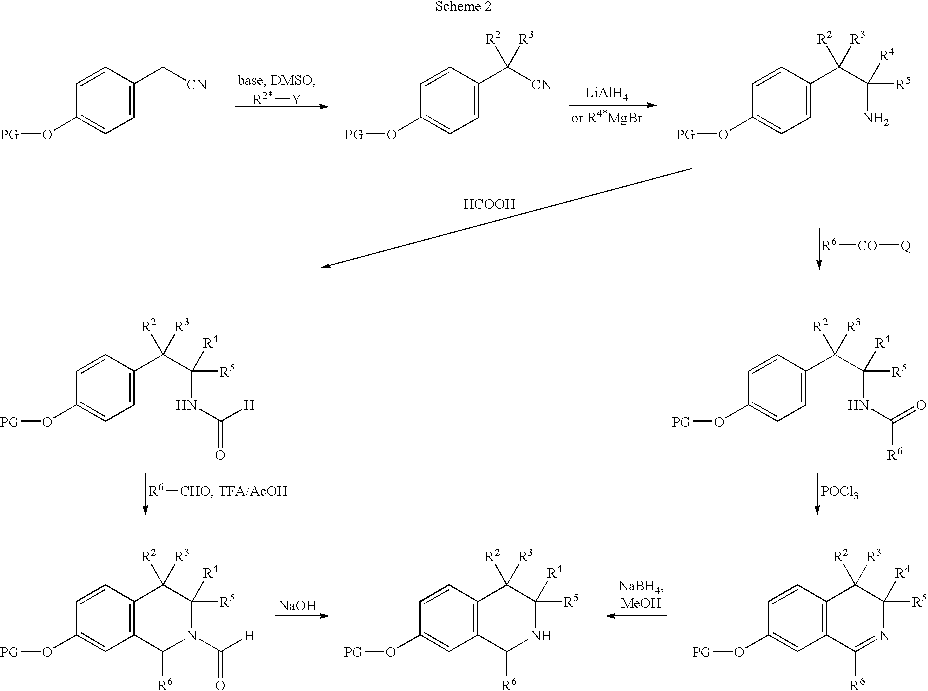

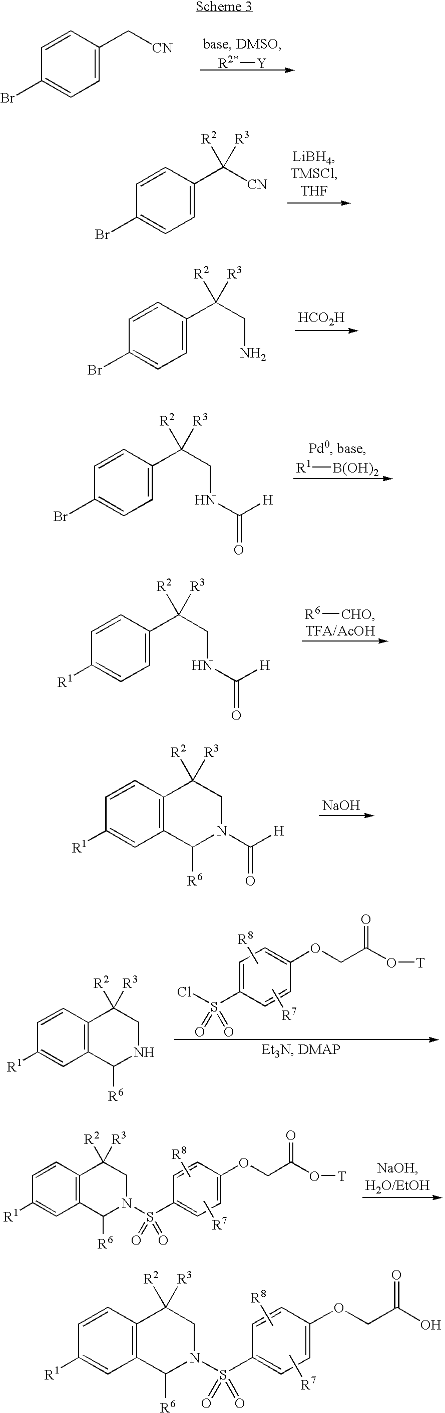

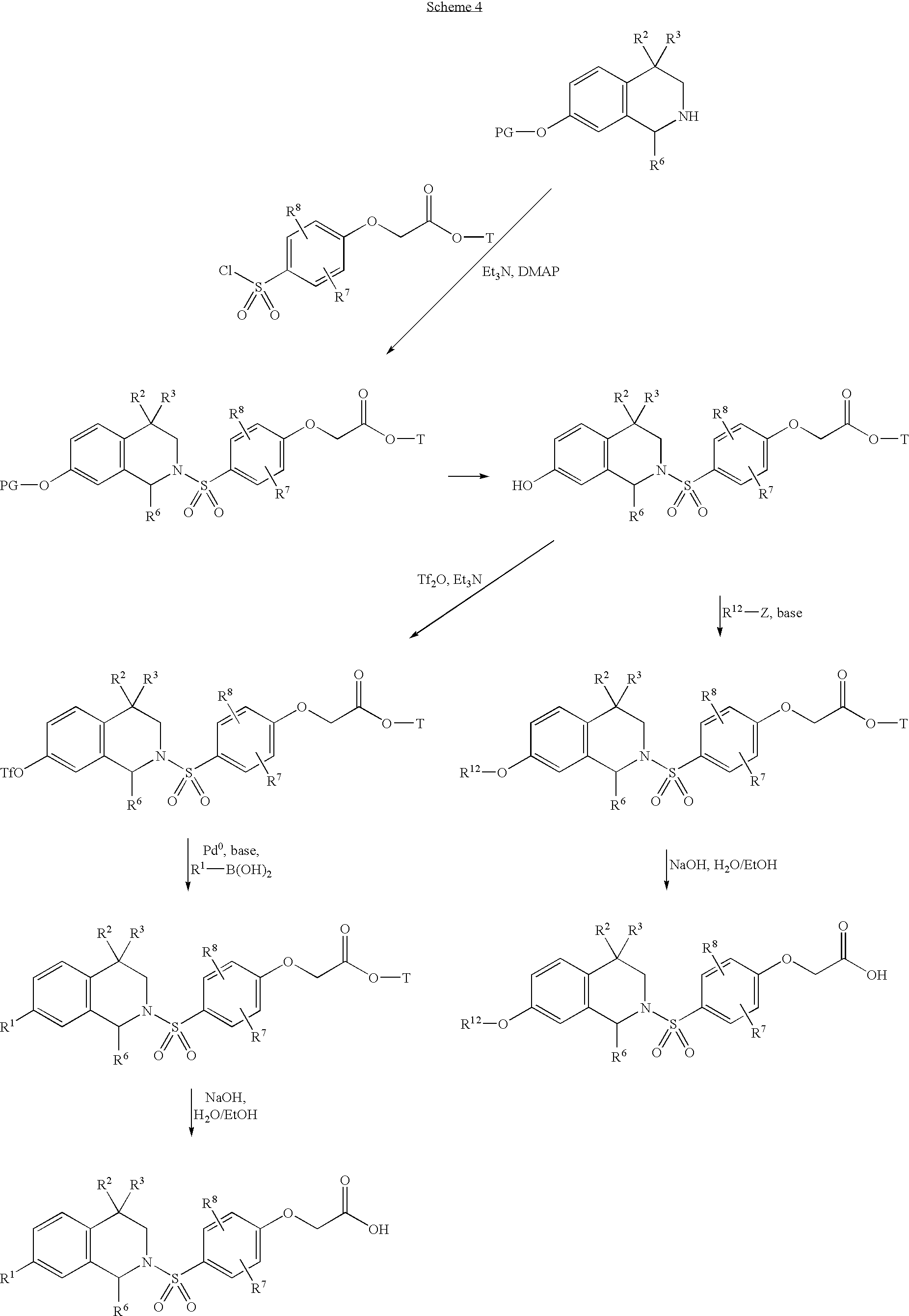

The inventive compounds of the formula (I) exhibit a surprising and valuable pharmacological spectrum of action and can therefore be used as versatile medicaments. In particular, they are suitable for the treatment of coronary heart disease, for the prophylaxis of myocardial infarction and for the treatment of restenosis after coronary angioplasty or stenting. The inventive compounds of the formula (I) are preferentially suitable for treating arteriosclerosis and hypercholesterolemia, for increasing pathologically low HDL levels and for lowering elevated triglyceride and LDL levels. In addition, they can be used for treating obesity, diabetes, for treating metabolic syndrome (glucose intolerance, hyperinsulinemia, dyslipidemia and hypertension owing to insulin resistance), hepatic fibrosis and cancer.

The novel active ingredients may be administered alone or, if required, in combination with other active ingredients, preferably from the group of CETP inhibitors, antidiabetics, antioxidants, cytostatics, calcium antagonists, antihypertensives; thyroid hormones and/or thyroid mimetics, inhibitors of HMG-CoA reductase, inhibitors of HMG-CoA reductase expression, squalene synthesis inhibitors, ACAT inhibitors, perfusion promoters, platelet aggregation inhibitors, anticoagulants, angiotensin II receptor antagonists, cholesterol: absorption inhibitors, MTP inhibitors, aldolase reductase inhibitors, fibrates, niacin, anoretics, lipase inhibitors and PPAR-α and/or PPAR-γ agonists.

The activity of the inventive compounds can be tested, for example, in vitro by the transactivation assay described in the experimental section.

The activity of the inventive compounds can be tested in vivo, for example, by investigations described in the experimental section.

Useful administration forms for the administration of the compounds of the general formula (I) are all customary administration forms, i.e. oral, parenteral, inhalative, nasal, sublingual, rectal, external, for example transdermal, or local, for example in the case of implants or stents. In the case of parenteral administration, mention should be made in particular of intravenous, intramuscular or subcutaneous administration, for example as a subcutaneous depot. Preference is given to oral or parenteral administration. Very particular preference is given to oral administration.

The active ingredients may be administered alone or in the form of preparations. Preparations suitable for oral administration include tablets, capsules, pellets, coated tablets, pills, granules, solid and liquid aerosols, syrups, emulsions, suspensions and solutions. In this case, the active ingredient has to be present in such an amount that a therapeutic action is achieved. In general, the active ingredient may be present in a concentration of from 0.1 to 100% by weight, in particular from 0.5 to 90% by weight, preferably from 5 to 80% by weight. In particular, the concentration of the active ingredient should be from 0.5 to 90% by weight, i.e. the active ingredient should be present in amounts which are sufficient to attain the dosage range specified.

For this purpose, the active ingredients may be converted to the customary preparations in a manner known per se. This is effected using inert, nontoxic, pharmaceutically suitable carriers, excipients, solvents, vehicles, emulsifiers and/or dispersants.

Examples of excipients include: water, nontoxic organic solvents, for example paraffins, vegetable oils (e.g. sesame oil), alcohols (e.g. ethanol, glycerol), glycols (e.g. polyethylene glycol), solid carriers such as natural or synthetic ground minerals (e.g. talc or silicates), sugars (e.g. lactose), emusifiers, dispersants (e.g. polyvinylpyrrolidone) and lubricants (e.g. magnesium sulfate).

In the case of oral administration, tablets may of course also comprise additives such as sodium citrate together with additives such as starch, gelatin and the like. Aqueous preparations for oral administration may also be admixed with flavor improvers or dyes.

In the case of oral administration, preference is given to administering dosages of from 0.001 to 5 mg/kg, preferably from 0.005 to 3 mg/kg, of bodyweight per 24 hours.

The working examples which follow illustrate the invention. The invention is not restricted to the examples.

Unless stated otherwise, the percentages in the tests and examples which follow are percentages by weight, parts are parts by weight. Solvent ratios, dilution ratios and concentration data of liquid/liquid mixtures are based in each case on the volume.

HPLC Method 1:

Instrument: HP 1100 with DAD detection; column: Kromasil RP-18, 60 mm×2 mm, 3.5 μm; eluent: A 5 ml HClO₄/l H₂O, B=ACN; gradient: 0 min 2% B, 0.5 min 2% B, 4.5 min 90% B, 6.5 min 90% B; flow rate: 0.75 ml/min; temp.: 30° C.; detection: UV 210 mm

HPLC Method 2:

Instrument: HP 1100 mit DAD detection; column: Kromasil RP-18, 60 mm×2 mm, 3.5 μm; eluent: A=5 ml HClO₄/l H₂O, B=ACN; gradient: 0 min 2% B, 0.5 min 2% B, 4.5 min 90% B, 9 min 90% B; flow rate: 0.75 ml/min; temp.: 30° C.; detection: UV 210 nm

LC-MS Method 3:

Column: symmetry C-18, 5 μm, 2.1×150 mm; eluent: A=acetonitrile, B=water+0.3 g of 30% HCl/l; gradient: 0.0 min 2% A 2.5 min 95% A→5 min 95% A; flow rate: 1.2 ml/min; temp.: 70° C.; detection: UV 210 nm

GC-MS Method 4:

GC instrument type: HP 6890; column: HP-5, 30 m×320 μm×0.25 μm (film thickness); injector temp.: 250° C.; oven temp.: 60° C.; gradient: 60° C., 1 min→16° C./min→300° C., 1 min; carrier gas: helium; constant flow rate: 1.5 ml/min; ionization: EI/CI positive.

GC-MS Method 5:

Instrument: Varian GC; column: HP-5, 30 m×320 μm×0.25 μm (film thickness); injector temp.: 250° C.; oven temp.: 60° C.; gradient: 60° C.→10° C./min→300° C., 6 min; carrier gas: helium; constant flow rate: 1.5 ml/min; ionization: EI/CI positive.

GC Method 6:

Instrument: HP 5890 Series 2; column: DB1, 30 m×0.25 mm, film thickness 0.25 μm; injector temp.: 250° C.; gradient: 50° C.→10° C./min→320° C., 3 min; detector: FID, temp.: 330° C.; injection volume: 1 μl.

### Abbreviations:

ACN acetonitrile
DCI direct chemical ionization (in MS)
DMF N,N-dimethylformamide
DMSO dimethyl sulfoxide
EI electron impact ionization (in MS)
ESI electrospray ionization (in MS)
GC gas chromatography
HPLC high-pressure, high-performance liquid chromatography
LC-MS liquid chromatography-coupled mass spectroscopy
MS mass spectroscopy
NMR nuclear magnetic resonance spectroscopy
R_{f} retention index (in TLC)
RP reverse phase (in HPLC)
THF tetrahydrofuran
   Starting Compounds:

### EXAMPLE 1A

### Ethyl Phenoxyacetate

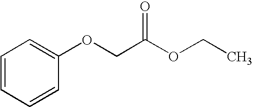

23.53 g (0.25 mol) of phenol and 34.55 g (0.25 mol) of potassium carbonate are suspended in 100 ml of acetone and heated to reflux for 1 hour. Subsequently, a solution of 43.84 g (0.26 mol) of ethyl bromoacetate in 100 ml of acetone is added dropwise and the mixture is heated to reflux overnight. After cooling to room temperature, the mixture is added to ice and the aqueous phase is extracted three times with methylene chloride. The combined organic phases are dried over sodium sulfate and freed of solvent under reduced pressure. 46 g (85% of theory) of the desired product is obtained in 87% purity.

GC-MS (method 4): Rₜ=7.44 min.

MS (ESIpos): 180 (M)⁺

¹H NMR (200 MHz, CDCl₃): δ=1.30 (t, 3H), 4.28 (q, 2H), 4.62 (s, 2H), 6.92 (d, 2H), 7.00 (t, 1H), 7.29 (t, 2H).

### EXAMPLE 2A

### Ethyl 2-methylphenoxyacetate

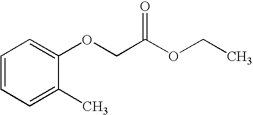

10.81 g (0.10 mol) of 2-methylphenol and 13.82 g (0.10 mol) of potassium carbonate are suspended in 100 ml of N,N-dimethylformamide and stirred at 50° C. for 1 hour. Subsequently, 18.37 g (0.11 mol) of ethyl bromoacetate are added dropwise and the mixture is stirred at 50° C. overnight. After cooling to room temperature, the mixture is concentrated under reduced pressure, taken up with ethyl acetate and washed three times with water. The organic phase is dried over sodium sulfate and freed of solvent under reduced pressure. Distillation of the residue in a Kugelrohr affords 18.5 g (95% of theory) of the desired product.

GC-MS (method 5): Rₜ=12.50 min.

MS (ESIpos): 194 (M)⁺

¹H NMR (300 MHz, CDCl₃): δ=1.29 (t, 3H), 2.29 (s, 3H), 4.26 (q, 2H), 4.62 (s, 2H), 6.70 (d, 1H), 6.89 (dt, 1H), 7.22 (t, 1H), 7.25 (d, 1H).

### EXAMPLE 3A

### Ethyl 2,4-dimethylphenoxyacetate

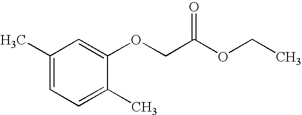

10.00 g (81.86 mmol) of 2,5-dimethylphenol and 11.31 g (81.86 mmol) of potassium carbonate are suspended in 100 ml of N,N-dimethylformamide and stirred at 50° C. for 1 hour. Subsequently, 15.04 g (90.04 mmol) of ethyl bromoacetate are added dropwise and the mixture is stirred at 50° C. overnight. After cooling to room temperature, the mixture is concentrated under reduced pressure, taken up with ethyl acetate and washed three times with water. The organic phase is dried over sodium sulfate and freed of solvent under reduced pressure. 16.96 g (89% of theory) of the desired product is obtained in 89% purity (HPLC).

HPLC (method 1): Rₜ=4.75 min.

MS (DCI): 226 (M+NH₄)⁺

¹H NMR (300 MHz, CDCl₃): δ=1.29 (t, 3H), 2.24 (s, 3H), 2.29 (s, 3H), 4.27 (q, 2H), 4.61 (s, 2H), 6.52 (s, 1H), 6.71 (d, 1H), 7.02 (d, 1H).

### EXAMPLE 4A

### Ethyl 2,3-dimethylphenoxyacetate

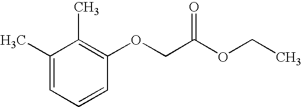

10.00 g (81.86 mmol) of 2,3-dimethylphenol and 16.97 g (122.78 mmol) of potassium carbonate are suspended in 100 ml of N,N-dimethylformamide and stirred at 50° C. for 1 hour. Subsequently, 20.51 g (122.78 mmol) of ethyl bromoacetate are added dropwise and the mixture is stirred at 50° C. overnight. After cooling to room temperature, the mixture is concentrated under reduced pressure, taken up with ethyl acetate and washed three times with water. The organic phase is dried over sodium sulfate and freed of solvent under reduced pressure. 14 g (82% of theory) of the desired product is obtained.

GC (method 6): Rₜ=12.30 min.

MS (DCI): 226 (M+NH₄)⁺

¹H NMR (200 MHz, CDCl₃): δ=1.29 (t, 3H), 2.22 (s, 3H), 2.28 (s, 3H), 4.27 (q, 2H), 4.61 (s, 2H), 6.58 (d, 1H), 6.81 (d, 1H), 7.03 (t, 1H).

### EXAMPLE 5A

### Ethyl 3-methylphenoxyacetate

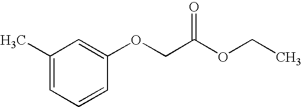

10.00 g (92.47 mmol) of m-cresol and 19.17 g (138.20 mmol) of potassium carbonate are suspended in 100 ml of N,N-dimethylformamide and stirred at 50° C. for 1 hour. Subsequently, 23.16 g (138.71 mmol) of ethyl bromoacetate are added dropwise and the mixture is stirred at 50° C. overnight. After cooling to room temperature, the mixture is concentrated under reduced pressure, taken up with ethyl acetate and washed three times with water. The organic phase is dried over sodium sulfate and freed of solvent under reduced pressure. 16.8 g (94% of theory) of the desired product is obtained.

GC (method 6): Rₜ=11.05 min.

MS (DCI): 212 (M+NH₄)⁺

¹H NMR (200 MHz, CDCl₃): δ=1.29 (t, 3H), 2.32 (s, 3H), 4.27 (q, 2H), 4.60 (s, 2H), 6.70 (d, 1H), 6.72 (s, 1H), 6.80 (d, 1H), 7.18 (t, 1H).

### EXAMPLE 6A

### Ethyl 4-chlorosulfonylphenoxyacetate

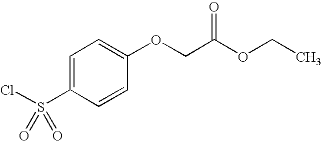

145.49 g (1.25 mol) of chlorosulfonic acid are added dropwise at 0° C. to a solution of 45.00 g (0.25 mol) of ethyl phenoxyacetate in 100 ml of chloroform. The reaction mixture is stirred after the addition at room temperature overnight, then added to ice, and the aqueous phase is extracted three times with methylene chloride. The combined organic phases are washed three times with water, once with saturated sodium hydrogencarbonate solution and once more with water, dried over sodium sulfate and freed of solvent under reduced pressure. 50.7 g (73% of theory) of the desired compound are obtained as a crystalline solid.

GC (method 6): Rₜ=11.38 min.

MS (ESIpos): 278 (M)⁺

¹H NMR (400 MHz, CDCl₃): δ=1.32 (t, 3H), 4.29 (q, 2H), 4.73 (s, 2H), 7.05 (d, 2H), 7.99 (d, 2H).

### EXAMPLE 7A

### Ethyl 4-chlorosulfonyl-2-methylphenoxyacetate

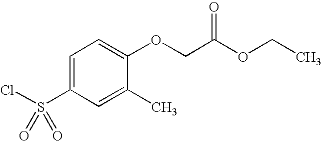

102.59 g (880.40 mmol) of chlorosulfonic acid are added dropwise at 0° C. to a solution of 17.10 g (88.04 mmol) of ethyl 2-methylphenoxyacetate in 100 ml of chloroform. The reaction mixture is stirred after the addition at room temperature overnight, then added to ice, and the aqueous phase is extracted three times with methylene chloride. The combined organic phases are washed three times with water, once with saturated sodium hydrogencarbonate solution and once more with water, dried over sodium sulfate and freed of solvent under reduced pressure. 24.1 g (94% of theory) of the desired compound are obtained as a crystalline solid.

GC (method 6): Rₜ=17.53 min.

MS (DCI): 310 (M+NH₄)⁺

¹H NMR (400 MHz, CDCl₃): δ=1.32 (t, 3H), 2.38 (s, 3H), 4.29 (q, 2H), 4.76 (s, 2H), 6.80 (d, 1H), 7.83 (s, 1H), 7.85 (d, 1H).

### EXAMPLE 8A

### Ethyl 4-chlorosulfonyl-2,5-dimethylphenoxyacetate

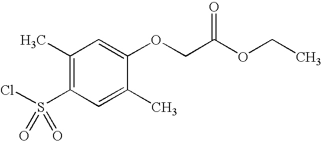

84.44 g (724.67 mmol) of chlorosulfonic acid are added dropwise at 0° C. to a solution of 16.90 g (72.47 mmol) of ethyl 2,5-dimethylphenoxyacetate in 50 ml of chloroform. The reaction mixture is stirred after the addition at room temperature overnight, then added to ice, and the aqueous phase is extracted three times with methylene chloride. The combined organic phases are washed three times with water, once with saturated sodium hydrogencarbonate solution and once more with water, dried over sodium sulfate and freed of solvent under reduced pressure. 23 g (92% of theory) of the desired compound are obtained as an oil.

MS (DCI): 324 (M+NH₄)⁺

¹H NMR (400 MHz, DMSO-d₆): δ=1.20 (t, 3H), 2.12 (s, 3H), 2.42 (s, 3H), 4.16 (q, 2H), 4.78 (s, 2H), 6.60 (s, 1H), 7.48 (s, 1H).

### EXAMPLE 9A

### Ethyl 4-chlorosulfonyl-2,3-dimethylphenoxyacetate

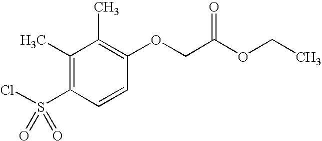

39.17 g (336.13 mmol) of chlorosulfonic acid are added dropwise at 0° C. to a solution of 14.00 g (67.23 mmol) of ethyl 2,3-dimethylphenoxyacetate in 150 ml of chloroform. The reaction mixture is stirred after the addition at room temperature overnight, then added to ice, and the aqueous phase is extracted three times with methylene chloride. The combined organic phases are washed three times with water, once with saturated sodium hydrogencarbonate solution and once more with water, dried over sodium sulfate and freed of solvent under reduced pressure. 19.3 g (94% of theory) of the desired compound are obtained as an oil.

GC-MS (method 4): Rₜ=12.55 min.

MS (DCI): 324 (M+NH₄)⁺

¹H NMR (300 MHz, CDCl₃): δ=1.30 (t, 3H), 2.30 (s, 3H), 2.71 (s, 3H), 4.28 (q, 2H), 4.73 (s, 2H), 6.65 (d, 1H), 7.94 (d, 1H).

### EXAMPLE 10A

### Ethyl 4-chlorosulfonyl-3-methylphenoxyacetate

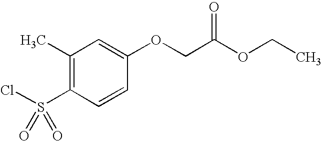

50.39 g (432.48 mmol) of chlorosulfonic acid are added dropwise at 0° C. to a solution of 16.80 g (86.50 mmol) of ethyl 3-methylphenoxyacetate in 50 ml of chloroform. The reaction mixture is stirred after the addition at room temperature overnight, then added to ice, and the aqueous, phase is extracted three times with methylene chloride. The combined organic phases are washed three times with water, once with saturated sodium hydrogencarbonate solution and once more with water, dried over sodium sulfate and freed of solvent under reduced pressure. 9 g (36% of theory) of the desired compound are obtained as an oil.

GC-MS (method 4): Rₜ=11.78 min.

MS (DCI): 310 (M+NH₄)⁺

¹H NMR (300 MHz, CDCl₃): δ=1.31 (t, 3H), 2.74 (s, 3H), 4.28 (q, 2H), 4.70 (s, 2H), 6.80 (d, 1H), 6.90 (d, 1H), 8.02 (d, 1H).

### EXAMPLE 11A

### 4-Benzyloxybenzonitrile

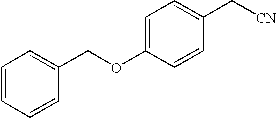

A suspension of 40.17 g (0.29 mol) of potassium carbonate and 38.70 g (0.29 mol) of 4-hydroxybenzonitrile in 200 ml of acetone is heated to reflux for 1 hour. After cooling to room temperature, 52.20 g (0.31 mol), of benzyl bromide in 100 ml of acetone are added and the reaction mixture is heated to reflux overnight. After cooling to room temperature, the mixture is added to water with vigorous stirring, and the precipitate which forms is filtered off, washed with water and petroleum ether and dried at 60° C. under reduced pressure. 61.9 g (95% of theory) of the desired product are obtained as a colorless solid.

GC: Rₜ=4.53 min.

¹H NMR (300 MHz, DMSO-d₆): δ=3.92 (s, 2H), 5.11 (s, 2H), 7.02 (dd, 2H), 7.27 (d, 2H), 7.30-7.48 (m, 5H).

### EXAMPLE 12A

### 1-(4′-Bromophenyl)cyclopentylnitrile

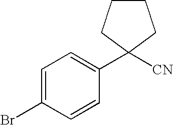

A suspension of 14.60 g (260.14 mmol) of potassium hydroxide in 100 ml of dimethyl sulfoxide is added with stirring and ice cooling to a solution of 15.00 g (76.51 mmol) of 4-bromophenylacetonitrile and 17.35 g (80.34 mmol) of 1,4-dibromobutane in 100 ml of diethyl ether. On completion of addition, the mixture is stirred at room temperature for 6 h, then admixed with water and ethyl acetate while cooling, the phases are separated and the aqueous phase is extracted with ethyl acetate. The combined organic phases are dried over sodium sulfate and freed of solvent under reduced pressure. 19 g (99% of theory) of the desired product are obtained as a colorless oil.

HPLC (method 1): Rₜ=5.01 min.

MS (DCI): 267 (M+NH₄)⁺

¹H NMR (300 MHz, CDCl₃): δ=1.89-2.09 (m, 6H), 2.48 (m, 2H), 7.32 (dt, 2H), 7.51 (dt, 2H).

### EXAMPLE 13A

### 1-(4-Bromophenyl)cyclohexylnitrile

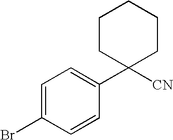

Analogously to the preparation of 1-(4-bromophenyl)cyclopentylnitrile, 15.00 g (76.51 mmol) of 4-bromophenylacetonitrile, 19.04 g (80.34 mmol) of 1,5-dibromopentane and 14.60 g (260.14 mmol) of potassium hydroxide in 100 ml of diethyl ether and 100 ml of dimethyl sulfoxide are used to obtain 20 g (99% of theory) of the desired product as a colorless oil.

HPLC (method 1): Rₜ=5.10 min.

MS (ESIpos): 263 (M)⁺

¹H NMR (300 MHz, CDCl₃) δ=1.67-1.93 (m, 8H), 2.13 (dm, 2H), 7.35 (dt, 2H), 7.51 (dt, 2H).

### EXAMPLE 14A

### 2-(4-Benzyloxyphenyl)-2-methylpropionitrile

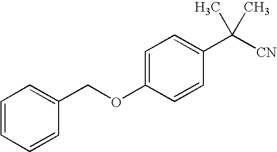

1.00 g (4.48 mmol) of [4-(benzyl xy)phenyl]acetonitrile and 1.33 g (9.411 mmol) of iodomethane are dissolved in 10 ml of diethyl ether and cooled to 0° C. Subsequently, 0.85 g (15.23 mmol) of potassium hydroxide are suspended in 6.36 ml of dimethyl sulfoxide and added and the mixture is stirred at room temperature overnight. The mixture is added to ice and the aqueous phase is extracted with ethyl acetate. The organic phase is dried over sodium sulfate and freed of solvent under reduced pressure. The resulting crystals are recrystallized with ethanol. 1.01 g (90% of theory) of the desired product are obtained in 83% purity.

HPLC (method 1): Rₜ=5.07 min.

MS (DCI): 269 (M+NH₄)⁺

¹H NMR (300 MHz, CDCl₃): δ=1.70 (s, 6H), 5.07 (s, 2H), 6.97 (dt, 2H), 7.31-7.45 (m, 7H).

### EXAMPLE 15A

### 1-(4-B enzyloxyphenyl)cyclobutylnitrile

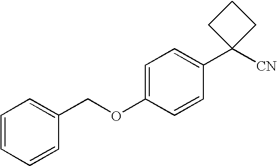

11.16 g (50.00 mmol) of [4-(benzyloxy)phenyl]acetonitrile and 10.60 g (52.50 mmol) of 1,3-dibromopropane are dissolved in 100 ml of diethyl ether and cooled to 0° C. Subsequently, 9.54 g (170 mmol) of potassium hydroxide are suspended in 71 ml of dimethyl sulfoxide and added and the mixture is stirred at room temperature overnight. The mixture is added to ice and the aqueous phase is extracted with ethyl acetate. The organic phase is dried over sodium sulfate and freed of solvent under reduced pressure. The residue is purified using silica gel 60 (eluent: methylene chloride and 100:5 methylene chloride/methanol). The clean fractions are combined and freed of solvent under reduced pressure. 6.9 g (52% of theory) of the desired product are obtained.

LC-MS (method 3): Rₜ=2.93 min.

MS (ESIpos): m/z=264 (M+H)⁺

¹H NMR (200 MHz, CDCl₃): δ=1.94-2.15 (m, 2H), 2.49-2.67 (m, 2H), 2.73-2.89 (m, 2H), 5.07 (s, 2H), 6.99 (d, 2H), 7.29-7.46 (m, 7H).

### EXAMPLE 16A

### 1-(4-Benzyloxyphenyl)cyclopentylnitrile

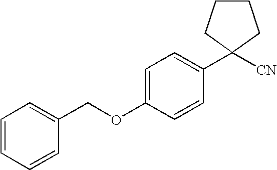

11.16 g (50.00 mmol) of [4-(benzyloxy)phenyl]acetonitrile and 11.34 g (52.50 mmol) of 1,4-dibromobutane are dissolved in 100 ml of diethyl ether and cooled to 0° C. Subsequently, 9.54 g (170 mmol) of potassium hydroxide are suspended in 71 ml of dimethyl sulfoxide and added and the mixture is stirred at room temperature overnight. The mixture is added to ice and the aqueous phase is extracted with ethyl acetate. The organic phase is dried over sodium sulfate and freed of solvent under reduced pressure. The residue is recrystallized from ethanol. 11.6 g (84% of theory) of the desired product are obtained.

HPLC (Method 1): Rₜ=5.22 min

MS (ESIpos): m/z=278 (M+H)⁺

¹H NMR (300 MHz, CDCl₃): δ=1.85-2.10 (m, 6H), 2.39-2.51 (m, 2H), 5.07 (s, 2H), 6.97 (d, 2H), 7.31-7.45 (m, 7H).

### EXAMPLE 17A

### 1-(4-Benzyloxyphenyl)cyclohexylnitnile

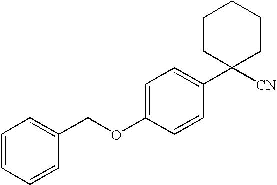

11.16 g (50.00 mmol) of [4-(benzyloxy)phenyl]acetonitrile and 12.07 g (52.50 mmol) of 1,4-dibromopentane are dissolved in 100 ml of diethyl ether and cooled to 0° C. Subsequently, 9.54 g (170 mmol) of potassium hydroxide are suspended in 71 ml of dimethyl sulfoxide and added and the mixture is stirred at room temperature overnight. The mixture is added to ice and crystals which form are filtered off with suction, washed with water and ethanol and dried (product fraction 1). The aqueous phase is extracted with ethyl acetate. The organic phase is dried over sodium sulfate and freed of solvent under reduced pressure. The residue is purified using silica gel 60 (eluent: methylene chloride and 100:5 methylene chloride methanol). The clean fractions are combined and freed of solvent under reduced pressure (product fraction 2). A total of 10.56 g (72% of theory) of the desired product are obtained.

LC-MS (method 3): Rₜ=3.10 min.

MS (DCI): m/z=309 (M+NH₄)⁺

¹H NMR (200 MHz, DMSO-d₆): δ=1.12-1.42 (m, 2H), 1.45-1.92 (m, 6H), 2.04 (d, 2H), 5.11 (s, 2H), 7.05 (dd, 2H), 7.19-7.55 (m, 7H).

### EXAMPLE 18A

### 2-(4-Methoxyphenyl)-4-phenylbutanenitrile

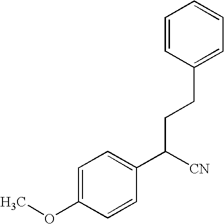

20.00 g (135.89 mmol) of 4-methoxyphenylacetonitrile are dissolved in 12 ml of diethyl ether and admixed with 26.41 g (19.49 ml; 142.69 mmol) of 2-phenylethyl bromide. The mixture is cooled to 0° C., then 25.92 g; (462 mmol) of potassium hydroxide are suspended in 80 ml of dimethyl sulfoxide with ice cooling and added. The mixture is stirred at room temperature overnight. For workup, the mixture is cooled once again and diluted with toluene and water. The phases are separated and the aqueous phase is washed twice more with toluene. The combined organic phases are dried over sodium sulfate, filtered and freed of solvent under reduced pressure. The residue is purified using 400 g of silica gel 60 (eluent: cyclohexane and 98:2 cyclohexane/ethyl acetate). The clean fractions are combined and freed of solvent under reduced pressure. 15.6 g (46% of theory) of the desired product are obtained in 86% purity.

HPLC (method 1): Rₜ=4.96 min.

MS (DCI): m/z=269 (M+NH₄)⁺

¹H NMR (300 MHz, CDCl₃): δ=2.05-2.31 (m, 2H), 2.74-2.85 (m, 2H), 3.64-3.72 (m, 1H), 3.80 (s, 3H), 6.89 (d, 2H), 7.15-7.34 (m, 7H).

### EXAMPLE 19A

### [1-(4-Bromophenyl)cyclopentyl]methylamine

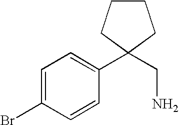

A solution of 6.27 g (287.84 mmol) of lithium borohydride in 50 ml of THF is admixed slowly with stirring at room temperature with 46.91 g (431.76 mmol) of chlorotrimethylsilane. Subsequently, a solution of 18.00 g (71.96 mmol) of 1-(4-bromophenyl)cyclopentylnitrile in 10 ml of THF is added with ice cooling, the mixture is heated to reflux for 4 h and then stirred at room temperature overnight. The mixture is then admixed with methanol and made alkaline with 2 N sodium hydroxide solution, and the aqueous phase is extracted twice with methylene chloride. The combined organic extracts are dried over sodium sulfate and freed of solvent under reduced pressure, and the resulting residue is purified chromatographically on silica gel (10:1 cyclohexane/ethyl acetate→100:5 methylene chloride/methanol+ammonia solution). 5.1 g (23% of theory) of the desired product are obtained in 82% purity as an oil.

HPLC (method 1): Rₜ=3.91 min.

MS (DCI): 254 (M+H)⁺

¹H NMR (200 MHz, CDCl₃): δ=1.50 (broad, s, 2H), 1.71 (m, 4H), 1.90 (m, 4H), 2.72 (s, 2H), 7.16 (dt, 2H), 7.42 (dt, 2H).

### EXAMPLE 20A

### [1-(4-Bromophenyl)cyclohexyl]methylamine

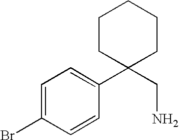

Analogously to the preparation of [1-(4-bromophenyl)cyclopentyl]methylamine, 4.95 g (227.13 mmol) of lithium borohydride, 37.01 g (340.70 mmol) of chlorotrimethylsilane and 20.00 g (75.71 mmol) of 1-(4-bromophenyl)cyclohexylnitrile are used to obtain 15.28 g (56% of theory) of the desired product in 74% purity (HPLC) as an oil.

HPLC (method 1): Rₜ=4.11 min.

MS (DCI): 254 (M+H)⁺

¹H NMR (400 MHz, CDCl₃): δ=1.18 (broad, s, 2H), 1.37 (m, 4H), 1.54 (m, 4H), 2.09 (dd, 2H), 2.68 (s, H), 7.21 (dt, 2H), 7.45 (dt, 2H).

### EXAMPLE 21A

### 2-[4-(Benzyloxy)phenyl]-2-methylpropylamine

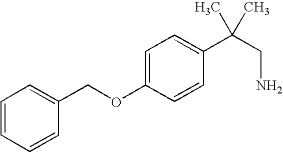

A solution of 28.60 g (113.80 mmol) of 2-[4-(benzyloxy)phenyl]-2-methyl-propanenitrile in 550 ml of tetrahydrofuran is admixed slowly with 114.00 ml of a 1 molar lithium aluminum hydride solution in tetrahydrofuran. The mixture is boiled under reflux for 15 minutes. The reaction mixture is then cooled to 0° C. and admixed with 20% potassium sodium tartrate solution. The mixture is diluted with water and extracted with ethyl acetate. The organic phase is washed with saturated sodium chloride solution, dried over sodium sulfate and freed of solvent under reduced pressure. The resulting crystalline solid is recrystallized from ethanol. 27.8 g (96% of theory) of the desired product is obtained with 99% purity.

HPLC (method 1): Rₜ=4.18 min.

MS (DCI): m/z 273 (M+NH₄)⁺

¹H NMR (400 MHz, CDCl₃): δ=0.80-1.1 (m, 2H), 1.28 (s, 6H), 2.76 (s, 2H), 5.05 (s, 2H), 6.94 (d, 2H), 7.23-7.28 (m, 2H), 7.29-7.46 (m, 5H).

### EXAMPLE 22A

### {1-[4-(Benzyloxy)phenyl]cyclobutyl}methylamine

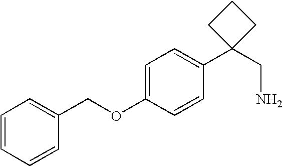

Under argon supply, 26.20 ml (26.20 mmol) of a 1 molar lithium aluminum hydride solution in tetrahydrofuran are added dropwise at room temperature to a solution of 6.90 g (26.20=mol) of 1-[4-(benzyloxy)phenyl]cyclobutanecarbonitrile in 200 ml of tetrahydrofuran. The mixture is boiled under reflux overnight. The reaction mixture is cooled to 0° C. and admixed with 20% potassium sodium tartrate solution. The resulting solid is filtered off with suction and washed with water and ethyl acetate and discarded. The two phases present in the filtrate are separated. The aqueous phase is extracted three times more with ethyl acetate. The combined organic phases are dried over sodium sulfate and freed of solvent under reduced pressure. 6.60 g (94% of theory) of the desired product are obtained with 93% purity.

HPLC (method 1): Rₜ=4.24 min.

MS (DCI): m/z 285 (M+NH₄)⁺

¹H NMR (200 MHz, DMSO-d₆): δ=0.90-1.15 (m, 2H), 1.63-2.02 (m, 2H), 2.70 (s, 2H), 5.06 (s, 2H), 6.97 (q, 4H), 7.27-7.50 (m, 5H).

### EXAMPLE 23A

### {1-[4-(Benzyloxy)phenyl]cyclopentyl}methylamine

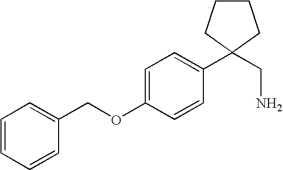

Under argon supply, 36.05 ml (36.05 mmol) of a 1 molar lithium aluminum hydride solution in tetrahydrofuran are added dropwise at room temperature to a solution of 10.00 g (36.05 mmol) of 1-[4-(benzyloxy)phenyl]cyclopropylcarbonitrile in 100 ml of tetrahydrofuran. The mixture is boiled under reflux overnight. The reaction mixture is cooled to 0° C. and admixed with 20% potassium sodium tartrate solution. The resulting solid is filtered off with suction and washed with water and ethyl acetate and discarded. The two phases present in the mother liquor are separated. The aqueous phase is extracted three times more with ethyl acetate. The combined organic phases are dried over sodium sulfate and freed of solvent under reduced pressure. The mixture is purified using silica gel 60 (eluent: 100:2 methylene chloride/methanol). The clean fractions are combined and freed of solvent under reduced pressure. 9.70 g (96% of theory) of the desired product are obtained with 98% purity.

HPLC (method 1): Rₜ=4.32 min.

MS (ESIpos): m/z=282 (M+H)⁺

¹H NMR (200 MHz, CDCl₃): δ=1.07-1.20 (m, 2H), 1.61-1.75 (m, 4H), 1.80-1.93 (m, 4H), 2.70 (s, 2H), 5.05 (s, 2H), 6.93 (d, 2H), 7.19 (d, 2H), 7.29-7.49 (m, 5H).

### EXAMPLE 24A

### {1-[4-(Benzyloxy)phenyl]cyclohexyl}methylamine

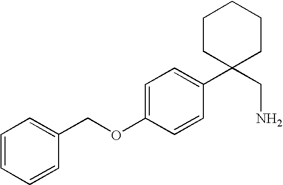

Under argon supply, 29.17 ml (29.17 mmol) of a 1 molar lithium aluminum hydride solution in tetrahydrofuran are added dropwise at room temperature to a solution of 8.50 g (29.17 mmol) of 1-[4-(benzyloxy)phenyl]cyclohexanecarbonitrile in 90 ml of tetrahydrofuran. The mixture is boiled under reflux overnight. The reaction mixture is cooled to 0° C. and admixed with 20% potassium sodium tartrate solution. The resulting solid is filtered off with suction and washed with water and ethyl acetate and discarded. The two phases present in the filtrate are separated. The aqueous phase is extracted three times more with ethyl acetate. The combined organic phases are dried over sodium sulfate and freed of solvent under reduced pressure. The mixture is purified using silica gel 60 (eluent: 100:2 methylene chloride/methanol). The clean fractions are combined and freed of solvent under reduced pressure. 7.20 g (84% of theory) of the desired product are obtained with 95% purity.

HPLC (method 1): Rₜ=4.44 min.

MS (ESIpos): m/z=296 (M+H)⁺

¹H NMR (200 MHz, CDCl₃): δ=1.07-1.21 (m, 2H), 1.64-1.74 (m, 4H), 1.80-1.93 (m, 4H), 2.70 (s, 2H), 5.05 (s, 2H), 6.95 (d, 2H), 7.19 (d, 2H); 7.30-7.49 (m, 5H).

### EXAMPLE 25A

### 2-(4-Methoxyphenyl)-4-phenylbutylamine

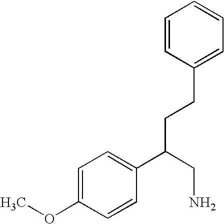

Under argon supply, 31.33 ml (31.33 mmol) of a 1 molar lithium aluminum hydride solution in tetrahydrofuran were added dropwise at 0° C. to a solution of 7.50 g (29.84 mmol) of 2-(4-methoxyphenyl)-4-phenylbutanenitrile in 150 ml of tetrahydrofuran. The mixture is allowed to come slowly to room temperature and it is stirred further for 24 hours. The reaction mixture is cooled to 0° C., admixed with 20% potassium sodium tartrate solution and washed with water and ethyl acetate. The phases are separated. The ethyl acetate phase is extracted once more with saturated sodium chloride solution. The organic phase is dried over sodium sulfate and freed of solvent under reduced pressure. Purification is effected through 200 g of silica gel 60 (eluent: cyclohexane/ethyl acetate and later with 9:1 methylene chloride/ethanol). The clean fractions are combined and freed of solvent under reduced pressure. 3.35 g (44% of theory) of the desired product are obtained with 96% purity.

HPLC (method 1): Rₜ=4.15 min.

MS (ESIpos): m/z=256 (M+H)⁺

¹H NMR (200 MHz, CDCl₃): δ=1.13-1.44 (m, 2H), 1.77-2.04 (m, 2H), 2.42-2.67 (m, 3H), 2.74-3.00 (m, 2H), 3.81 (s, 3H), 6.89 (d, 2H), 7.05-7.32 (m, 7H).

### EXAMPLE 26A

### 2-(4-Methoxyphenyl)ethylacetamide

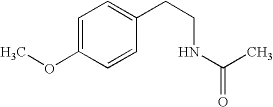

25.00 g (165.34 mmol) of 2-(4-methoxyphenyl)ethylamine and 70.00 ml of acetic acid are initially charged in 1 l of xylene. The mixture is boiled on a water separator for 2 hours. The mixture is freed of solvent under reduced pressure. 31.00 g (97% of theory) of the desired product are obtained.

HPLC (method 1): Rₜ=3.51 min.

MS (DCI): m/z=211 (M+NH₄)⁺

¹H NMR (300 MHz, CDCl₃): δ=1.93 (s, 3H), 2.75 (t, 2H), 3.47 (q, 2H), 3.79 (s, 3H), 5.48 (s, 1H), 6.84 (d, 2H), 7.10 (d, 2H).

### EXAMPLE 27A

### [1-(4-Bromophenyl)cyclopentyl]methylformamide

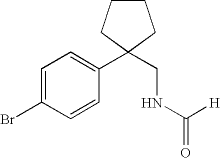

A solution of 5.10 g (82% pure, 16.45 mmol) of [1-(4-bromophenyl)-cyclopentyl]-methylamine and 1.52 g (32.91 mmol) of formic acid in 50 ml of xylene is heated on a water separator under reflux for 6 h and subsequently stirred at room temperature overnight. The mixture is concentrated under reduced pressure, the residue is taken up with ethyl acetate, and the organic phase is washed with sodium hydrogencarbonate solution and dried over sodium sulfate. After the solvent has been removed under reduced pressure, 4.9 g (99% of theory) of the desired product are obtained as a yellow solid.

HPLC (method 1): Rₜ 4.52 min.

MS (DCI): 299 (M+NH₄)⁺

¹H NMR (300 MHz, CDCl₃): δ=1.68-2.00 (m, 8H), 3.22+3.44 (d, 2H), 5.10+5.30 (broad, s, 1H), 7.11+7.18 (dt, 2H), 7.46 (dt, 2H), 8.10 (s, 1H).

### EXAMPLE 28A

### [1-(4-Bromophenyl)cyclohexyl]methylformamide

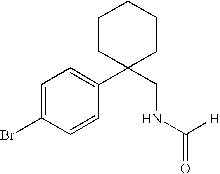

A solution of 15.28 g (71% pure, 40.59 mmol) of [1-(4-bromophenyl)cyclohexyl]-methylamine in 3.74 g (81.18 mmol) of formic acid is admixed with 3 ml of xylene and molecular sieve, heated to reflux for 6 h and subsequently stirred at room temperature overnight. The mixture is admixed with ethyl acetate, and the organic phase is washed with sodium hydrogencarbonate solution, water and saturated sodium chloride solution and dried over sodium sulfate. After the solvent has been removed under reduced pressure, the residue is purified using silica gel (1:1 cyclohexane/ethyl acetate). 4.5 g (77% pure by HPLC, 29% of theory) of the desired product are obtained as a colorless solid.

HPLC (method 1): Rₜ=4.62 min.

MS (ESIpos): 296 (M+H)⁺

¹H NMR (200 MHz, CDCl₃): δ=1.25-1.80 (m, 8H), 2.10 (m, 2H), 3.19+3.40 (d, 2H), 5.02+5.23 (broad, s, 1H), 7.18+7.23 (d, 2H), 7.50 (d, 2H), 8.10 (s, 1H).

### EXAMPLE 29A

### 2-[4-(Benzyloxy)phenyl]-2-methylpropylformamide

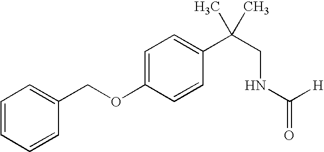

27.60 g (0.108 mol) of 2-[4-(benzyloxy)phenyl]-2-methylpropylamine and 124.36 g (2.702 mol) of formic acid are initially charged in 300 ml of xylene. The mixture is boiled on a water separator for 3 hours. The mixture is concentrated under reduced pressure and the residue dissolved in ethyl acetate and extracted with water. The organic phase is washed twice more with water, dried over sodium sulfate and freed of solvent under reduced pressure. The mixture is filtered through, silica gel 60. The clean fractions are combined and freed of solvent under reduced pressure. 24.21 g (79% of theory) of the desired product are obtained in 98% purity.

HPLC (method 1): Rₜ=4.12 min.

MS (DCI): m/z=301 (M+NH)⁺

¹H NMR (200 MHz, CDCl₃): δ=1.32 (s, 6H), 3.48 (d, 2H), 5.05 (s, 0.2H), 6.90-7.01 (dt, 2H), 7.18-7.49 (m, 7H), 7.86 (d, 1H), 8.10 (s, 1H).

### EXAMPLE 30A

### {1-[4-(Benzyloxy)phenyl]cyclobenzyl}methylformamide

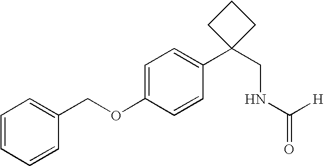

6.60 g (24.68 mmol) of {1-[4-benzyloxy)phenyl]cyclobutyl}methylamine and 11.36 g (246.85 mmol) of formic acid are initially charged in 100 ml of xylene. The mixture is boiled on a water separator. The mixture is concentrated under reduced pressure and the residue dissolved in ethyl acetate and extracted with water. The organic phase is washed twice more with water, dried over sodium sulfate and freed of solvent under reduced pressure. 7.20 g (99% of theory) of the desired product are obtained in 93% purity.

HPLC (method 1): Rₜ 4.64 min.

MS (DCI): m/z=313 (M+NH₄)⁺

¹HNMR (300 MHz, CDCl₃): δ=1.80-1.99 (m, 2H) 2.02-2.21 (m, 2H), 3.63 (d, 2H), 5.05 (s, 2H), 6.89-7.01 (m, 2H), 7.29-7.46 (m, 5H), 7.85 (d, 1H), 8.13 (s, 1H).

### EXAMPLE 31A

### {1-[4-(Benzyloxy)phenyl]cyclopentyl}methylformamide

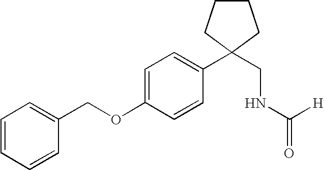

18.40 g (65.39 mmol) of {1-[4-benzyloxy)phenyl]cyclopentyl}methylamine and 75.24 g (1634.70 mmol) of formic acid are initially charged in 200 ml of xylene. The mixture is boiled on a water separator. The mixture is concentrated under reduced pressure and the residue dissolved in ethyl acetate and extracted with water. The organic phase is washed twice more with water, dried over sodium sulfate and freed of solvent under reduced pressure. 20.00 g (99% of theory) of the desired product are obtained in 100% purity.

HPLC (method 1): Rₜ=4.75 min.

MS (ESIpos): m/z=310 (M+H)⁺

¹H NMR (300 MHz, CDCl₃): δ=1.64-1.95 (m, 10H), 3.42 (d, 2H), 5.05 (s, 2H), 6.90-6.99 (m, 2H), 7.10-7.22 (m, 2H), 7.29-7.48 (m, 5H), 7.78 (d, 1H), 8.09 (s, 1H).

### EXAMPLE 32A

### {1-[4-(Benzyloxy)phenyl]cyclohexyl}methylformamide

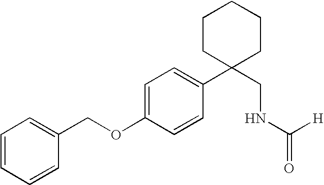

9.20 g (31.14 mmol) of {1-[4-benzyloxy)phenyl]cyclohexyl}methylamine and 14.33 g (311.42 mmol) of formic acid are initially charged in 100 ml of xylene. The mixture is boiled on a water separator. The mixture is concentrated under reduced pressure and the residue dissolved in ethyl acetate and extracted with water. The organic phase is washed twice more with water, dried over sodium sulfate and freed of solvent under reduced pressure. 10.62 g (90% of theory) of the desired product are obtained in 85% purity.

HPLC (method 1): Rₜ=4.83 min.

MS (DCI): m/z 341 (M+NH₄)⁺

¹H NMR (300 MHz, CDCl₃): δ=1.29-1.70 (m, 8H), 1.95-2.15 (m, 2H), 3.38 (dd, 2H), 5.05 (s, 2H), 6.89-6.97 (m, 2H), 7.11-7.23 (m, 2H), 7.28-7.47 (m, 5H), 7.78 (d, 1H), 8.08 (s, 1H).

### EXAMPLE 33A

### 2-(4-Methoxyphenyl)-4-phenylbutylformamide

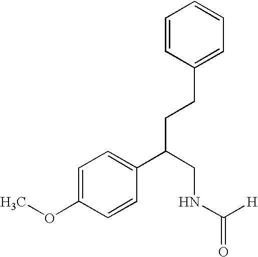

3.49 g (13.67 mmol) of 2-(4-methoxyphenyl)-4-phenylbutylamine and 10 ml of formic acid are initially charged in 200 ml of xylene. The mixture is boiled on the water separator for 3 hours; The mixture is concentrated under reduced pressure and the residue admixed twice more with toluene and in each case freed again of solvent under reduced pressure. 3.68 g (95% of theory) of the desired product are obtained in 93% purity.

HPLC (method 1): Rₜ=4.41 min.

MS (DCI): m/z=301 (M+NH₄)⁺

¹H NMR (300 MHz, CDCl₃): δ=1.79-2.05 (m, 2H), 2.33 (d, 1H), 2.34-2.54 (m, 2H), 2.67-2.80 (m, 1H), 3.15-3.27 (m, 1H), 3.81 (s, 3H), 5.27 (m, 1H), 6.89 (d, 2H), 7.03-7.30 (m, 7H, 8.04 (s, 1H).

### EXAMPLE 34A

### [1-(1,1′-Biphenyl-4-yl)cyclopentyl]methylformamide

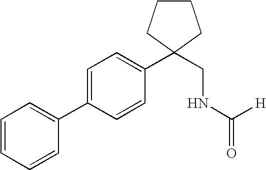

A solution of 50 mg (0.07 mmol) of bis(triphenylphosphine)palladium dichloride, 500 mg (1.77 mmol) of [1-(4-bromophenyl)cyclopentyl]methylformamide and 280 mg (2.30 mmol) of benzeneboronic acid in 10 ml of acetonitrile/DMF (1:1) is heated to 70° C. for 1 h. Subsequently, 2 ml of 2 M sodium carbonate solution are added and the reaction mixture is heated to 100° C. overnight. After cooling, the mixture is, concentrated under reduced pressure and the crude product purified, by preparative HPLC. 120 mg (24% of theory) of the desired product are obtained as a colorless solid.

HPLC (method 1): Rₜ=4.79 min.

MS (DCI): 297 (M+NH₄)⁺

¹H NMR (200 MHz, CDCl₃): δ=1.70-2.08 (m, 8H), 3.29+3.50 (d, 2H), 5.20+5.35 (broad, s, 1H), 7.30-7.50 (m, 5H), 7.58 (m, 4H), 7.83 (d)+8.12 (s, 1H).

### EXAMPLE 35A

### [1-(4′-Fluoro-1,1′-biphenyl-4-yl)cyclopentyl]methylformamide

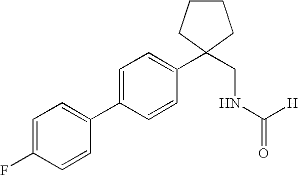

Analogously to the preparation of [1-(1,1′-biphenyl-4-yl)cyclopentyl]methylformamide, 500 mg (1.772 mmol) of [1-(4-bromophenyl)cyclopentyl methylformamide, 50 mg (0.07 mmol) of bis(triphenylphosphine)palladium dichloride and 322 mg (2.304 mmol) of 4-fluorobenzeneboronic acid are used to obtain 229 mg (43% of theory) of the desired product as a white solid.

HPLC (method 1): Rₜ=4.83 min.

MS (ESIpos): 298 (M+H)⁺

¹H NMR (300 MHz, d₆-DMSO): δ=1.55-2.00 (m, 8H), 3.10+3.31 (d, 2H), 7.23-7.40 (m, 4H), 7.58 (d, 2H), 7.61-7.73 (m, 3H), 7.92 (d, 1H).

### EXAMPLE 36A

### [1-(4′-Trifluoromethyl-1,1′-biphenyl-4-yl)cyclopentyl]methylformamide

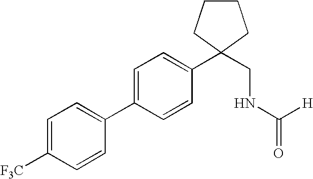

Analogously to the preparation of [1-(1,1′-biphenyl-4-yl)cyclopentyl]methylformamide, 500 mg (1.772 mmol) of [1-(4-bromophenyl)cyclopentyl]methylformamide, 50 mg (0.07 mmol) of bis(triphenylphosphine)palladium dichloride and 440 mg (2.304 mmol) of 4-(trifluoromethyl)benzeneboronic acid are used to obtain 378 mg (61% of theory) of the desired product as a white solid.

HPLC (method 1): Rₜ=5.14 min.

MS (ESIpos): 348 (M+H)⁺

¹H NMR (300 MHz, d₆-DMSO): δ=1.55-2.00 (m, 8H), 3.20+3.33 (d, 2H), 7.38+7.43 (d, 2H), 7.68 (d, 3H), 7.80 (d, 2H), 7.89 (d, 2H), 7.91 (d, 1H).

### EXAMPLE 37A

### [1-(1,1′-Biphenyl-4-yl)cyclohexyl]methylformamide

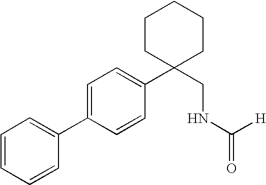

Analogously to the preparation of [1-(1,1′-biphenyl-4-yl)cyclopentyl]methylformamide, 1.00 g (2.60 mmol) of [1-(4-bromophenyl)cyclohexyl]methylformamide, 100 mg (0.14 mmol) of bis(triphenylphosphine)palladium dichloride and 317 mg (2.60 mmol) of benzeneboronic acid are used to obtain 297 mg (36% of theory) of the desired product as a white solid.

HPLC (method 1): Rₜ=4.84 min.

MS (DC): 311 (M+NH₄)⁺

¹H NMR (300 MHz, d₆-DMSO): δ=1.15-1.70 (m, 8H), 2.00-2.17 (m, 2H), 3.09+3.22 (d, 2H), 7.29-7.52 (m, 5H), 7.60-7.72 (m, 4H), 7.92 (d, 1H).

### EXAMPLE 38A

### [1-(4′-Fluoro-1,1′-biphenyl-4-yl)cyclohexyl]methylformamide

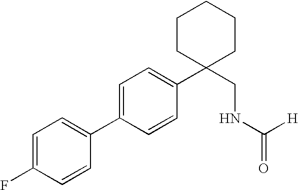

Analogously to the preparation of [1-(1,1′-biphenyl-4-yl)cyclopentyl]methylformamide, 2.086 g (5.42 mmol) of [1-(4-bromophenyl)cyclohexyl]methylformamide, 100 mg (0.14 mmol) of bis(triphenylphosphine)palladium dichloride and 759 mg (5.42 mmol) of 4-fluorobenzeneboronic acid are used to obtain 744 mg (42% of theory) of the desired product as a white solid.

HPLC (method 1): Rₜ=4.87 min.

MS (DCI): 329 (M+NH₄)⁺

¹H NMR (200 MHz, d₆-DMSO): δ=1.15-1.70 (m, 8H), 1.95-2.18 (m, 2H), 3.08+3.22 (d, 2H), 7.20-7.35 (m, 2H), 7.43 (d, 2H), 7.55-7.77 (m, 5H), 7.92 (d, 1H).

### EXAMPLE 39A

### [1-(4′-Trifluoromethyl-1,1′-biphenyl-4-yl)cyclohexyl]methylformamide

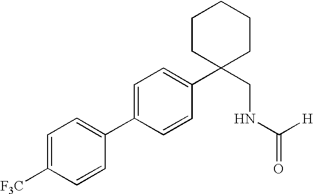

Analogously to the preparation of [1-(1,1′-biphenyl-4-yl)cyclopentyl]methylformamide, 1.00 mg (2.60 mmol) of [1-(4-bromophenyl)cyclohexyl]methylformamide, 100 mg (0.14 mmol) of bis(triphenylphosphine)palladium dichloride and 494 mg (2.60 mmol) of 4-(trifluoromethyl)benzeneboronic acid are used to obtain 225 mg (24% of theory) of the desired product as a white solid.

HPLC (method 1): Rₜ=5.22 min.

MS (DCI): 379 (M+NH₄)⁺

¹H NMR (300 MHz, d₆-DMSO): δ=1.15-1.70 (m, 8H), 2.00-2.20 (m, 2H), 3.10+3.23 (d, 2H), 7.50 (d, 2H), 7.60-7.99 (m, 8H).

### EXAMPLE 40A

### 7-Methoxy-1-methyl-3,4-dihydroisoquinoline

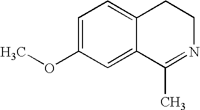

Under argon, 43.0 g (0.222 mol) of 2-(4-methoxyphenyl)ethylacetamide are dissolved in 320 ml of toluene, admixed with 100 ml of phosphorus oxychloride and heated to reflux for 3 hours. After cooling to room temperature, the mixture is admixed with ice-water and ethyl acetate and made alkaline with 20% sodium hydroxide solution, and the aqueous phase is extracted three times with diethyl ether. The combined organic extracts are dried over sodium sulfate and freed of solvent under reduced pressure. 4.1 g (10% of theory) of the desired product are obtained, which are converted without further characterization to 7-methoxy-1-methyl-1,2,3,4-tetrahydroisoquinoline (see Example 53A).

### EXAMPLE 41A

### 7-(Benzyloxy)-1,4,4-trimethyl-3,4-dihydro-2(1H)-isoquinolinecarbaldehyde

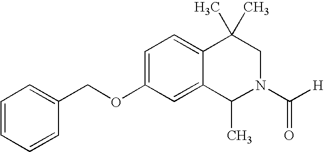

Under argon, 10.00 g (35.29 mmol) of 2-[4-(benzyloxy)phenyl]-2-methylpropylformamide and 39.46 ml (705.79 mmol) of acetaldehyde are initially charged in 300 ml of a mixture of trifluoroacetic acid/acetic acid (2:8) and heated to 100° C. for 4 hours. Afterward, the mixture is cooled to room temperature and the acid mixture is distilled off under reduced pressure. The residue is purified by RP-18 chromatography (acetonitrile/water/0.1% acid, gradient). The clean fractions are combined, concentrated under reduced pressure and dried. 4.41 g (40% of theory) of the desired product are obtained in 100% purity.

HPLC (method 1): Rₜ=4.74 min.

MS (DCI): m/z=327 (M+NH₄)⁺

¹H NMR (300 MHz, CDCl₃): δ=1.15 (s, 3H), 1.35 (s, 3H), 1.46 (d, 3H), 3.17 (d, 1H), 3.38 (d, 1H), 5.04 (s, 2H), 5.42 (q, 1H), 6.63-6.91 (m, 2H), 7.22 (d, 2H), 7.28-7.45 (m, 4H), 8.08 (s, 1H).

### EXAMPLE 42A

### 7-(Benzyloxy)-4-spirocyclobutyl-3,4-dihydro-2(1H)-isoquinolinecarbaldehyde

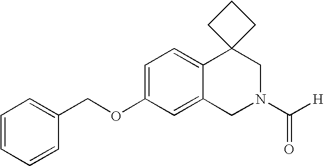

Under argon, 7.20 g (24.38 mmol) of {1-[4-(benzyloxy)phenyl]cyclobutyl}methylformamide and 1.46 g (48.75 mmol) of formaldehyde are initially charged in 20 ml of trifluoroacetic acid and 80 ml of acetic acid. The mixture is boiled under reflux for 3 hours, subsequently cooled, poured onto ice and extracted with methylene chloride. The organic phase is washed twice more with water, once with saturated sodium bicarbonate solution and once with water. Subsequently, the organic phase is dried over sodium sulfate and concentrated under reduced pressure and dried. Purification is effected using silica gel 60 (eluent: methylene chloride and 1:1 cyclohexane/ethyl acetate). The clean fractions are combined and freed of solvent under reduced pressure. 3.10 kg (41% of theory) of the desired product are obtained in 85% purity.

HPLC (method 1): Rₜ=4.84 min.

MS (ESIpos): m/z=308 (M+H)⁺

¹H NMR (200 MHz, CDCl₃):δ=1.83-2.41 (m, 6H), 3.70 (d, 2H), 4.57 (d, 2H), 5.04 (s, 2H), 6.63-6.97 (m, 2H), 7.18-7.58 (m, 6H), 8.27 (d, 1H).

### EXAMPLE 43A

### 7-(Benzyloxy)-4-spirocyclopentyl-3,4-dihydro-2(1H)-isoquinolinecarbaldehyde

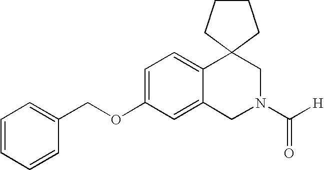

Under argon, 20.00 g (64.64 mmol) of {1-[4-(benzyloxy)phenyl]cyclopentyl}methylformamide and 3.88 g (129.28 mmol) of formaldehyde are initially charged in 35 ml of trifluoroacetic acid and 135 ml of acetic acid. The mixture is boiled under reflux for 30 minutes, subsequently cooled, poured onto ice and extracted with methylene chloride. The organic phase is washed once more with water, twice with saturated sodium bicarbonate solution, twice with water and once with saturated sodium chloride solution. Subsequently, the organic phase is dried over sodium sulfate and concentrated under reduced pressure and dried. Purification is effected using silica gel 60 (eluent: methylene chloride). The clean fractions are combined and freed of solvent under reduced pressure. 20.50 g (99% of theory) of the desired product are obtained.

HPLC (method 1): Rₜ=4.44 min.

MS (ESIpos): m/z 322 (M+H)⁺

¹H NMR (200 MHz, CDCl₃): δ=1.42-2.03 (m, 8H), 3.44 (d, 2H), 4.60 (d, 2H), 5.07 (s, 2H), 6.62-7.00 (m, 2H), 7.33-7.51 (m, 6H), 8.25 (d, 1H).

### EXAMPLE 44A

### 7-(Benzyloxy)-4-spirocyclohexyl-3,4-dihydro-2(1H)-isoquinolinecarbaldehyde

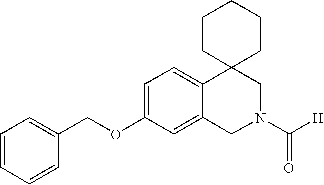

Under argon, 10.00 g (27.82 mmol) of {1-[4-(benzyloxy)phenyl]cyclohexyl}methylformamide and 1.65 g (55.03 mmol) of formaldehyde are initially charged in 20 ml of trifluoroacetic acid and 80 ml of acetic acid. The mixture is boiled under reflux for 30 minutes, subsequently cooled, poured onto ice and extracted with methylene chloride. The organic phase is washed twice more with water, once with saturated sodium bicarbonate solution and once with water. Subsequently, the organic phase is dried over sodium sulfate and concentrated under reduced pressure and dried. Purification is effected using silica gel 60 (eluent: methylene chloride and 1:1 cyclohexane/ethyl acetate). The clean fractions are combined and freed of solvent under reduced pressure. 7.20 g (72% of theory) of the desired product are obtained in 92% purity.

HPLC (method 1): Rₜ=5.05 min.

MS (ESIpos): m/z=336 (M+H)⁺

¹H NMR (200 MHz, CDCl₃): δ=1.10-1.87 (m, 10H), 3.59 (d, 2H), 4.58 (d, 2H), 5.03 (s, 2H), 6.61-7.01 (m, 2H), 7.21-7.48 (m, 6H), 8.24 (d, 1H).

### EXAMPLE 45A

### 7-Phenyl-4-spirocyclopentyl-3,4-dihydro-2(1H)-isoquinolinecarbaldehyde

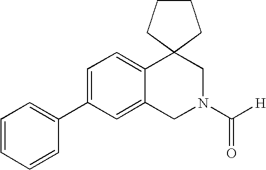

A mixture of 103 mg (0.369 mmol) of [1-(1,1′-biphenyl-4-yl)cyclopentyl]methylformamide and 55 μl (0.737 mmol) of 37% formaldehyde solution is admixed with 8 ml of acetic acid and 2 ml of trifluoroacetic acid, heated to reflux for 4 h and stirred at room temperature overnight. Subsequently, the mixture is admixed with water and ethyl acetate, and the organic phase is washed with sodium hydrogencarbonate solution and water, dried over sodium sulfate and freed of solvent under reduced pressure. 110 mg (94% of theory) of the desired product are obtained as a colorless oil in 92% purity (HPLC).

HPLC (method 1): Rₜ=4.95 min.

MS (ESIpos): 292 (M+H)⁺

¹H NMR (300 MHz, CDCl₃): δ=1.65-2.15 (m, 8H), 3.38+3.60 (s, 2H), 4.64+4.77 (s, 2M, 7.36 (m, 3H), 7.44 (m, 3H), 7.55 (d, 2H), 8.19+8.33 (s, 1H).

### EXAMPLE 46A

### 7-(4-Fluorophenyl)-4-spirocyclopentyl-3,4-dihydro-2(1H)-isoquinolinecarbaldehyde

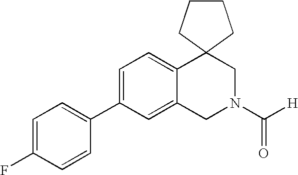

Analogously to the preparation of 7-phenyl-4-spirocyclopentyl-3,4-dihydro-2(1H)-isoquinolinecarbaldehyde, 210 mg (0.706 mmol) of [1-(4′-fluoro-1,1′-biphenyl-4-yl)cyclopentyl]methylformamide and 42.4 mg (0.523 mmol) of 37% formaldehyde solution in 16 ml of acetic acid and 4 ml of trifluoroacetic acid are used to obtain 170 mg (51% of theory) of the desired product as a colorless oil in 66% purity (HPLC).

LC-MS (method 3): Rₜ=2.93 min.

MS (ESIpos): 310 (M+H)⁺

¹H NMR (300 MHz, CDCl₃): δ=1.70-2.05 (m, 8H), 3.38+3.60 (s, 2H), 4.62+4.76 (s, 2H), 7.11 (m, 2H), 7.30-7.45 (m, 2H), 7.46-7.59 (m, 3H), 8.19+8.34 (s, 1H).

### EXAMPLE 47A

### 4-Spirocyclopentyl-7-[4-(trifluoromethyl)phenyl]-3,4-dihydro-2(1H)-isoquinolinecarbaldehyde

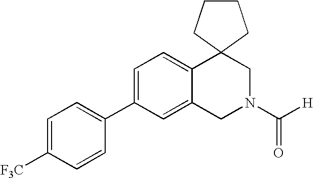

Analogously to the preparation of 7-phenyl-4-spirocyclopentyl-3,4-dihydro-2(1H)-isoquinolinecarbaldehyde, 259 mg (0.746 mmol) of [1-(4′-trifluoromethyl-1,1′-biphenyl-4-yl)cyclopentyl]methylformamide and 121 mg (1.491 mmol) of 37% formaldehyde solution in 24 ml of acetic acid and 6 ml of trifluoroacetic acid are used to obtain 237 mg (73% of theory) of the desired product as a colorless oil in 81% purity (HPLC).

HPLC (method 1): Rₜ=5.18 min.

MS (DCI): 377 (M+NH₄)⁺

¹H NMR (200 MHz, d₆-DMSO): δ=1.58-1.98 (m, 8H), 3.42+3.49 (s, 2H), 4.66+4.70 (s, 2H), 7.37-7.65 (m, 0.3H), 7.75-7.95 (m, 4H), 8.21+8.30 (s, 1H).

### EXAMPLE 48A

### 7-Bromo-4-spirocyclohexyl-3,4-dihydro-2(1H)-isoquinolinecarbaldehyde

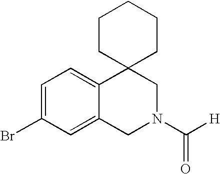

Analogously to the preparation of 7-phenyl-4-spirocyclopentyl-3,4-dihydro-2(1H)-isoquinolinecarbaldehyde, 1.23 g (2.95 mmol) of [1-(4-bromophenyl)cyclohexyl]-methylformamide and 479 mg (5.90 mmol) of 37% formaldehyde solution in 40 ml of acetic acid and 10 ml of trifluoroacetic acid are used to obtain 1.22 g (99% of theory) of the desired product as a colorless oil in 74% purity (HPLC).

LC-MS (method 3): Rₜ=2.84 min.

MS (ESIpos): 308 (M+H)⁺

¹H NMR (300 MHz, d₆-DMSO): δ=1.40-1.80 (m, 10H), 3.60+3.62 (s, 2H), 4.56+4.59 (s, 2H), 7.12-7.49 (m, 3H), 8.18+8.24 (s, 1H).

### EXAMPLE 49A

### 7-Phenyl-4-spirocyclohexyl-3,4-dihydro-2(1H)-isoquinolinecarbaldehyde

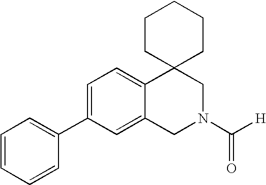

Analogously to the preparation of 7-phenyl-4-spirocyclopentyl-3,4-dihydro-2(1H)-isoquinolinecarbaldehyde, 265 mg (0.90 mmol) of [1-(1,1′-biphenyl-4-yl)cyclohexyl]methylformamide and 147 mg (1.81 mmol) of 37% formaldehyde solution in 24 ml of acetic acid and 6 ml of trifluoroacetic acid are used to obtain 235 mg (61% of theory) of the desired product as a colorless oil in 69% purity (HPLC).

LC-MS (method 3): Rₜ=3.01 min.

MS (ESIpos): 294 (M+H)⁺.

### EXAMPLE 50A

### 7-(4-Fluorophenyl)-4-spirocyclohexyl-3,4-dihydro-2(1H)-isoquinolinecarbaldehyde

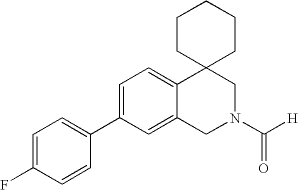

Analogously to the preparation of 7-phenyl-4-spirocyclopentyl-3,4-dihydro-2(1H)-isoquinolinecarbaldehyde, 680 mg (2.18 mmol) of [1-(4′-fluoro-1,1′-biphenyl-4-yl)cyclohexyl]methylformamide and 350 mg (4.37 mmol) of 37% formaldehyde solution in 40 ml of acetic acid and 10 ml of trifluoroacetic acid are used to obtain 770 mg (99% of theory) of the desired product as a colorless oil in 91% purity (HPLC).

HPLC (method 1): Rₜ=5.20 min.

MS (DCI): 341 (M+NH₄)⁺

¹H NMR (300 MHz, CDCl₃): δ=1.25-1.85 (m, 10H), 3.65+3.85 (s, 2H), 4.66+4.78 (s, 2H), 7.12 (m, 2H), 7.26 (m, 2H), 7.40-7.59 (m, 3H), 8.23+8.39 (s, 1H).

### EXAMPLE 51A

### 4-Spirocyclohexyl-7-[4-(trifluoromethyl)phenyl]-3,4-dihydro-2(1H)-isoquinolinecarbaldehyde

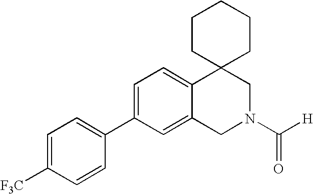

Analogously to the preparation of 7-phenyl-4-spirocyclopentyl 1 isoquinolinecarbaldehyde, 212 mg (0.59 mmol) of [1-(4′-trifluoromethyl-1,1′-biphenyl-4-yl)cyclohexyl]methylformamide and 95 mg (1.17 mmol) of 37% formaldehyde solution in 12 ml of acetic acid and 3 ml of trifluoroacetic acid are used to obtain 218 mg (94% of theory) of the desired product as a colorless oil in 94% purity (HPLC).

HPLC (method 1): Rₜ=4.38 min.

MS (ESIpos): 374 (M+H)⁺

¹H NMR (300 MHz, CDCl₃): δ=1.25-1.95 (m, 10H), 3.61+3.83 (s, 2H), 4.63+4.78 (s, 2H), 7.27-7.35 (m, 1H), 7.42-7.55 (m, 2H), 7.62-7.72 (m, 4H), 8.20+8.34 (s, 1H).

### EXAMPLE 52A

### 7-Methoxy-4-(2-phenylethyl)-3,4-dihydro-2(1H)-isoquinolinecarbaldehyde

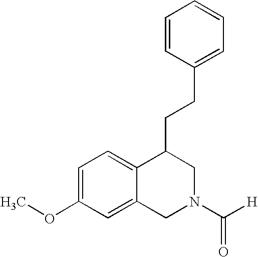

A mixture of 3.60 g (12.70 mmol) of 2-(4-methoxyphenyl)-4-phenylbutylformamide and 2.18 g (25.41 mmol) of formaldehyde solution is admixed with 150 ml of trifluoroacetic acid/acetic acid mixture (2:8). The mixture is heated to reflux for 2 h. Subsequently, the mixture is freed of solvent under reduced pressure and admixed with water and ethyl acetate. The organic phase is washed once with water and once with saturated sodium chloride solution, dried over sodium sulfate and freed of solvent under reduced pressure. Purification is effected by preparative HPLC. 2.35 g (62% of theory) of the desired product are obtained in 83% purity.

HPLC (method 1): Rₜ=4.65 min.

MS (DCI): 313 (M+NH₄)⁺

¹H NMR (300 MHz, CDCl₃): δ=2.60-2.94 (m, 4H), 3.43-3.70 (m, 2H), 3.78 (s, 3H), 4.29-4.56 (m, 2H), 5.01 (d, 1H), 6.54-6.69 (m, 1H), 6.72-6.83 (m, 1H), 7.01-7.09 (m, 2H), 7.12-7.36 (m, 4H), 8.12 (s, 1H).

### EXAMPLE 53A

### 7-Methoxy-1-methyl-1,2,3,4-tetrahydroisoquinoline

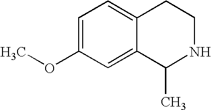

2.80 g (15.98 mmol) of 7-methoxy-1-methyl-3,4-dihydroisoquinoline in 140 ml of methanol are admixed at room temperature with 665 mg (17.58 mmol) of sodium borohydride and stirred for 1 hour. Subsequently, the mixture is admixed with water and ethyl acetate, the aqueous phase is extracted twice with ethyl acetate and the combined organic extracts are dried over sodium sulfate. After the solvent has been removed under reduced pressure, 2.70 g (95% of theory) of the desired product are obtained.

MS (ESIpos): m/z 178 (M+H)⁺

¹H NMR (300 MHz, CDCl₃): δ=1.43 (d, 2H), 2.70 (m, 1H), 2.96 (m, 1H), 3.23 (dt, 1H), 3.48 (m, 1H), 3.78 (s, 3H), 4.05 (m, 1H), 6.70 (m, 2H), 6.99 (d, 1H).

### EXAMPLE 54A

### 7-(Benzyloxy)-1,4,4-trimethyl-1,2,3,4-tetrahydroisoquinoline

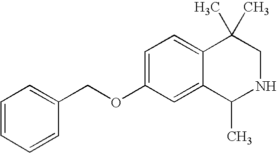

2.20 g (7.11 mmol) of 7-(benzyloxy)-1,4,4-trimethyl-3,4-dihydro-2(1H)-isoquinolinecarbaldehyde are dissolved in 30 ml of ethanol and admixed at room temperature with 22 ml of 2 molar sodium hydroxide solution. The mixture is boiled under reflux overnight. For workup, the mixture is cooled and diluted with methylene chloride and water. The organic phase is washed twice more with water and saturated sodium chloride solution, dried over sodium sulfate and freed of solvent under reduced pressure. 1.65 g (83% of theory) of the desired product are obtained in 90% purity.

HPLC (method 3): Rₜ=1.94 min.

MS (ESIpos): m/z=282 (M+H)⁺

¹H NMR (300 MHz, CDCl₃): δ=1.22 (s, 3H), 1.26 (s, 3H), 1.44 (d, 3H), 1.66 (s, 1H), 2.84 (dd, 2H), 5.02 (s, 2H), 6.69-6.85 (m, 2H), 7.20-7.46 (m, 6H).

### EXAMPLE 55A

### 7-(Benzyloxy)-4-spirocyclobutyl-1,2,3,4-tetrahydroisoquinoline

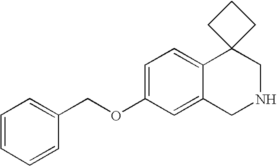

3.10 g (10.08 mmol) of 7-(benzyloxy)-4-spirocyclobutyl-3,4-dihydro-2(1H)-isoquinolinecarbaldehyde are dissolved in 30 ml of ethanol and admixed at room temperature with 32 ml of 2 molar sodium hydroxide solution. The mixture is boiled under reflux overnight. For workup, the mixture is cooled and diluted with ethyl acetate and water. The organic phase is washed with saturated sodium hydrogencarbonate solution, dried over sodium sulfate and freed of solvent under reduced pressure. 2.70 g (79% of theory) of the desired product are obtained in 82% purity.

HPLC (method 1): Rₜ=4.21 min.

MS (DCI): m/z=297 (M+NH₄)⁺

¹H NMR (300 MHz, CDCl₃): δ=1.62 (s, 1H), 1.94-2.08 (m, 4H), 2.24-2.37 (m, 2H), 3.12 (s, 2H), 3.95 (s, 2H), 5.02 (s, 2H), 6.53-6.92 (m, 2H), 7.24-7.52 (m, 6H).

### EXAMPLE 56A

### 7-(Benzyloxy)-4-spirocyclopentyl-1,2,3,4-tetrahydroisoquinoline

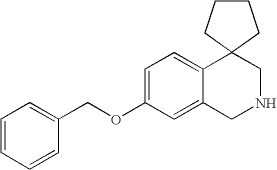

20.50 g (63.78 mmol) of 7-(benzyloxy)-4-spirocyclopentyl-3,4-dihydro-2(1H)-isoquinolinecarbaldehyde are dissolved in 200 ml of ethanol and admixed at room temperature with 191 ml (3882.68 mmol) of 2 molar sodium hydroxide solution. The mixture is boiled under reflux. For workup, the mixture is cooled and diluted with methylene chloride and water. The aqueous phase is extracted three times more with methylene chloride. The combined organic phases are combined and washed with saturated sodium hydrogencarbonate solution, dried over sodium sulfate and freed of solvent under reduced pressure. 19.47 g (76% of theory) of the desired product are obtained in 73% purity.

HPLC (method 1): Rₜ=4.45 min.

MS (ESIpos): m/z=294 (M+H)⁺

¹H NMR (200 MHz, CDCl₃): δ=1.60-1.94 (m, 9H), 2.86 (s, 2H), 3.99 (s, 2H), 5.02 (s, 2H), 6.50-6.89 (m, 2H), 7.10-7.49 (m, 6H).

### EXAMPLE 57A

### 7-(Benzyloxy)-4-spirocyclohexyl-1,2,3,4-tetrahydroisoquinoline

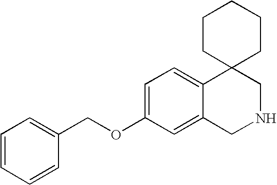

3.10 g (9.24 mmol) of 7-(benzyloxy)-4-spirocyclohexyl-3,4-dihydro-2(1H)-isoquinolinecarbaldehyde are dissolved in 100 ml of ethanol and admixed at room temperature with 28 ml (55.45 mmol) of 2 molar sodium hydroxide solution. The mixture is boiled under reflux. For workup, the mixture is cooled and diluted with methylene chloride and water. The aqueous phase is extracted three times more with methylene chloride. The combined organic phases are combined and washed with saturated sodium hydrogencarbonate solution, dried over sodium sulfate and freed of solvent under reduced pressure. 2.83 g (86% of theory) of the desired product are obtained in 86% purity.

HPLC (method 1): Rₜ=4.46 min.

MS (ESIpos): m/z=308 (M+H)⁺

¹H NMR (200 MHz, CDCl₃): δ=1.22-1.98 (m, 11H), 3.08 (s, 2H), 3.95 (s, 2H), 5.01 (s, 2H), 6.52-6.91 (m, 2H), 7.17-7.48 (m, 6H).

### EXAMPLE 58A

### 7-Phenyl-4-spirocyclopentyl-1,2,3,4-tetrahydroisoquinoline

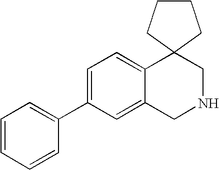

A solution of 107 mg (0.37 mmol) of 7-phenyl-4-spirocyclopentyl-3,4-dihydro-2(1H)-isoquinolinecarbaldehyde in 10 ml of ethanol is admixed at 0° C. with 2.2 ml (2.20 mmol) of 1 M sodium hydroxide and, after the addition, stirred at 70° C. for 6 hours. After cooling to room temperature, the mixture is admixed with water and ethyl acetate, the aqueous phase is extracted with ethyl acetate, and the combined organic phases are washed with saturated sodium hydrogencarbonate solution and dried over sodium sulfate. After the solvent has been removed under reduced pressure, 52 mg (54% of theory) of the desired product are obtained in 98% purity.

HPLC (method 1): Rₜ=4.30 min.

MS (ESIpos): 264 (M+H)⁺

¹H NMR (200 MHz, CDCl₃): δ=1.65-1.98 (m, 8H), 2.40 (broad, s, 1H), 2.89 (s, 2H), 4.09 (s, 2H), 7.20 (m, 1H), 7.30-7.49 (m, 5H), 7.51-7.61 (m, 2H).

### EXAMPLE 59A

### 7-(4-Fluorophenyl)-4-spirocyclopentyl-1,2,3,4-tetrahydroisoquinoline

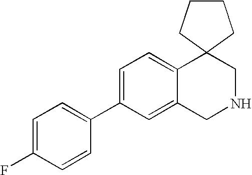

Analogously to the preparation of 7-phenyl-4-spirocyclopentyl-1,2,3,4-tetrahydroisoquinoline, 170 mg (0.55 mmol) of 7-(4-fluorophenyl)-4-spirocyclopentyl-3,4-dihydro-2(1H)-isoquinolinecarbaldehyde and 3.0 ml (3.30 mmol) of 1 M sodium hydroxide solution in 10 ml of ethanol are used to obtain 101 mg (65% of theory) of the desired product in 100% purity.

HPLC (method 1): Rₜ=4.36 min.

MS (ESIpos): 282 (M+H)⁺

¹H NMR (200 MHz, CDCl₃): δ=1.68-1.98 (m, 8H), 2.76 (s, 1H), 2.88 (s, 2H), 4.06 (s, 2H), 7.06-7.13 (m, 3H), 7.33-7.36 (m, 1H), 7.48-7.56 (m, 3H).

### EXAMPLE 60A

### 4-Spirocyclopentyl-7-[4-(trifluoromethyl)phenyl]-1,2,3,4-tetrahydroisoquinoline

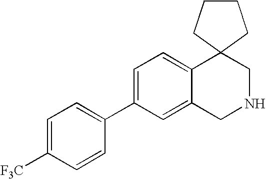

Analogously to the preparation of 7-phenyl-4-spirocyclopentyl-1,2,3,4-tetrahydroisoquinoline, 295 mg (0.82 mmol) of 7-(4-trifluoromethyl)phenyl]-4-spirocyclopentyl-3,4-dihydro-2(1H)-isoquinolinecarbaldehyde and 4.93 ml (4.93 mmol) of 1 M sodium hydroxide solution in 6 ml of ethanol are used to obtain 380 mg (96% of theory) of the desired product in 92% purity.

LC-MS (method 3): Rₜ=1.99 min.

MS (ESIpos): 332 (M+H)⁺.

### EXAMPLE 61A

### 7-Bromo-4-spirocyclohexyl-1,2,3,4-tetrahydroisoquinoline

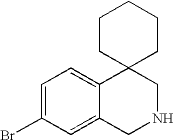

Analogously to the preparation of 7-phenyl-4-spirocyclopentyl-1,2,3,4-tetrahydroisoquinoline, 1.13 g (2.71 mmol) of 7-bromo-4-spirocyclohexyl-3,4-dihydro-2(1H)-isoquinolinecarbaldehyde and 8.14 ml (16.28 mmol) of 2 M sodium hydroxide solution in 30 ml of ethanol are used to obtain, after purification by HPLC, 390 mg (36% of theory) of the desired product in 71% purity.

HPLC (method 1): Rₜ=4.17 min.

MS (ESIpos): 280 (M+H)⁺

¹H NMR (200 MHz, CDCl₃): δ=1.23-1.85 (m, 11H), 3.07 (s, 1H), 3.94 (s, 2H), 7.12 (m, 1H), 7.26 (m, 2H).

### EXAMPLE 62A

### 7-Phenyl-4-spirocyclohexyl-1,2,3,4-tetrahydroisoquinoline

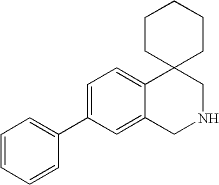

Analogously to the preparation of 7-phenyl-4-spirocyclopentyl-1,2,3,4-tetrahydroisoquinoline, 230 mg (0.53 mmol) of 7-phenyl-4-spirocyclopentyl-3,4-dihydro-2(1H)-isoquinolinecarbaldehyde and 3.7 ml (3.70 mmol) of 1 M sodium hydroxide solution in 6 ml of ethanol are used to obtain, after purification by HPLC, 40 mg (27% of theory) of the desired product in 98% purity.

LC-MS (method 3): Rₜ=1.94 min.

MS (ESIpos): 278 (M+H)⁺.

### EXAMPLE 63A

### 7-(4-Fluorophenyl)-4-spirocyclohexylpentyl-1,2,3,4-tetrahydroisoquinoline

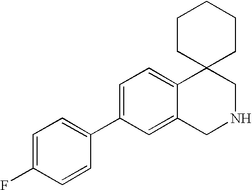

Analogously to the preparation of 7-phenyl-4-spirocyclopentyl-1,2,3,4-tetrahydroisoquinoline, 700 mg (2.16 mmol) of 7-(4-fluorophenyl)-4-spirocyclohexyl-3,4-dihydro-2(1H)-isoquinolinecarbaldehyde and 13.0 ml (13.00 mmol) of 1 M sodium hydroxide solution in 10 ml of ethanol are used to obtain, after purification using silica gel, 140 mg (22% of theory) of the desired product in 91% purity.

HPLC (method 1): Rₜ=4.50 min.

MS (ESIpos): 296 (M+H)⁺

¹H NMR (200 MHz, CDCl₃): δ=1.23-1.90 (m, 11H), 3.12 (s, 1H), 4.06 (s, 2H), 7.02-7.19 (m, 3H), 7.32-7.60 (m, 4H).

### EXAMPLE 64A

### 4-Spirocyclohexyl-7-[4-(trifluoromethyl)phenyl]-1,2,3,4-tetrahydroisoquinoline

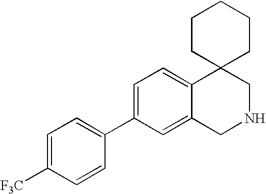

Analogously to the preparation of 7-phenyl-4-spirocyclopentyl-1,2,3,4-tetrahydroisoquinoline, 212 mg (0.57 mmol) of 7-[4-(trifluoromethyl)phenyl]-4-spirocyclopentyl-3,4-dihydro-2(1H)-isoquinolinecarbaldehyde and 3.40 ml (3.40 mmol) of 1 M sodium hydroxide solution in 10 ml of ethanol are used to obtain 195 mg (99% of theory) of the desired product in 69% purity.

LC-MS (method 3): Rₜ=2.04 min.

MS (ESIpos): 346 (M+H)⁺.

### EXAMPLE 65A

### 7-Methoxy-4-(2-phenylethyl)-1,2,3,4-tetrahydroisoquinoline

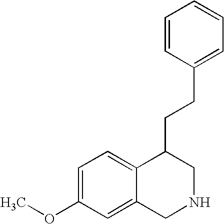

Analogously to the preparation of 7-phenyl-4-spirocyclopentyl-1,2,3,4-tetrahydroisoquinoline, 2.30 g (7.79 mmol) of 7-methoxy-4-(2-phenylethyl)-3,4-dihydro-2(1H)-isoquinolinecarbaldehyde and 23.6 ml of 2 N sodium hydroxide solution in 100 ml of ethanol (4 hours at 60° C.) are used to obtain 1.9 g (92% of theory) of the desired product in 62% purity.

HPLC (method 1): Rₜ=4.18 min.

MS (ESIpos): 268 (M+H)⁺

¹H NMR (200 MHz, CDCl₃): δ=1.87-2.10 (m, 2H), 2.58-2.82 (m, 4H), 3.11 (q, 2H), 3.77 (s, 3H, 3.97 (s, 2H), 6.49-6.58 (m, 1H), 6.68-6.77 (m, 1H), 7.12-7.36 (m, 6H).

### EXAMPLE 66A

### Ethyl(4-{[7-(hydroxy)-4-methyl-3,4-dihydro-2(1H)-isoquinolinyl]sulfonyl}-2-methylphenoxy)acetate

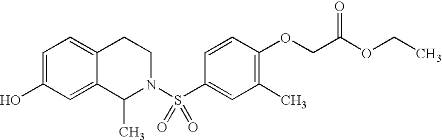

A solution of 1.90 g (4.38 mmol) of ethyl(4-{[7-(methoxy)-4-methyl-3,4-dihydro-2(1H)-isoquinolinyl]sulfonyl}-2-methylphenoxy)acetate (Example 2) in 34 ml of methylene chloride is admixed at −70° C. with 0.70 ml (7.45 mmol) of a 1.7 M boron tribromide solution in methylene chloride and subsequently stirred at this temperature for 1 hour. Another 1.86 g (7.45 mmol) of 99% boron tribromide are added, the reaction mixture is warmed to 0° C. and stirred at this temperature for 1 hour. Subsequently, the mixture is admixed with 4 ml of methanol and diluted with water and ethyl acetate, the aqueous phase is extracted with ethyl acetate and the combined organic extracts are dried over sodium sulfate. After concentration under reduced pressure and purification of a crude product on silica gel (eluent:cyclohexane→3:2 cyclohexane/ethyl acetate), 1.80 g (98% of theory) of the desired product were obtained.

HPLC (method 1): Rₜ=4.56 min.

MS (ESIpos): 420 (M+H)⁺

¹H NMR (300 MHz, CDCl₃): δ=1.28 (t, 3H), 1.46 (d, 3H), 2.23 (s, 3H), 2.57 (m, 2H), 3.39 (ddd, 1H), 3.81 (dddd, 1H), 4.25 (q, 2H), 4.64 (s, 2H), 4.72 (s, 1H), 5.04 (q, 1H), 6.51 (d, 1H), 6.60 (dd, 1H), 6.64 (d, 1H), 6.84 (d, 1H), 7.55 (s, 1H), 7.56 (dd, 1H).

### EXAMPLE 67A

### Ethyl(4-{[7-(hydroxy)-1,4,4-trimethyl-3,4-dihydro-2(1H)-isoquinolinyl]sulfonyl}-2-methylphenoxy)acetate

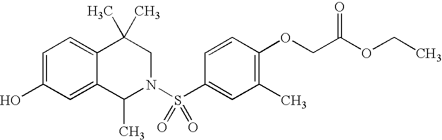

Under argon, 2.00 g (3.72 mmol) of ethyl(4-{[7-(benzyloxy)-1,4,4-trimethyl-3,4-dihydro-2(1H)-isoquinolinyl]sulfonyl}-2-methylphenoxy)acetate (Example 3) are taken up in 50 ml of ethanol and 10 ml of tetrahydrofuran. To this are added 2.35 g (37.2 mmol) of ammonium formate, the mixture is stirred briefly and 0.30 g of 10% palladium on carbon is subsequently added. The mixture is stirred at room temperature overnight. For workup, the mixture is diluted with methylene chloride and filtered with suction through kieselguhr. The filtrate is admixed with water, the aqueous phase is extracted with methylene chloride, and the combined organic extracts are washed with saturated sodium chloride solution, dried over sodium sulfate and freed of solvent under reduced pressure. 1.68 g (99% of theory) of the desired products are obtained in 99% purity.

LC-MS (method 3): Rₜ=4.79 min.

MS (ESIpos): 448 (M+H)⁺

¹H NMR (300 MHz, CDCl₃): δ=1.23-1.33 (m, 9H), 2.32 (s, 3H), 3.11 (d, 1H), 3.47 (d, 1H), 4.27 (q, 2H), 4.70 (s, 2H), 4.78 (s, 1H), 5.04 (q, 1H), 6.47 (d, 1H), 6.65-6.75 (m, 2H), 7.14 (d, 1H), 7.62-7.71 (m, 2H).

### EXAMPLE 68A

### Ethyl {4-[(7-hydroxy-4-spirocyclobutyl-3,4-dihydro-2(1H)-isoquinolinyl)sulfonyl]-2-methylphenoxy}acetate

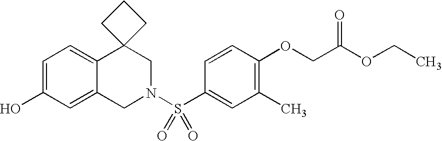

Under argon, 0.77 g (1.44 mmol) of ethyl(4-{[7-(benzyloxy)-4-spirocyclobutyl-3,4-dihydro-2(1H)-isoquinolinyl]sulfonyl}-2-methylphenoxy)acetate (Example 4) is taken up in 10 ml of ethanol and 2 ml of tetrahydrofuran. To this is added 0.91 g (14.42 mmol) of ammonium formate, the mixture is stirred briefly and 0.06 g of 10% palladium on carbon is subsequently added. The mixture is stirred at room temperature overnight. For workup, the mixture is filtered with suction through Kieselguhr. The filtrate is admixed with water, ethyl acetate and saturated sodium chloride solution, and the aqueous phase is extracted with ethyl acetate. The organic phase is dried over sodium sulfate and freed of solvent under reduced pressure. 0.69 g (84% of theory) of the desired product is obtained in 78% purity.

LC-MS (method 3): Rₜ=2.83 min.

MS (ESIpos): m/z=446 (M+H)⁺

¹H NMR (200 MHz, CDCl₃): δ=1.30 (t, 3H), 1.90-2.20 (m, 4H), 2.20-2.35 (m, 2H), 2.36 (s, 3H), 3.26 (s, 2H), 4.10 (s, 2H), 4.28 (q, 2H), 4.70 (s, 2H), 6.45 (d, 1H), 6.73 (dd, 1H), 6.79 (d, 1H), 7.40 (d, 1H), 7.55 (d, 1H), 7.57 (d, 1H).

### EXAMPLE 69A

### Ethyl {4-[(7-hydroxy-4-spirocyclopentyl-3,4-dihydro-2(1H)-isoquinolinyl)sulfonyl]-2-methylphenoxy}acetate

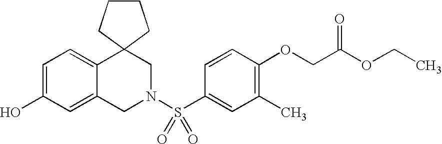

Under argon, 1.65 g (3.00 mmol) of ethyl-(4-{[7-(benzyloxy)-4-spirocyclopentyl-3,4-dihydro-2(1H)-isoquinolinyl]sulfonyl}-2-methylphenoxy)acetate (Example 5) are taken up in 20 ml of ethanol and 4 ml of tetrahydrofuran. To this are added 1.89 g (30.02 mmol) of ammonium formate, the mixture is stirred briefly and 0.09 g of 10% palladium on carbon is subsequently added. The mixture is stirred at room temperature overnight. For workup, the mixture is filtered with suction with Kieselguhr. The filtrate is admixed with water, ethyl acetate and saturated sodium chloride solution, and the aqueous phase is extracted with ethyl acetate. The organic phase is dried over sodium sulfate and freed of solvent under reduced pressure. The residue is purified using silica gel 60 (eluent: 3:1 cyclohexane/ethyl acetate). The clean fractions are combined and freed of solvent under reduced pressure. 1.33 g (96% of theory) of the desired product are obtained in 98% purity.

HPLC (method 1): Rₜ=4.98 min.

MS (ESIpos): m/z=460 (M+H)⁺

¹H NMR (200 MHz, CDCl₃): δ=1.30 (t, 3H), 1.82 (s, 8H), 2.35 (s, 3H), 2.97 (s, 2H), 4.12 (s, 2H), 4.27 (q, 2H), 4.71 (s, 2H), 6.44 (d, 1H), 6.68 (dd, 1H), 6.78 (d, 1H), 7.13 (d 1H), 7.63, (s, 1H), 7.64 (d, 1H).

### EXAMPLE 70A

### Ethyl {4-[(7-hydroxy-4-spirocyclohexyl-3,4-dihydro-2(1H)-isoquinolinyl)sulfonyl]-2-methylphenoxy}acetate

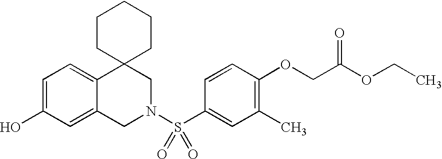

Under argon, 3.70 g (6.56 mmol) of ethyl(4-{[7-(benzyloxy)-4-spirocyclohexyl-3,4-dihydro-2(1H)-isoquinolinyl]sulfonyl}-2-methylphenoxy)acetate (Example 6) are taken up in 40 ml of ethanol and 20 ml of tetrahydrofuran. 4.14 g. (65.64 mmol) of ammonium formate are added, the mixture is stirred briefly and 0.16 g of 10% palladium on carbon is subsequently added. The mixture is stirred at room temperature overnight. For workup, the mixture is filtered with suction through Kieselguhr. The filtrate is admixed with water, ethyl acetate and saturated sodium chloride solution, and the aqueous phase is extracted with ethyl acetate. The organic phase is dried over sodium sulfate and freed of solvent under reduced pressure. 3.00 g (94% of theory) of the desired product are obtained in 98% purity.

HPLC (method 1): Rₜ=5.07 min.

MS (ESIpos): m/z=474 (M+H)⁺

¹HNMR (200 MHz, CDCl₃): δ=1.30 (t, 3H), 1.52-1.85 (m, 10H), 2.09 (s, 1H), 2.37 (s, 3H), 3.22 (s, 2H), 4.12 (s, 2H), 4.32 (q, 2H), 4.75 (s, 2H), 6.50 (d, 1H), 6.72 (dd, 1H), 6.82 (d, 1H), 7.25 (d, 1H), 7.68 (s, 1H), 7.70 (d, 1H).

### EXAMPLE 71A

### Ethyl(4-{[7-hydroxy-4-(2-phenylethyl)-3,4-dihydro-2(1H)-isoquinolinyl]sulfonyl}-2-methylphenoxy)acetate

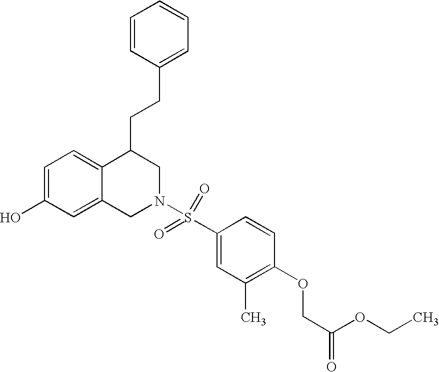

Under argon supply, 1.78 g (3.40 mmol) of ethyl(4-{[7-methoxy-4-(2-phenylethyl)-3,4-dihydro-2(1H)-isoquinolinyl]sulfonyl}-2-methylphenoxy)acetate (Example 17) are dissolved in 35.6 ml of dichloromethane. The solution is cooled to −78° C. and admixed with 7.81 ml (5.78 mmol) of a 0.74 molar boron tribromide solution in dichloromethane. The mixture is stirred under cold conditions for a further hour, then at 0° C. for one hour and at room temperature for one hour. For workup, the mixture is cooled again to 0° C. and diluted with ethyl acetate and water. The aqueous phase is extracted twice more with ethyl acetate. The combined organic phases are dried over sodium sulfate, filtered and freed of solvent under reduced pressure. The residue is purified by preparative HPLC. The clean fractions are combined and freed of solvent under reduced pressure. 1.46 g (84% of theory) of the desired product are obtained in 100% purity.

HPLC (method 1): Rₜ=5.03 min.

MS (ESIpos): m/z=510 (M+H)⁺

¹H NMR (300 MHz, CDCl₃): δ=1.32 (t, 3H), 1.84-1.97 (m, 1H), 2.00-2.06 (m, 1H), 2.32 (s, 31), 2.59-2.91 (m, 4H), 3.72-3.80 (m, 1H), 3.85 (d, 1H), 4.26 (q, 2H), 4.47 (d, 1H), 4.68 (s, 2H), 4.75 (s, 1H), 6.46-6.51 (m, 1H), 6.59-6.66 (m, 1H), 6.76 (d, 1H), 6.94 (d, 1H), 7.13-7.32 (m, 5H), 7.61-7.68 (m, 2H).

### EXAMPLE 72A

### Ethyl(2-methyl-{[7-{[(trifluoromethyl)sulfonyl]oxy}-4-methyl-3,4-dihydro-2(1H)-isoquinolinyl]sulfonyl}phenoxy)acetate

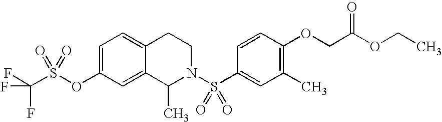

Under argon, 1.87 g (4.46 mmol) of ethyl {4-[(7-hydroxy-4-methyl-3,4-dihydro-2(1H)-isoquinolinyl)sulfonyl]-2-methylphenoxy}acetate (Example 66A) are dissolved in 25 ml of pyridine and admixed at 0° C. slowly for 2.52 g (8.92 mmol) of trifluoromethanesulfonic anhydride. The reaction mixture is allowed to come to room temperature and stirred overnight and admixed with water and ethyl acetate. The aqueous phase is extracted twice with ethyl acetate, and the combined organic phases are washed twice with 1 M hydrochloric acid, dried over sodium sulfate and freed of solvent under reduced pressure. 2.20 g (86% of theory) of the desired product are obtained in 96% purity.

HPLC (method 1): Rₜ=5.33 min.

MS (ESIpos): 552 (M+H)⁺

¹H NMR (300 MHz, CDCl₃): δ=1.28 (t, 3H), 1.46 (d, 3H), 2.23 (s, 3H), 2.62 (m, 2H), 3.37 (ddd, 1H), 3.90 (dddd, 1H), 4.25 (q, 2H), 4.73 (s, 2H), 5.14 (q, 1H), 6.63 (d, 1H), 6.96 (d, 1H), 7.03 (dd, 1H), 7.05 (d, 1H), 7.55 (s, 1H), 7.57 (dd, 1H).

### EXAMPLE 73A

### Ethyl(2-methyl-{[7-{[(trifluoromethyl)sulfonyl]oxy}-1,4,4-trimethyl-3,4-dihydro-2(1H)-isoquinolinyl]sulfonyl}phenoxy)acetate

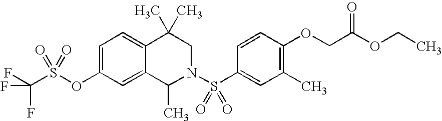

Under argon, 1.68 g (3.75 mmol) of ethyl {4-[(7-hydroxy-1,4,4-trimethyl-3,4-dihydro-2(1H)-isoquinolinyl)sulfonyl]-2-methylphenoxy}acetate (Example 67A) are dissolved in 20 ml of pyridine and admixed at 0° C. slowly with 2.12 g (7.51 mmol) of trifluoromethanesulfonic anhydride. The mixture is allowed to come to room temperature and stirred overnight, admixed once again at 0° C. with 0.42 g (1.52 mmol) of trifluoromethanesulfonic anhydride and stirred at room temperature for another 2 hours. The reaction mixture is admixed with water and ethyl acetate, the aqueous phase is extracted twice with ethyl acetate, and the combined organic phases are washed twice with 1 M hydrochloric acid, dried over sodium sulfate and freed of solvent under reduced pressure. 1.90 g (77% of theory) of the desired product are obtained in 89% purity.

HPLC (method 1): Rₜ=5.61 min.

MS (ESIpos): 580 (M+H)⁺

¹H NMR (300 MHz, CDCl₃): δ=1.25-1.33 (m, 9H), 2.33 (s, 3H), 3.12 (d, 1H), 3.52 (d, 1H), 4.27 (q, 2H), 4.69 (s, 2H), 5.11 (q, 1H), 6.74 (d, 1H), 6.91 (d, 1H), 7.09 (dd, 1H), 7.35 (d, 1H), 7.63-7.70 (m, 2H).

### EXAMPLE 74A

### Ethyl(2-methyl-{[4-spirocyclobutyl-7-{[(trifluoromethyl)sulfonyl]oxy}-3,4-dihydro-2(1H)-isoquinolinyl]sulfonyl}phenoxy)acetate

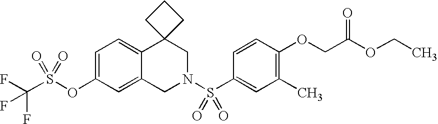

Under argon, 0.68 g (1.19 mmol) of ethyl {4-[(7-hydroxy-4-spirocyclobutyl-3,4-dihydro-2(1H)-isoquinolinyl)sulfonyl]-2-methylphenoxy}acetate (Example 68A) is dissolved in 50 ml of methylene chloride. To this is added dropwise 0.27 g (2.62 mmol) of triethylamine and the mixture is cooled to 0° C. Subsequently, 0.37 g (1.31 mmol) of trifluoromethanesulfonic anhydride is added dropwise. The mixture is allowed to come to room temperature and stirred overnight. The mixture is concentrated under reduced pressure and the residue purified using silica gel 60 (eluent: 3:1 cyclohexane/ethyl acetate). The clean fractions are combined and freed of solvent under reduced pressure. 0.49 g (71% of theory) of the desired product is obtained in 95% purity.

LC-MS (method 3): Rₜ=3.18 min.

MS (ESIpos): m/z=578 (M+H)⁺

¹H-NMR (300 MHz, CDCl₃): δ=1.30 (t, 3H), 2.04-2.21 (m, 6H), 2.36 (s, 3H), 3.31 (s, 2H), 4.18 (s, 2H), 4.27 (q, 2H), 4.71 (s, 2H), 6.80 (d, 1H, 6.92 (d, 1H), 7.16 (dd, 1H), 7.59-7.71 (m, 3H).

### EXAMPLE 75A

### Ethyl(2-methyl-{[4-spirocyclopentyl-7-{[(trifluoromethyl)sulfonyl]oxy}-3,4-dihydro-2(1H)-isoquinolinyl]sulfonyl}phenoxy)acetate

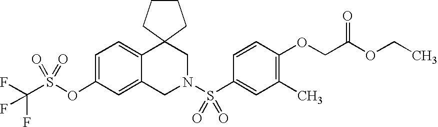

Under argon, 7.30 g (15.88 mmol) of ethyl {4-[(7-hydroxy-4-spirocyclopentyl-3,4dihydro-2(1H)-isoquinolinyl)sulfonyl]-2-methylphenoxy}acetate (Example 69A) is dissolved in 70 ml of methylene chloride. To this are added dropwise 3.54 g (34.95 mmol) of triethylamine and the mixture is cooled to 0° C. Subsequently, 4.93 g (17.47 mmol) of trifluoromethanesulfonic anhydride is added dropwise. The mixture is allowed to come to room temperature and stirred overnight. The mixture is concentrated under reduced pressure and the residue purified using silica gel 60 (eluent: methylene chloride). The clean fractions are combined and freed of solvent under reduced pressure. 4.90 g (52% of theory) of the desired product is obtained in 92% purity.

HPLC (method 2): Rₜ=5.69 min.

MS (ESIpos): m/z=592 (M+H)⁺

¹H NMR (200 MHz, CDCl₃): δ=1.30 (t, 3H), 1.72-1.99 (m, 8H), 2.36 (s, 3H), 2.99 (s, 2H), 4.19 (s, 2H), 4.28 (q, 2H), 4.71 (s, 2H), 6.79 (d, 1H), 6.91 (d, 1H), 7.09 (dd, 1H), 7.34 (d, 1H), 7.57-7.69 (m, 2H).

### EXAMPLE 76A

### Ethyl(2-methyl-{[4-spirocyclohexyl-7-{[(trifluoromethyl)sulfonyl]oxy}-3,4-dihydro-2(1H)-isoquinolinyl]sulfonyl}phenoxy)acetate

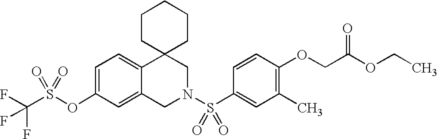

Under argon, 0.65 g (1.37 mmol) of ethyl {4-[(7-hydroxy-4-spirocyclohexyl-3,4-dihydro-2(1H)-isoquinolinyl)sulfonyl]-2-methylphenoxy}acetate (Example 70A) is dissolved in 5 ml of methylene chloride. To this is added dropwise 0.31 g (0.42 ml; 3.02 mmol) of triethylamine and the mixture is cooled to 0° C. Subsequently, 0.43 g (1.51 mmol) of trifluoromethanesulfonic anhydride is added dropwise. The mixture is allowed to come to room temperature and stirred overnight. The mixture is concentrated under reduced pressure and the residue purified using silica gel 60′ (eluent: methylene chloride). The clean fractions are combined and freed of solvent under reduced pressure. 0.83 g (97% of theory) of the desired product is obtained in 97% purity.

HPLC (method 1): Rₜ=5.84 min.

MS (ESIpos): m/z=606 (M+H)⁺

¹H NMR (300 MHz, CDCl₃): δ=1.30 (t, 3H), 1.54-1.88 (m, 10H), 2.36 (s, 3H), 3.25 (s, 2H), 4.19 (s, 2H), 4.27 (q, 2H), 4.71 (s, 2H), 6.80 (d, 1H), 6.92 (d, 1H), 7.10 (dd, 1H), 7.44 (d, 1H), 7.62-7.70 (m, 2H).

### EXAMPLE 77A

### Ethyl(2-methyl-4-{[4-(2-phenylethyl)-7-{[(trifluoromethyl)sulfonyl]oxy}-3,4-dihydro-2(1H)-isoquinolinyl]sulfonyl}phenoxy)acetate

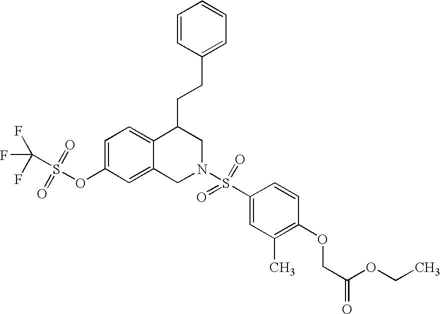

Under argon supply, 0.66 g (1.30 mmol) of ethyl(4-{[7-hydroxy-4-(2-phenylethyl)-3,4-dihydro-2(1H)-isoquinolinyl]sulfonyl}-2-methylphenoxy)acetate (Example 71A) is dissolved in 68.46 g (865.46 mmol) of pyridine and cooled to 0° C. 0.73 g (2.60 mmol) of trifluoromethanesulfonic anhydride is added dropwise and the mixture is subsequently stirred further at room temperature overnight. The mixture is cooled once again to 0° C., a further 0.07 g (0.26 mmol) of trifluoromethanesulfonic anhydride is added dropwise and the mixture is subsequently stirred further at room temperature for 30 minutes. Since reactant is still present, the mixture is cooled once more to 0° C. and admixed again with 0.07 g (0.26 mmol) of trifluoromethanesulfonic anhydride. After 30 minutes at 0° C., the mixture is warmed again to room temperature for 2 hours. For workup, the mixture is diluted with ethyl acetate and water and the phases are separated. The organic phase is washed once more with 1 N hydrochloric acid, dried over sodium sulfate, filtered and freed of solvent under reduced pressure. 1.02 g (quant.) of the desired product are obtained in 0.100% purity.

HPLC (method 1): Rₜ=5.87 min.

MS (ESIpos): m/z=641 (M+H)⁺

¹H NMR (200 MHz, CDCl₃): δ=1.30 (t, 3H), 1.82-2.02 (m, 1H), 2.04-2.23 (m, 1H), 2.34 (s, 3H), 2.55-2.97 (m, 4H), 3.87 (d, 2H), 4.27 (q, 2H), 4.59 (d, 1H), 4.70 (s, 2H), 6.71-6.83 (m, 1H), 6.90-7.00 (m, 1H), 7.02-7.09 (m, 1H), 7.15-7.37 (m, 6H), 7.59-7.70 (m, 2H).

### WORKING EXAMPLES

### Example 1

### Ethyl(4-{[7-bromo-4-spirocyclohexyl]-3,4-dihydro-2(1H)-isoquinolinyl]sulfonyl}-2-methylphenoxy)acetate

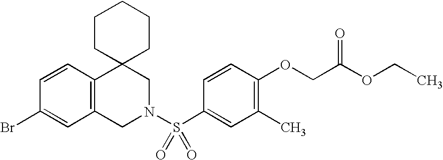

A solution of 407 mg (1.39 mmol) of ethyl 4-chlorosulfonyl-2-methylphenoxyacetate in 5 ml of tetrahydrofuran is added at 0° C. to a solution of 390 mg (1.39 mmol) of 7-bromo-4-spirocyclohexyl-1,2,3,4-tetrahydroisoquinoline, 310 mg (3.06 mmol) of triethylamine and 17 mg (0.139 mmol) of 4-dimethylaminopyridine in 5 ml of tetrahydrofuran and the mixture is stirred at room temperature overnight. Subsequently, the mixture is admixed with water and ethyl acetate, the aqueous phase is extracted with ethyl acetate and the combined organic phases are dried over sodium sulfate. After purification by preparative HPLC, 400 mg (54% of theory) of the desired product are obtained.

LC-MS (method 3): Rₜ=3.29 min.

MS (ESIpos): m/z=536 (M+H)⁺

¹H NMR (300 MHz, CDCl₃): δ=1.30 (t, 3H), 1.45-1.88 (m, 10H), 2.37 (s, 3H), 3.22 (s, 2H), 4.14 (s, 2H), 4.28 (q, 2H), 4.71 (s, 2H), 6.79 (d, 1H), 7.14 (d, 1H), 7.22 (d, 1H), 7.31 (dd, 1H), 7.64 (s, 1H), 7.65 (d, 1H).

### Example 2

### Ethyl(4-{[7-(methoxy)-4-methyl-3,4-dihydro-2(1H)-isoquinolinyl]sulfonyl}-2-methylphenoxy)acetate

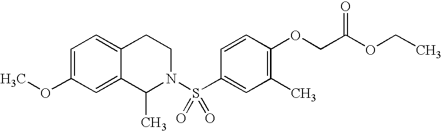

A solution of 3.138 g (10.72 mmol) of ethyl 4-chlorosulfonyl-2-methylphenoxyacetate in 65 ml of methylene chloride is added at 0° C. to a solution of 3.80 g (21.44 mmol) of 7-(methoxy)-4-methyl-1,2,3,4tetrahydroisoquinoline in 1 ml of methylene chloride and 65 ml of pyridine and the mixture is stirred at room temperature overnight. The mixture is worked up by diluting with ethyl acetate and water and extracting the aqueous phase. The combined organic phases are washed with 1 M hydrochloric acid, dried over sodium sulfate and freed of solvent under reduced pressure. After purification by preparative HPLC, 1.8 g (19% of theory) of the desired product are obtained.

HPLC (method 1): Rₜ=5.02 min.

MS (ESIpos): 434 (M+H)⁺

¹H NMR (300 MHz, CDCl₃): δ=1.28 (t, 3H), 1.48 (d, 3H), 2.23 (s, 3H), 2.57 (m, 2H), 3.41 (m, 1H), 3.60 (s, 3H), 3.79 (m, 1H), 4.25 (q, 2H), 5.08 (q, 1H), 6.58 (d, 1H), 6.68 (m, 2H); 6.89 (d, 1H), 7.55 (s, 1H), 7.57 (d, 1H).

### Example 3

### Ethyl(4-{[7-(benzyloxy)-1,4,4-trimethyl-3,4-dihydro-2(1H)-isoquinolinyl]sulfonyl}-2-methylphenoxy)acetate

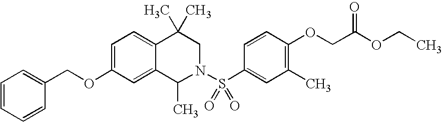

A solution of 2.14 g (7.32 mmol) of ethyl 4-chlorosulfonyl-2-methylphenoxyacetate in 15 ml of methylene chloride is added at 0° C. to a solution of 1.63 g (6.10 mmol) of 7-(benzyloxy)-1,4,4-trimethyl-1,2,3,4-tetrahydroisoquinoline in 5 ml of methylene chloride and 20 ml of pyridine and the mixture is stirred at room temperature overnight. The mixture is worked up by diluting with ethyl acetate and water and extracted. The combined organic phases are washed with 1 M hydrochloric acid, dried over sodium sulfate and freed of solvent under reduced pressure. After purification by preparative HPLC, 2.06 g (63% of theory) of the desired product are obtained.

HPLC (method 1): Rₜ=5.38 min.

MS (ESIpos): 538 (M+H)⁺

¹H NMR (300 MHz, CDCl₃): δ=1.23-1.33 (m, 9H), 2.32 (s, 3H), 3.12 (d, 1H), 3.47 (d, 1H), 4.27 (q, 2H), 4.70 (s, 2H), 5.01 (s, 2H), 5.06 (q, 1H), 6.60 (d, 1H), 6.72 (d, 1H), 6.84 (dd, 1H), 7.19 (d, 1H), 7.32-7.45 (m, 5H), 7.66 (s, 1H), 7.67 (m, 1H).

### Example 4

### Ethyl(4-{[7-(benzyloxy)-4-spirocyclobutyl-3,4-dihydro-2(1H)-isoquinolinyl]-sulfonyl}-2-methylphenoxy)acetate

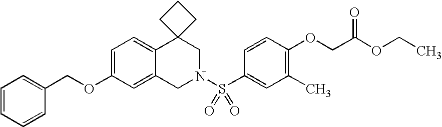

0.39 g (3.89 mmol) of triethylamine and a spatula-tip of 4-dimethylaminopyridine are added to a solution of 0.60 g (1.77 mmol) of 7-(benzyloxy)-4-spirocyclobutyl-1,2,3,4-tetrahydroisoquinoline in methylene chloride. The mixture is stirred at room temperature for a further 5 minutes and subsequently cooled to 0° C. 0.57 g (1.94 mmol) of ethyl 4-chlorosulfonyl-2-methylphenoxyacetate are dissolved in methylene chloride and added dropwise. The mixture is allowed to come to room temperature and stirred overnight. The mixture is worked up by diluting with ethyl acetate and water and extracted. The organic phase is dried over sodium sulfate and freed of solvent under reduced pressure. 0.90 g (83% of theory) of the desired product is obtained.

HPLC (method 2): Rₜ=5.44 min.

¹H NMR (300 MHz, CDCl₃): δ=1.19-1.35 (n, 6H), 1.91-2.40 (m, 6H), 3.28 (s, 2H), 4.13 (d, 2H), 4.27 (q, 2H), 4.69 (s, 2H), 5.01 (s, 2H), 6.75-6.81 (m, 2H), 7.24-7.49 (m, 7H), 7.64-7.71 (m, 2H).

### Example 5

### Ethyl(4-{[7-(benzyloxy)-4-spirocyclopentyl-3,4-dihydro-2(1H)-isoquinolinyl]-sulfonyl}-2-methylphenoxy)acetate

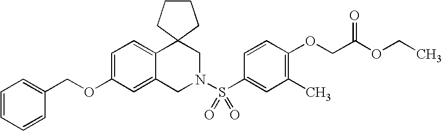

3.79 g (37.49 mmol) of triethylamine and 20.8 mg (0.17 mmol) of 4-dimethylaminopyridine are added to a solution of 5.00 g (17.04 mmol) of 7-(benzyloxy)-4-spirocyclopentyl-1,2,3,4-tetrahydroisoquinoline in 50 ml of methylene chloride. The mixture is stirred at room temperature for a further 5 minutes and subsequently cooled to 0° C. 5.49 g (18.75 mmol) of ethyl 4-chlorosulfonyl-2-methylphenoxyacetate are dissolved in 25 ml of methylene chloride and added dropwise. The mixture is allowed to come to room temperature and stirred. The mixture is worked up by diluting with ethyl acetate and water and extracted. The aqueous phase is extracted three times with ethyl acetate. The combined organic phases are dried over sodium sulfate and freed of solvent under reduced pressure. 8.80 g (94% of theory) of the desired product is obtained.

HPLC (method 1): Rₜ=5.51 min.

MS (ESIpos): m/z 550 (M+H)⁺

¹H NMR (300 MHz, CDCl₃): δ=1.52 (s, 3H), 1.74-1.89 (m, 8H), 2.34 (m, 3H), 4.15 (s, 2H), 4.27 (q, 2H), 4.69 (s, 2H), 5.00 (s, 2H), 6.58 (d, 1H), 6.75-6.86 (m, 2H), 7.17 (d, 1H), 7.27-7.43 (m, 5H), 7.59-7.67 (m, 2H).

### Example 6

### Ethyl(4-{[7-(benzyloxy)-4-spirocyclohexyl-3,4-dihydro-2(1H)-isoquinolinyl]-sulfonyl}-2-methylphenoxy)acetate

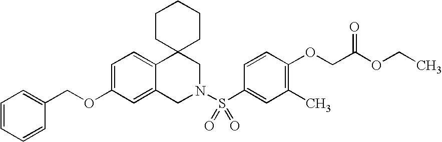

0.51 g (5.01 mmol) of triethylamine and 27.82 mg (0.23 mmol) of 4-dimethylaminopyridine are added to a solution of 0.70 g (2.28 mmol) of 7-(benzyloxy)-4-spirocyclohexyl-1,2,3,4-tetrahydroisoquinoline in 3 ml of methylene chloride. The mixture is stirred at room temperature for a further 5 minutes and 0.67 g (2.28 mmol) of ethyl 4-chlorosulfonyl-2-methylphenoxyacetate dissolved in 2 ml of methylene chloride is subsequently added dropwise. The mixture is left to stir at room temperature overnight. The mixture is worked up by diluting with methylene chloride and water and extracting. The aqueous phase is extracted three times with methylene chloride. The combined organic phases are dried over sodium sulfate and freed of solvent under reduced pressure. The residue is purified using silica gel 60 (eluent: methylene chloride). The clean fractions are combined and freed of solvent under reduced pressure. 0.82 g (64% of theory) of the desired product is obtained.

HPLC (method 2): Rₜ=5.94 min.

MS (ESIpos): m/z=564 (M+H)⁺

¹H NMR (300 MHz, CDCl₃): δ=1.29 (t, 3H), 1.52 (s, 4H), 1.57-1.84 (m, 6H), 2.35 (s, 3H), 3.22 (s, 2H), 4.15 (s, 2H), 4.27 (q, 2H), 4.70 (s, 2H), 5.00 (s, 2H), 6.59 (d, 1H), 6.75-6.87 (m, 2H), 7.27-7.42 (m, 6H), 7.63-7.70 (m, 2H).

### Example 7

### Ethyl(2-methyl-4-{[4-spirocyclopentyl-7-phenyl]-3,4-dihydro-2(1H)-isoquinolinyl]-sulfonyl}phenoxy)acetate

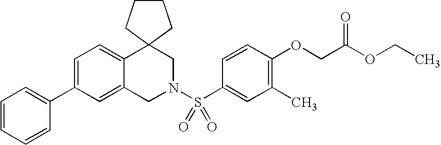

Analogously to the preparation of ethyl(4-{[7-benzyloxy)-4-spirocyclopentyl-3,4-dihydro-2(1H)-isoquinolinyl]sulfonyl}-2-methylphenoxy)acetate, 26 mg (0.10 mmol) of 7-phenyl-4-spirocyclopentyl-1,2,3,4-tetrahydroisoquinoline, 22 mg (0.22 mmol) of triethylamine, 1.2 mg (0.01 mmol) of 4-dimethylaminopyridine and 28.9 mg (0.10 mmol) of ethyl 4-chlorosulfonyl-2-methylphenoxyacetate in 6 ml of tetrahydrofuran are used to obtain 41 mg (63% of theory) of the desired product.

LC-MS (method 3): Rₜ=3.36 min.

MS (ESIpos): m/z=520 (M+H)⁺

¹H NMR (200 MHz, DMSO-d₆): δ=1.20 (t, 3H), 1.66-1.92 (m, 8H), 2.28 (s, 3H), 2.99 (s, ²H), 4.17 (q, 2H), 4.19 (s, 2H), 4.95 (s, 2H), 7.09 (d, 1H), 7.26-7.57 (m, 6H), 7.55-7.75 (m, 4H).

### Example 8

### Ethyl(2-methyl-{[7-[4-fluorophenyl]-4-spirocyclopentyl-3,4-dihydro-2(1H)-isoquinolinyl]sulfonyl}phenoxy)acetate

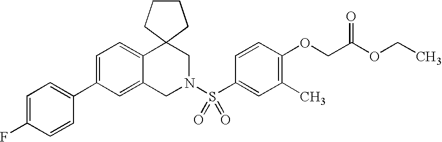

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 46 mg (0.16 mmol) of 7-(4-fluorophenyl-4-spirocyclopentyl-1,2,3,4-tetrahydroisoquinoline, 33 mg (0.33 mmol) of triethylamine, 2.0 mg (0.02 mmol) of 4-dimethylaminopyridine and 47.9 mg (0.16 mmol) of ethyl 4-chlorosulfonyl-2-methylphenoxyacetate in 6 ml of tetrahydrofuran are used to obtain 67 mg (51% of theory) of the desired product.

LC-MS (method 3): Rₜ=3.31 min.

MS (ESIpos): m/z=538 (M+H)⁺.

### Example 9

### Ethyl(2-methyl-4-{[4-spirocyclopentyl-7-[4-(trifluoromethyl)phenyl]-3,4-dihydro-2(1H)-isoquinolinyl]sulfonyl}phenoxy)acetate

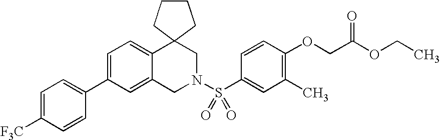

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 156 mg (0.46 mmol) of 7-[4-(trifluoromethyl)phenyl]-4-spirocyclopentyl-1,2,3,4-tetrahydroisoquinoline, 101 mg (1.00 mmol) of triethylamine, 5.6 mg (0.05 mmol) of 4-dimethylaminopyridine and 133.4 mg (0.46 mmol) of ethyl 4-chlorosulfonyl-2-methylphenoxyacetate in 6 ml of tetrahydrofuran are used to obtain 252 mg (81% of theory) of the desired product.

HPLC (method 1): Rₜ=6.10 min.

MS (ESIpos): m/z 588 (M+H)⁺

¹H NMR (300 MHz, DMSO-d₆): δ=1.20 (t, 3H), 1.71-1.92 (m, 8H), 2.29 (s, 3H), 2.97 (d, 2H), 4.12-4.25 (m, 4H), 4.94 (s, 2H), 7.10 (d, 1H), 7.45 (d, 1H), 7.51-7.54 (m, 1H), 7.59 (d, 1H), 7.62-7.71 (m, 2H), 7.83 (dd, 4H).

### Example 10

### Ethyl(2-methyl-4-{[7-phenyl-4-spirocyclohexyl]-3,4-dihydro-2(1H)-isoquinolinyl]-sulfonyl}phenoxy)acetate

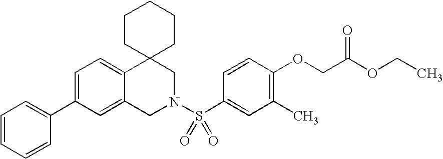

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 35 mg (0.10 mmol) of 7-phenyl-4-spirocyclohexyl-1,2,3,4-tetrahydroisoquinoline, 22.5 mg (0.22 mmol) of triethylamine, 1.2 mg (0.01 mmol) of 4-dimethylaminopyridine and 29.6 mg (0.10 mmol) of ethyl 4-chlorosulfonyl-2-methylphenoxyacetate in 6 ml of tetrahydrofuran are used to obtain 69 mg (94% of theory) of the desired product.

LC-MS (method 3): Rₜ=3.45 ml

MS (ESIpos): m/z=534 (M+H)⁺.

### Example 11

### Ethyl(2-methyl-4-{[7-[4-fluorophenyl]4-spirocyclohexyl-3,4-dihydro-2(1H)-isoquinolinyl]sulfonyl}phenoxy)acetate

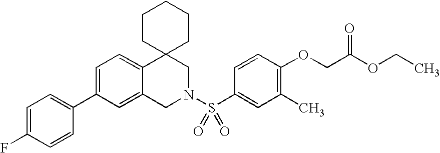

Analogously to the preparation of ethyl(4-{[7-(benzyloxy)-4-spirocyclopentyl-3,4-dihydro-2(1H)-isoquinolinyl]sulfonyl}-2-methylphenoxy)acetate; 70 mg (0.24 mmol) of 7-(4-fluorophenyl)-4-spirocyclohexyl-1,2,3,4-tetrahydroisoquinoline, 52.8 mg (0.52 mmol) of triethylamine, 2.9 mg (0.02 mmol) of 4-dimethylaminopyridine and 69.4 mg (0.24 mmol) of ethyl 4-chlorosulfonyl-2-methylphenoxyacetate in 6 ml of tetrahydrofuran are used to obtain 110 mg (72% of theory) of the desired product.

HPLC (method 1): Rₜ=6.20 min.

MS (ESIpos): m/z=552 (M+H)⁺

¹H NMR (300 MHz, DMSO-d₆): δ=1.30 (t, 3H), 1.44-1.84 (m, 10H), 2.30 (s, 3H), 3.21 (s, 2H), 4.12-4.26 (m, 4H), 4.95 (s, 2H), 7.06-7.16 (m, 1H), 7.20-7.33 (m, 2H), 7.38-7.58 (p, 3H), 7.61-7.75 (m, 4H).

### Example 12

### Ethyl(4-{[4-spirocyclohexyl-7-[4-(trifluoromethyl)phenyl]-3,4-dihydro-2(1H)-isoquinolinyl]sulfonyl}phenoxy)acetate

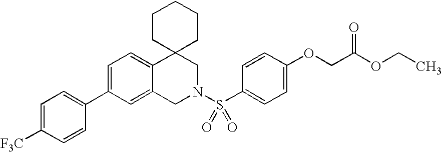

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 40 mg (0.12 mmol) of 7-[4-(trifluoromethyl)phenyl]-4-spirocyclohexyl-1,2,3,4-tetrahydroisoquinoline, 25.8 mg (0.25 mmol) of triethylamine, 1.4 mg (0.01 mmol) of 4-dimethylaminopyridine and 32.3 mg (0.12 mmol) of ethyl 4-chlorosulfonylphenoxyacetate in 6 ml of tetrahydrofuran are used to obtain 75 mg (99% of theory) of the desired product.

HPLC (method 2): Rₜ=6.02 min.

MS (ESIpos): m/z 588 (M+H)⁺

¹H NMR (200 MHz, DMSO-d₆): δ=1.21 (t, 3H), 1.37-1.86 (m, 10H), 3.22 (s, 2H), 4.09-4.26 (m, 4H), 4.94 (s, 2H), 7.19 (d, 2H), 7.57 (d, 2H), 7.75-7.95 (m, 5H), 8.09 (d, 2H).

### Example 13

### Ethyl(2-methyl-4-{[4-spirocyclohexyl-7-[4-(trifluoromethyl)phenyl]-3,4-dihydro-2(1H)-isoquinolinyl]sulfonyl}phenoxy)acetate

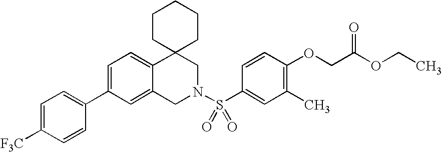

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 40 mg (0.12 mmol) of 7-[4-(trifluoromethyl)phenyl]-4-spirocyclohexyl-1,2,3,4-tetrahydroisoquinoline, 25.8 mg (0.25 mmol) of triethylamine, 1.4 mg (0.01 mmol) of 4-dimethylaminopyridine and 33.9 mg (0.12 mmol) of ethyl 4-chlorosulfonyl-2-methylphenoxyacetate in 6 ml of tetrahydrofuran are used to obtain 68 mg (98% of theory) of the desired product.

HPLC (method 1): Rₜ=6.21 min.

MS (ESIpos): m/z=602 (M+H)+

¹H NMR (200 MHz, DMSO-d₆): δ=1.21 (t, 3H), 1.44-1.83 (m, 10H), 2.94 (s, 3H), 3.22 (s, 2H), 4.08-4.25 (m, 4H), 4.96 (s, 2H), 6.58 (d, 1H), 7.11 (d, 1H), 7.49-7.62 (m, 2H), 7.65-7.95 (m, 5H), 8.03-8.14 (m, 1H).

### Example 14

### Ethyl(3-methyl-4-{[4-spirocyclohexyl-7-[4-(trifluoromethyl)phenyl]-3′,4-dihydro-2(1H)-isoquinolinyl]sulfonyl}phenoxy)acetate

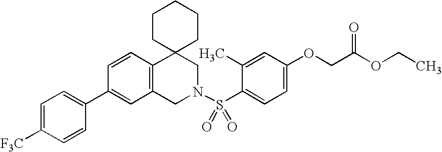

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 65 mg (0.19 mmol) of 7-[4-(trifluoromethyl)phenyl]-4-spirocyclohexyl-1,2,3,4-tetrahydroisoquinoline, 41.9 mg (0.41 mmol) of triethylamine, 2.3 mg (0.02 mmol) of 4-dimethylaminopyridine and 55.1 mg (0.19 mmol) of ethyl 4-chlorosulfonyl-3-methylphenoxyacetate in 6 ml of tetrahydrofuran are used to obtain 26 mg (23% of theory) of the desired product.

HPLC (method 1): Rₜ=6.27 min.

MS (ESIpos): m/z=0.602 (M+H)⁺

¹H NMR (200 MHz, DMSO-d₆): δ=1.21 (t, 3H), 1.44-1.83 (m, 10H), 2.56 (s, 3H), 3.40 (s, 2H), 4.18 (q, 2H), 4.37 (s, 2H), 4.92 (s, 2H), 7.04 (m, 2H), 7.60 (m, 3H), 7.75-7.93 (m, 5H).

### Example 15

### Ethyl(2,5-dimethyl-4-{[4-spirocyclohexyl-7-[4-(trifluoromethyl)phenyl]-3,4-dihydro-2(1H)-isoquinolinyl]sulfonyl}phenoxy)acetate

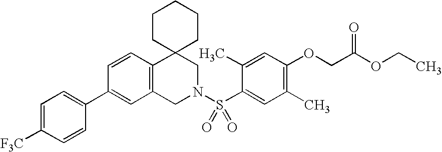

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 65 mg (0.19 mmol) of 7-[4-(trifluoromethyl)phenyl]-4-spirocyclohexyl-1,2,3,4-tetrahydroisoquinoline, 41.9 mg (0.41 mmol) of triethylamine, 2.3 mg (0.02 mmol) of 4-dimethylaminopyridine and 57.7 mg (0.19 mmol) of ethyl 4-chlorosulfonyl-2,6-dimethylphenoxyacetate in 6 ml of tetrahydrofuran are used to obtain 27 mg (23% of theory) of the desired product.

HPLC (method 1): Rₜ=6.55 min.

MS (ESIpos): m/z=616 (M+H)⁺

¹H NMR (200 MHz, DMSO-d₆): δ=1.20 (t, 3H), 1.25-1.83 (m, 10H), 2.25 (s, 3H), 2.54 (s, 3H), 3.37 (s, 2H), 4.18 (q, 2H), 4.34 (s, 2H), 4.93 (s, 2H), 6.96 (s, 1H), 7.60 (s, 3H), 7.72-7.95 (m, 5H).

### Example 16

### Ethyl(2,3-dimethyl-4-{[4-spirocyclohexyl-7-[4-(trifluoromethyl)phenyl]-3,4-dihydro-2(1H)-isoquinolinyl]sulfonyl}phenoxy)acetate

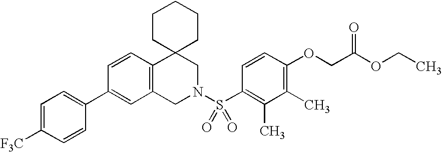

Analogously to the preparation of ethyl(4-{[7-(benzyloxy)-4-spirocyclopentyl-3,4-dihydro-2(1H)-isoquinolinyl]sulfonyl}-2-methylphenoxy)acetate, 64 mg (0.19 mmol) of 7-[4-(trifluoromethyl)phenyl]-4-spirocyclohexyl-1,2,3,4-tetrahydroisoquinoline, 41.3 mg (0.41 mmol) of triethylamine, 2.3 mg (0.02 mmol) of 4-dimethylaminopyridine and 56.8 mg (0.19 mmol) of ethyl 4-chlorosulfonyl-2,3-dimethylphenoxyacetate in 6 ml of tetrahydrofuran are used to obtain 29 mg (25% of theory) of the desired product.

HPLC (method 1): Rₜ=6.53 min.

MS (ESIpos): m/z 616 (M+H)⁺:

¹H NMR (200 MHz, DMSO-d₆): δ=1.22 (t, 3H), 1.35-1.83 (m, 10H), 2.19 (s, 3H), 2.52 (s, 3H), 3.42 (s, 2H), 4.18 (q, 2H), 4.38 (s, 2H), 4.94 (s, 2H), 6.98 (d, 1H), 7.49 (s, 3H), 7.74-7.94 (m, 5H).

### Example 17

### Ethyl(4-{[7-methoxy-4-(2-phenylethyl)-3,4-dihydro-2(1H)-isoquinolinyl]sulfonyl}-2-methylphenoxy)acetate

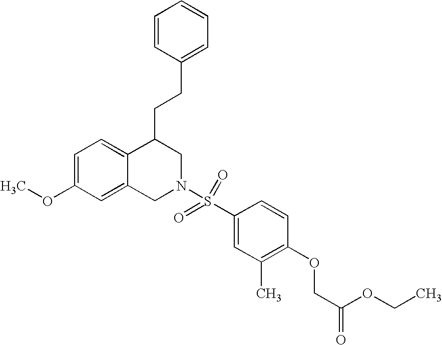

Under argon supply, 1.60 g (3.89 mmol) of 7-methoxy-4-(2-phenylethyl)-1,2,3,4-tetrahydroisoquinoline are dissolved in 16 ml of dichloromethane and 23.47 g (24.0 ml; 296.73 mmol) of pyridine and cooled to 0° C. 2.27 g (7.78 mmol) of ethyl 4-chlorosulfonyl-2-methylphenoxyacetate are dissolved in 8 ml of dichloromethane and the solution is added dropwise thereto. The mixture is allowed to come slowly to room temperature and left to stir overnight. For workup, the mixture is diluted with ethyl acetate and water and the phases are separated. The organic phase is washed twice more with 1 N hydrochloric acid, dried over sodium sulfate, filtered and freed of solvent under reduced pressure. The residue is purified by preparative HPLC in 2 batches. The clean fractions are combined and freed of solvent under reduced pressure. 1.78 g (87% of theory) of the desired product are obtained.

HPLC (method 1): Rₜ=5.50 min.

MS (ESIpos): m/z=524 (M+H)⁺

¹H NMR (200 MHz, CDCl₃): δ=1.29 (t, 3H), 1.56 (s, 2H), 2.33 (s, 3H), 2.60-2.94 (m, 4H), 3.75 (s, 3H), 3.87 (d, 2H), 4.27 (q, 2H), 4.52 (d, 1H), 4.69 (s, 2H), 6.51-6.59 (m, 1H), 6.67-6.81 (m, 2H), 7.00 (d, 1H), 7.16-7.35 (m, 5H), 7.62-7.71 (m, 2H)

### Example 18

### Ethyl(2-methyl-4-{[4-methyl-7-[4-(trifluoromethyl)phenyl]-3,4-dihydro-2(1H)-isoquinolinyl]sulfonyl}phenoxy)acetate

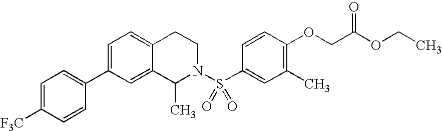

A suspension of 200 mg (0.363 mmol) of ethyl(2-methyl-{[7-{[(trifluoromethyl)sulfonyl]oxy}-4-methyl-3,4-dihydro-2(1H)-isoquinolinyl]sulfonyl}phenoxy)acetate, 86.1 mg (0.453 mmol) of 4-(trifluoromethyl)phenylboronic acid, 564 mg (1.813 mmol) of potassium phosphate trihydrate and 8.4 mg (0.007 mmol) of tetrakis(triphenylphosphine)palladium in 2 ml of toluene is heated to reflux under argon for 2 hours. After cooling to room temperature, the mixture is stirred for another 2 days. The mixture is then admixed with ethyl acetate and water, the aqueous phase is extracted twice with ethyl acetate, and the combined organic phases are dried over sodium sulfate and concentrated. After purification by HPLC, 92 mg (46% of theory) of the desired product are obtained.

HPLC (method 1): Rₜ=5.37 min.

MS (ESIpos): 548 (M+H)⁺

¹H NMR (300 MHz, CDCl₃): δ=1.26 (t, 3H), 1.52 (d, 3H), 2.23 (s, 3H), 2.62-2.85 (m, 2H), 3.45 (ddd, 1H), 3.89 (dddd, 1H), 4.25 (q, 2H), 4.64 (s, 2H), 5.20 (q, 1H), 6.66 (d, 1H), 7.09 (d, 1H), 7.27 (dd, 1H), 7.35 (dd, 1H), 78.57 (m, 2H), 7.62 (d, 2H), 7.68 (s, 1H), 7.70 (d, 1H).

### Example 19

### Ethyl(2-methyl-4-{[1,4,4-trimethyl-7-(4-fluorophenyl)-3,4-dihydro-2(1H)-isoquinolinyl]sulfonyl}phenoxy)acetate

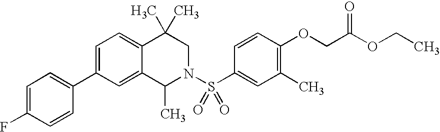

A suspension of 220 mg (0.380 mmol) of ethyl(2-methyl-{[7-{[(trifluoromethyl)sulfonyl]oxy}-1,4,4-trimethyl-3,4-dihydro-2(1H)-isoquinolinyl]sulfonyl}phenoxy)acetate, 66.4 mg (0.474 mmol) of 4-fluorophenylboronic acid, 590 mg (1.898 mmol) of potassium phosphate trihydrate and 8.8 mg (0.008 mmol) of tetrakis(triphenylphosphine)palladium in 5 ml of toluene is heated to reflux under argon for 2 hours. After cooling, another 13.2 mg (0.094 mmol) of 4-fluorophenylboronic acid and 1.8 mg (0.0016 mmol) of tetrakis(triphenylphosphine)palladium are added and the mixture is heated to reflux for 3 hours. After cooling to room temperature, the mixture is admixed with ethyl acetate and water, the aqueous phase is extracted twice with ethyl acetate, and the combined organic phases are dried over sodium sulfate and concentrated. After purification by HPLC, 114 mg (57% of theory) of the desired product are obtained.

HPLC (method 1): Rₜ=5.65 min.

MS (ESIpos): 526 (M+H)⁺

¹H NMR (300 MHz, CDCl₃): δ=1.23-1.37 (m, 12H), 2.33 (s, 3H), 3.18 (d, 1H), 3.54 (d, 1H), 4.27 (q, 2H), 4.69 (s, 2H), 5.16 (q, 1H), 6.75 (d, 1H), 7.08-7.16 (m, 3H), 7.35 (d, 1H), 7.35 (s, 1H), 7.46-7.51 (m, 2H), 7.68-7.71 (m, 2H).

### Example 20

### Ethyl(2-methyl-4-{[1,4,4-trimethyl-7-(4-methoxyphenyl)-3,4-dihydro-2(1H)isoquinolinyl]sulfonyl}phenoxy)acetate

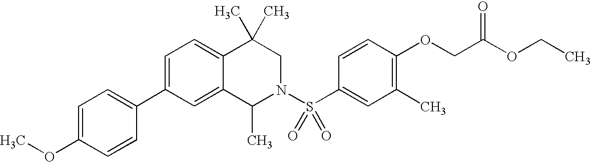

Analogously to the preparation of ethyl(2-methyl-4-{[1,4,4-trimethyl-7-(4-fluorophenyl)-3,4-dihydro-2(1H)-isoquinolinyl]sulfonyl}phenoxy)acetate 220 mg (0.380 mmol) of ethyl(2-methyl-{[7-{[(trifluoromethyl)sulfonyl]oxy}-1,4,4-trimethyl-3,4-dihydro-2(1H)-isoquinolinyl]sulfonyl}phenoxy)acetate, 86.5 mg (0.514 mmol) of 4-methoxyphenylboronic acid, 590 mg (1.898 mmol) of potassium phosphate trihydrate and 10.68 mg (0.00816 mmol) of tetrakis(triphenylphosphine)palladium in 5 ml of toluene are used to obtain, after purification by HPLC, 149 mg (73% of theory) of the desired product.

HPLC (method 1): Rₜ=5.58 min.

MS (ESIpos): 538 (M+H)⁺

¹H NMR (300 MHz, CDCl₃): δ=1.23-1.37 (m, 12H), 2.32 (s, 3H), 3.18 (d, 1H), 3.53 (d, 1H), 3.85 (s, 3H), 4.27 (q, 2H), 4.69 (s, 2H), 5.16 (q, 1H), 6.74 (d, 8H), 6.95 (d, 2H), 7.17 (d, 1H), 7.30-7.39 (m, 2H), 7.47 (d, 2H), 7.69 (m, 2H).

### Example 21

### Ethyl(2-methyl-4-{[1,4,4-trimethyl-7-[4-(trifluoromethyl)phenyl]-3,4-dihydro-2(1H)-isoquinolinyl]sulfonyl}phenoxy)acetate

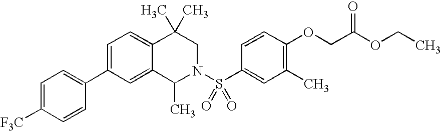

Analogously to the preparation of ethyl(2-methyl-4-{[1,4,4-trimethyl-7-(4-fluorophenyl)-3,4-dihydro-2(1H)-isoquinolinyl]sulfonyl}phenoxy)acetate, 220 mg (0.380 mmol) of ethyl(2-methyl-{[7-{[(trifluoromethyl)sulfonyl]oxy}-1,4,4-trimethyl-3,4-dihydro-2(1H)-isoquinolinyl]sulfonyl}phenoxy)acetate, 108.1 mg (0.514 mmol) of 4-(trifluoromethyl)phenylboronic acid, 590 mg (1.898 mmol) of potassium phosphate trihydrate and 10.6 mg (0.00816 mmol) of tetrakis(triphenylphosphine)palladium in 5 ml of toluene are used to obtain, after purification by HPLC, 171 mg (78% of theory) of the desired product.

HPLC (method 1): Rₜ=5.61 min.

MS (ESIpos): 576 (M+H)⁺

¹H NMR (300 MHz, CDCl₃): δ=1.23-1.38 (m, 12H), 2.33 (s, 3H), 3.18 (d, 1H), 3.56 (d, 1H), 4.27 (q, 2H), 4.69 (s, 2H), 5.18 (q, 1H), 6.75 (d, 1H), 7.22 (d, 1H), 7.37-7.44 (m, 2H), 7.61-7.71 (m, 6H).

### Example 22

### Ethyl(2-methyl-4-{[4-spirocyclobutyl-7-[4-(trifluoromethyl)phenyl]-3,4-dihydro-2(1H)-isoquinolinyl]sulfonyl}phenoxy)acetate

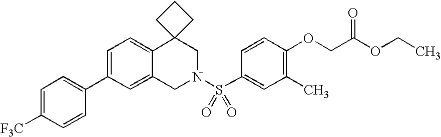

41.0 mg (0.22 mmol) of 4-trifluoromethylphenylboronic acid and 23.0 mg (0.17 mmol) of potassium carbonate are added to 0.10 g (0.17 mmol) of ethyl (2methyl-{[4-spirocyclobutyl-7-{[(trifluoromethyl)sulfonyl]oxy}-3,4-dihydro-2(1H)isoquinolinyl]sulfonyl}phenoxy)acetate in 5 ml of toluene. The mixture is blanketed with argon for 15 minutes and a spatula-tip of tetrakis(triphenylphosphine)palladium(0) is subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 30 mg (30% of theory) of the desired product are obtained.

HPLC (method 0.1): Rₜ=5.97 min.

MS (ESIpos): m/z=574 (M+H)⁺

¹H NMR (200 MHz, CDCl₃): δ=1.30 (t, 3H), 1.99-2.44 (m, 6H), 2.37 (s, 3H), 3.35 (s, 2H), 4.22 (s, 2H), 4.26 (q, 2H), 4.71 (s, 2H), 6.80 (d, 2H), 7.22 (m, 2H), 7.50 (dd, 1H), 7.60-7.76 (m, 6H).

### Example 23

### Ethyl(2-methyl-4-{[4-spirocyclobutyl-7-[4-(trifluoromethoxy)phenyl]-3,4-dihydro-2(1H)-isoquinolinyl]sulfonyl}phenoxy)acetate

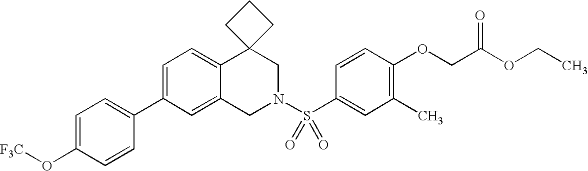

71.3 mg (0.35 mmol) of 4-trifluoromethoxyphenylboronic acid and 35.9 mg (0.26 mmol) of potassium carbonate are added to 0.10 g (0.17 mmol) of ethyl(2-methyl-{[4-spirocyclobutyl-7-{[(trifluoromethyl)sulfonyl]oxy}-3,4-dihydro-2(1H)isoquinolinyl]sulfonyl}phenoxy)acetate in 5 ml of toluene. The mixture is blanketed with argon for 15 minutes and 10 mg (0.01 mmol) of tetrakis(triphenylphosphine)palladium(0) are subsequently added. The mixture is boiled to reflux for 2 days. The mixture is cooled and freed of solvent under reduced pressure. The mixture is purified using silica gel 60 (eluent: 5:1 cyclohexane/ethyl acetate). The clean fractions are combined and freed of solvent under reduced pressure. 37 mg (36% of theory) of the desired product are obtained.

HPLC (method 1): Rₜ=6.17 min.

MS (ESIpos): m/z 590 (M+H)⁺

¹H NMR (300 MHz, CDCl₃): δ=1.29 (t, 3H), 2.04-2.27 (m, 4H), 2.30-2.47 (m, 2H), 2.36 (s, 3H), 3.35 (s, 2H), 4.21-4.32 (m, 4H), 4.70 (s, 2H), 6.79 (d, 1H), 7.17 (d, 1H), 7.26-7.29 (m, 2H), 7.45 (dd, 1H) 7.53 (d, 2H) 7.64 (d, 1H), 7.67 (s, 1H), 7.69 (m, 1H).

### Example 24

### Ethyl(2-methyl-4-{[7-[3-fluorophenyl]-4-spirocyclopentyl-3,4-dihydro-2(1H)isoquinolinyl]sulfonyl}phenoxy)acetate

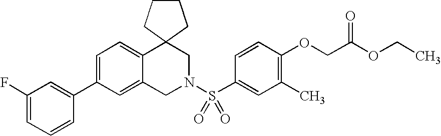

70.95 mg (0.51 mmol) of 3-fluorophenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfony]]oxy}-3,4-dihydro-2(1H)-isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 121 mg (89% of theory) of the desired product are obtained.

HPLC (method 1): Rₜ=5.89 min.

MS (ESIpos): m/z=538 (M+H)⁺

¹H NMR (200 MHz, CDCl₃): δ=1.30 (t, 3H), 1.75-2.02 (m, 8H), 2.36 (s, 3H), 3.03 (s, 2H), 4.20-4.37 (m, 4H), 4.71 (s, 2H), 6.75-6.85, (m, 1H), 6.94-7.09 (m, 1H), 7.15-7.22 (m, 2H), 7.30-7.48 (m, 4H) 7.61-7.74 (m, 2H).

### Example 25

### Ethyl(2-methyl-4-{[7-[4-methoxyphenyl]-4-spirocyclopentyl-3,4-dihydro-2(1H)isoquinolinyl]sulfonyl}phenoxy)acetate

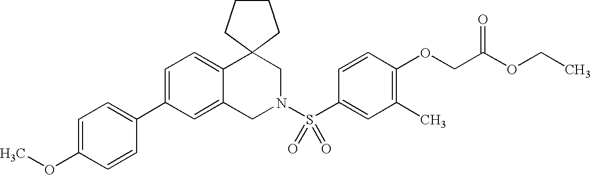

77.05 mg (0.51 mmol) of 4-methoxyphenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfonyl]oxy}-3,4-dihydro-2(1H)-isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 84 mg (60% of theory) of the desired product are obtained.

HPLC (method 1): Rₜ=5.81 min.

MS (ESIpos): m/z=550 (M+H)⁺

¹H NMR (200 MHz, CDCl₃): δ=1.29 (t, 3H), 1.73-2.00 (m, 8H), 2.36 (s, 3H), 3.02 (s, 2H), 3.84 (s, 3H), 4.18-4.37 (m, 4H), 4.71 (s, 2H), 6.72-6.84 (m, 1H), 6.90-7.00 (m, 2H), 7.17 (s, 1H), 7.27-7.51 (m, 4H), 7.62-7.74 (m, 2H).

### Example 26

### Ethyl(2-methyl-4-{[7-[3-methoxyphenyl]-4-spirocyclopentyl-3,4-dihydro-2(1H)isoquinolinyl]sulfonyl}phenoxy)acetate

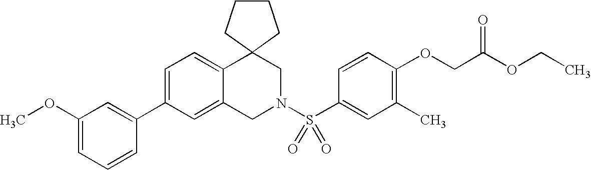

77.05 mg (0.51 mmol) of 3-methoxyphenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfonyl]oxy}-3,4-dihydro-2(1H)-isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 98 mg (70% of theory) of the desired product are obtained.

HPLC (method 1): Rₜ=5.91 min.

MS (ESIpos): m/z 550 (M+H)⁺

¹H NMR (300 MHz, CDCl₃): δ=1.29 (t, 3H), 1.79-1.96 (m, 8H), 2.36 (s, 3H), 3.04 (s, 2H), 3.85 (s, 3H), 4.22-4.31 (m, 4H), 4.70 (s, 2H), 6.76-6.82 (m, 1H), 6.87 (dd, 1H), 7.02-7.07 (m, 1H), 7.08-7.13 (m, 1H), 7.18-7.22 (m, 1H), 7.28-7.36 (m, 2H), 7.38-7.44 (m, 1H), 7.62-7.70 (m, 2H).

### Example 27

### Ethyl(2-methyl-4-{[4-spirocyclopentyl-7-[3-(trifluoromethyl)phenyl]-3,4-dihydro-2(1H)-isoquinolinyl]sulfonyl}phenoxy)acetate

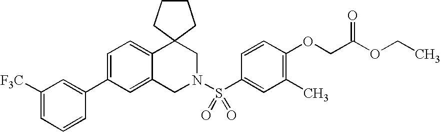

96.31 mg (0.51 mmol) of 3-trifluoromethylphenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfonyl]oxy}-3,4-dihydro-2(1H)isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux for 6 hours. The mixture is cooled and freed of solvent under reduced pressure. The desired product is obtained in a mixture with the reactant; the purification is effected at the stage of the carboxylic acid (see Example 44).

LC-MS (method 3): Rₜ=3.40 min.

MS (ESIpos): m/z=588 (M+H)⁺.

### Example 28

### Ethyl(2-methyl-4-{[7-[4-methylphenyl]-4-spirocyclopentyl-3,4-dihydro-2(1H)isoquinolinyl]sulfonyl}phenoxy)acetate

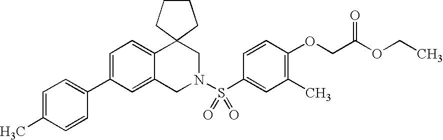

68.94 mg (0.51 mmol) of 4-methylphenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfonyl]oxy}-3,4-dihydro-2(1H)-isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 116 mg (86% of theory) of the desired product are obtained.

HPLC (method 1): Rₜ=6.22 min.

MS (ESIpos): m/z 534 (M+H)⁺

¹H NMR (300 MHz, CDCl₃): δ=1.29 (t, 3H), 1.79-1.97 (m, 8H), 2.36 (d, 6H), 3.03 (s, 2H), 4.22-4.32 (m, 4H), 4.70 (s, 2H), 6.79 (d, 1H), 7.17-7.21 (m, 2H), 7.23 (s, 2H), 7.31 (d, 1H), 7.39-7.60 (m, 2H), 7.63-7.66 (m, 2H).

### Example 29

### Ethyl(2-methyl-4-{[7-[3-methylphenyl]-4-spirocyclopentyl-3,4-dihydro-2(1H)isoquinolinyl]sulfonyl}phenoxy)acetate

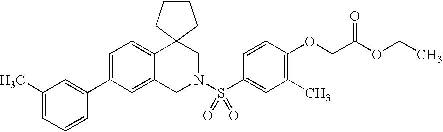

68.94 mg (0.51 mmol) of 3-methylphenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfonyl]oxy}-3,4-dihydro-2(1H)-isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 126 mg (93% of theory) of the desired product are obtained.

HPLC (method 1): Rₜ=6.10 min.

MS (ESIpos): m/z=534 (M+H)⁺

¹H NMR (200 MHz, CDCl₃): δ=1.29 (t, 3H), 1.80-1.97 (m, 8H), 2.38 (d, 6H), 3.03 (s, 2H), 4.20-4.33 (m, 4H), 4.71 (s, 2H), 6.79 (d, 1H), 7.11-7.18 (m, 1H), 7.18-7.22 (m, 1H), 7.29-7.36 (m, 4H), 7.38-7.46 (m, 1H), 7.62-7.71 (m, 2H).

### Example 30

### Ethyl(2-methyl-4-{[4-spirocyclopentyl-7-[4-(trifluoromethoxy)phenyl]-3,4-dihydro-2(1H)-isoquinolinyl]sulfonyl}phenoxy)acetate

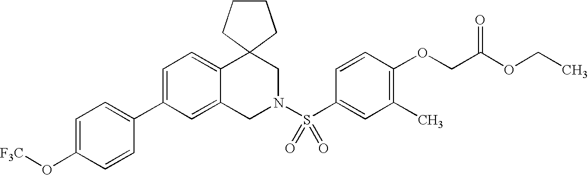

104.4 mg (0.51 mmol) of 4-trifluoromethoxyphenylboronic acid and 52.6 mg (0.38 mmol) of potassium carbonate are added to 0.15 g (0.25 mmol) of ethyl(2methyl-{[4-spirocyclopentyl-7-{[(trifluoromethyl)sulfonyl]oxy}-3,4-dihydro-2(1H)isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 133 mg (87% of theory) of the desired product are obtained.

HPLC (method 1): Rₜ=6.30 min.

MS (ESIpos): m/z=604 (M+H)⁺

¹H NMR (300 MHz, CDCl₃): δ=1.29 (t, 3H), 1.56-1.80 (m, 8H), 2.36 (s, 3H), 3.04 (s, 2H), 4.26 (s, 2H), 4.28 (q, 2H), 4.69 (s, 2H), 6.79 (d, 1H), 7.18 (d, 1H), 7.24 (d, 2H), 7.33 (d, 1H), 7.39 (dd, 1H), 7.52 (d, 2H), 7.55 (s, 1H), 7.57 (d, 1H).

### Example 31

### Ethyl(2-methyl-4-{[4-spirocyclopentyl-7-[3-(trifluoromethoxy)phenyl]-3,4-dihydro-2(1H)-isoquinolinyl]sulfonyl})phenoxy)acetate

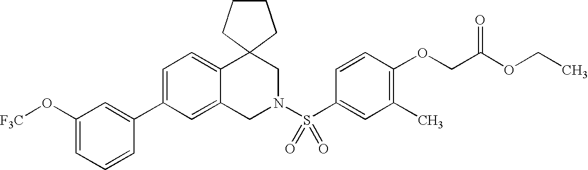

104.42 mg (0.51 mmol) of 3-trifluoromethoxyphenylboronic acid and 52.56 mg (0.38 mmol) of potassium carbonate are added to 0.15 g (0.25 mmol) of ethyl(2-methyl-{[4-spirocyclopentyl-7-{[(trifluoromethyl)sulfonyl]oxy}-3,4-dihydro-2(1H)isoquinolinyl]sulfonyl}phenoxy)acetate in 5 ml of toluene. The mixture is blanketed with argon for 15 minutes and 13.4 mg (0.01 mmol) of tetrakis(triphenylphosphine)palladium(0) are subsequently added. The mixture is boiled to reflux overnight. The mixture is cooled and freed of solvent under reduced pressure. The mixture is purified using silica gel 60 (eluent: 5:1 cyclohexane/ethyl acetate). The clean fractions are combined and freed of solvent under reduced pressure. 143 mg (93% of theory) of the desired product are obtained.

HPLC (method 1): Rₜ=6.27 min.

MS (ESIpos): m/z 604 (M+H)⁺

¹H NMR (300 MHz, CDCl₃): δ=1.29 (t, 3H), 1.80-1.88 (m, 8H), 2.36 (s, 3H), 3.04 (s, 2H), 4.21-4.32 (m, 4H), 4.70 (s, 2H), 6.79 (d, 1H), 7.14-7.20 (m, 2H), 7.31-7.46 (m, 5H), 7.63-7.69 (m, 2H).

### Example 32

### Ethyl(2-methyl-4-{[4-(2-phenylethyl)-7-[4-(trifluoromethyl)phenyl]-3,4-dihydro-2(1H)-isoquinolinyl]sulfonyl}phenoxy)acetate

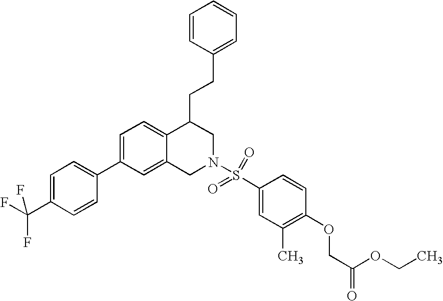

Under argon supply, 0.209 g (0.33 mmol) of ethyl(2-methyl-4-{[4-(2-phenylethyl)-7-{[(trifluoromethyl)sulfonyl]oxy}-3,4-dihydro-2(1H)-isoquinolinyl]sulfonyl}-phenoxy)acetate and 77.3 mg (0.41 mmol) of 4-trifluoromethylphenylboronic acid are dissolved in 1.74 ml of dioxane. The flask is evacuated three times and filled with argon each time. The mixture is admixed with 7.53 mg (0.01 mmol) of tetrakis(triphenylphosphine)palladium(0) and 506.8 mg (1.63 mmol) of potassium phosphate trihydrate. The evacuation operation is repeated twice more. The mixture is heated to reflux for 2 hours and the mixture is left to stand overnight. For the workup, the supernatant is decanted from the phosphate and concentrated under reduced pressure. The residue is dissolved in dichloromethane and purified using silica gel 60 (eluent: dichloromethane). The clean fractions are combined and freed of solvent under reduced pressure. 159 mg (76% of theory) of the desired product are obtained.

HPLC (method 1): Rₜ=6.43 min.

MS (ESIpos): m/z 638 (M+H)⁺

¹H NMR (300 MHz, CDCl₃): δ=1.29 (t, 3H), 2.04 (s, 1H), 2.08-2.22 (m, 1H), 2.34 (s, 3H), 2.65-2.79 (m, 1H), 2.81-2.95 (m, 3H), 3.81-3.89 (m, 1H), 3.98 (d, 1H), 4.27 (q, 2H), 4.61 (s, 1H), 4.64-4.71 (s, 2H), 6.77 (d, 2H), 7.17-7.34 (m, 6H), 7.35-7.41 (m, 1H), 7.57-7.72 (m, 6H).

### Example 33

### (2-Methyl-4-{[4-methyl-7-[4-(trifluoromethyl)phenyl]-3,4-dihydro-2(1H)-isoquinolinyl]sulfonyl}phenoxy)acetic acid

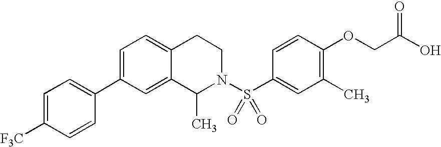

A solution of 75.0 mg (0.137 mmol) of ethyl(2-methyl-4-{[4-methyl-7-(4-fluorophenyl)-3,4-dihydro-2(1H)-isoquinolinyl]sulfonyl}phenoxy)acetate in 1 ml of ethanol is admixed with 1 ml (2.0 mmol) of 2 M sodium hydroxide solution and stirred at room temperature overnight. Subsequently, the mixture is concentrated under reduced pressure, acidified with 1 N hydrochloric acid and stirred overnight. The resultant precipitate is filtered off and dried under reduced pressure. 58 mg (79% of theory) of the desired product are obtained as a colorless solid.

HPLC (method 1): Rₜ=5.22 min.

MS (ESIpos): 520 (M+H)⁺

¹H NMR (300 MHz, DMSO-d₆): δ=1.42 (d, 3H), 2.12 (s, 3H), 2.60-2.78 (m, 2H), 3.43 (ddd, 1H), 3.79 (ddd, 1H), 4.75 (s, 2H), 5.16 (q, 1H), 6.92 (d, 1H), 7.13 (d, 1H), 7.46 (dd, 114); 7.59 (d, 3H), 7.79 (d, 1H), 7.80 (s, 1H), 7.86 (s, 1H), 7.88 (d, 1H), 13.08 (broad, s, 1H).

### Example 34

### (2-Methyl-4-{[1,4,4-trimethyl-7-(4-fluorophenyl)-3,4-dihydro-2(1H)-isoquinolinyl]-sulfonyl}phenoxy)acetic acid

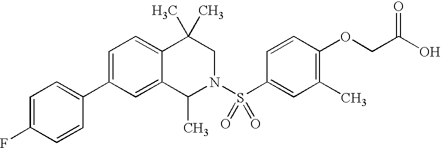

A solution of 80.0 mg (0.152 mmol) of ethyl(2-methyl-4-{[1,4,4-trimethyl-7-(4-fluorophenyl)-3,4-dihydro-2(1H)-isoquinolinyl]sulfonyl}phenoxy)acetate in 2 ml of ethanol is admixed with 1 ml (2.0 mmol) of 2 M sodium hydroxide solution and stirred at room temperature overnight. Subsequently, the mixture is concentrated under reduced pressure, acidified with 1 N hydrochloric acid and stirred for 1 hour. The resultant precipitate is filtered off and dried under reduced pressure. 60 mg (79% of theory) of the desired product are obtained as a colorless solid.

HPLC (method 1): Rₜ=5.17 min.

MS (ESIpos): 498 (M+H)⁺

¹H NMR (300 MHz, DMSO-d₆): δ=1.14 (s, 3H), 1.19 (d, 3H), 1.31 (s, 3H), 2.28 (s, 3H), 3.13 (d, 1H), 3.57 (d, 1H), 4.82 (s, 2H), 5.09 (q, 1H), 7.02 (d, 1H), 7.27 (t, 2H), 7.43 (d, 2H), 7.45 (s, 1H), 7.65-7.72 (m, 4H), 13.20 (broad, s, 1H).

### Example 35

### (2-Methyl-4-{[1,4,4-trimethyl-7-(4-methoxyphenyl)-3,4-dihydro-2(1H)-isoquinolinyl]sulfonyl}phenoxy)acetic acid

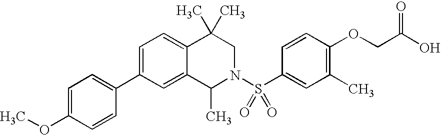

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 121.0 mg (0.225 mmol) of ethyl(2-methyl-4-{[1,4,4-trimethyl-7-(4-methoxyphenyl)-3,4-dihydro-2(1H)-isoquinolinyl]sulfonyl}phenoxy)acetate and 1 ml (2.0 mmol) of 2 M sodium hydroxide solution in 2 ml of ethanol are used to obtain 103 mg (90% of theory) of the desired product.

HPLC (method 1): Rₜ=5.10 min.

MS (ESIpos): 510 (M+H)⁺

¹H NMR (300 MHz, DMSO-d₆): δ=1.13 (s, 3H), 1.19 (d, 3H), 1.30 (s, 3H), 2.26 (s, 3H), 3.13 (d, 1H), 3.56 (d, 1H), 4.82 (s, 2H), 5.08 (q, 1H), 7.00 (d, 2H), 7.02 (d, 1H), 7.38 (s, 1H), 7.42 (s, 2H), 7.58 (d, 2H), 7.63-7.70 (m, 2H), 13.21 (broad, s, 1H).

### Example 36

### (2-Methyl-4-{[1,4,4-trimethyl-7-[4-(trifluoromethyl)phenyl]-3,4-dihydro-2(1H)isoquinolinyl]sulfonyl}phenoxy)acetic acid

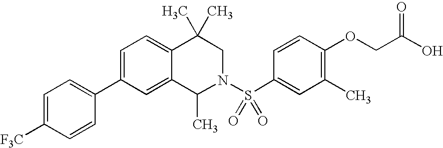

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 141.0 mg (0.245 mmol) of ethyl(2-methyl-4-{[1,4,4-trimethyl-7-[4-(trifluoromethyl)phenyl]-3,4-dihydro-2(1H)-isoquinolinyl]sulfonyl}phenoxy)acetate and 1.5 ml (3.0 mmol) of 2 M sodium hydroxide solution in 2 ml of ethanol are used to obtain 126 mg (94% of theory) of the desired product.

HPLC (method 1): Rₜ=5.38 min.

MS (ESIpos): 548 (M+H)⁺

¹H NMR (300 MHz, DMSO-d₆): δ=1.15 (s, 3H), 1.20 (d, 3H), 1.32 (s, 3H), 2.26 (s, 3H), 3.15 (d, 1H), 3.58 (d, 1H), 4.79 (s, 2H), 5.12 (q, 1H), 7.01 (d, 1H), 7.49 (d, 1H), 7.55 (s, 2H), 7.67 (d, 1H), 7.69 (s, 1H), 7.79 (d, 2H), 7.89 (d, 2H), 13.28 (broad, s, 1H).

### Example 37

### (2-Methyl-4-{[4-spirocyclobutyl-7-[4-(trifluoromethyl)phenyl]-3,4-dihydro-2(1H)isoquinolinyl]sulfonyl}phenoxy)acetic acid

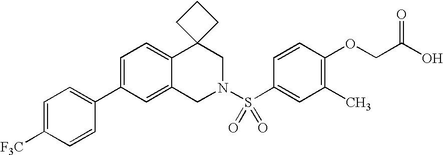

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 30.0 mg (0.052 mmol) of ethyl(2-methyl-4-{[4-spirocyclobutyl-7-[4-(trifluoromethyl)phenyl]-3,4-dihydro-2(1H)-isoquinolinyl]sulfonyl}phenoxy)acetate and 0.12 ml (0.12 mmol) of 1 M sodium hydroxide solution in 8 ml of ethanol are used to obtain, after purification using silica gel (1:1 cyclohexane/ethyl acetate), 10 mg (35% of theory) of the desired product.

HPLC (method 1): Rₜ=5.40 min.

MS (ESIpos): 548 (M+H)⁺

¹H NMR (300 MHz, DMSO-d₆): δ=1.15 (s, 3H), 1.20 (d, 3H), 1.32 (s, 3H), 2.26 (s, 3H), 3.15 (d, 1H), 3.58 (d, 1H), 4.79 (s, 2H), 5.12 (q, 1H), 7.01 (d, 1H), 7.49 (d, 1H), 7.55 (s, 2H), 7.67 (d, 1H), 7.69 (s, 1H), 7.79 (d, 2H), 7.89 (d, 2H), 13.28 (broad, s, 1H).

### Example 38

### (2-Methyl-4-{[4-spirocyclopentyl-7-phenyl]-3,4-dihydro-2(1H)-isoquinolinyl]-sulfonyl}phenoxy)acetic acid

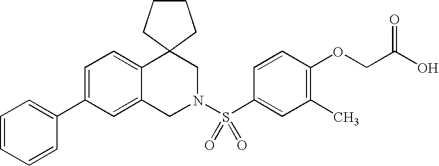

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 41.0 mg (0.079 mmol) of ethyl(2-methyl-4-{[4-spirocyclopentyl-7-phenyl]-3,4-dihydro-2(1H)-isoquinolinyl]sulfonyl}phenoxy)acetate and 0.16 ml (0.16 mmol) of 1 M sodium hydroxide solution in 5 ml of ethanol are used to obtain, after purification using silica gel (1:1 cyclohexane/ethyl acetate→10:1 methylene chloride/methanol), 30 mg (77% of theory) of the desired product.

LC-MS (method 3): Rₜ=3.11 min.

MS (ESIpos): 492 (M+H)⁺

¹H NMR (400 MHz, DMSO-d₆): δ=1.70-1.95 (m, 8H), 2.29 (s, 3H), 2.97 (s, 2H), 4.20 (s, 2H), 4.79 (s, 2H), 7.07 (d, 1H), 7.33 (t, 1H), 7.37-7.47 (m, 4H), 7.51 (dd, 1H), 7.60-7.69 (m, 5H).

### Example 39

### (2-Methyl-4-{[7-[4-fluorophenyl]-4-spirocyclopentyl-3,4-dihydro-2(1H)-isoquinolinyl]sulfonyl}phenoxy)acetic acid

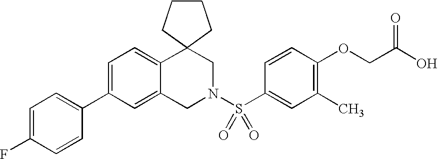

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 67.0 mg (0.083 mmol) of ethyl(2-methyl-4-{[4-spirocyclopentyl-7-(4-fluoro)phenyl-3,4-dihydro-2(1H)-isoquinolinyl]sulfonyl}phenoxy)acetate and 0.20 ml (0.20 mol) of 1 M sodium hydroxide solution in 5 ml of ethanol are used to obtain, after purification using silica gel (1:1 cyclohexane/ethyl acetate→10:1′ methylene chloride/methanol) and by HPLC, 7 mg (16% of theory) of the desired product.

HPLC (method 1): Rₜ=5.38 min.

MS (ESIpos): 510 (M+H)⁺

¹H NMR (300 MHz, DMSO-d₆): δ=1.68-1.95 (m, 8H), 2.29 (s, 3H), 2.98 (s, 2H), 4.20 (s, 2H), 4.82 (s, 2H), 7.08 (d, 1H), 7.22-7.34 (m, 3H), 7.40 (d, 1H), 7.41 (s, 1H), 7.49 (dd, 1H), 7.54 (d, 1H), 7.62-7.70 (m, 3H), 13.13 (broad, s, 1H).

### Example 40

### (2-Methyl-4-{[7-[3-fluorophenyl]-4-spirocyclopentyl-3,4-dihydro-2(1H)-isoquinolinyl]sulfonyl-3 phenoxy)acetic acid

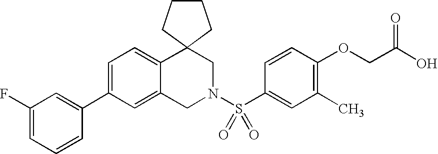

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 93.0 mg (0.173 mmol) of ethyl(2-methyl-4-{[4-spirocyclopentyl-7-(3-fluoro)phenyl-3,4-dihydro-2(1H)-isoquinolinyl]sulfonyl}phenoxy)acetate and 0.259 ml (0.259 mmol) of 1 M sodium hydroxide solution in 5 ml of ethanol are used to obtain, after extraction of the aqueous phase with ethyl acetate and removal of the solvent under reduced pressure, 88 mg (99% of theory) of the desired product.

HPLC (method 1): Rₜ=5.49 min.

MS (ESIpos): 510 (M+H)⁺

¹H NMR (200 MHz, DMSO-d₆): δ=1.70-1.91 (m, 8H), 2.29 (s, 3H), 2.96 (s, 2H), 4.17 (s, 2H), 4.84 (s, 2H), 7.00 (d, 2H), 7.07 (d, 1H), 7.36 (d, 1H), 7.38 (s, 1H), 7.46 (d, 1H), 7.57 (d, 2H), 7.66 (d, 1H), 7.68 (s, 1H), 13.19 (broad s, 1H).

### Example 41

### (2-Methyl-4-{[7-[4-methoxyphenyl]-4-spirocyclopentyl-3,4-dihydro-2(1H)isoquinolinyl]sulfonyl}phenoxy)acetic acid

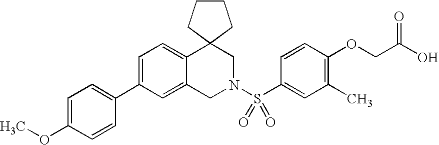

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 53.0 mg (0.096 mmol) of ethyl(2-methyl-4-[4-spirocyclopentyl-7-(4-methoxy)phenyl-3,4-dihydro-2(1H)-isoquinolinyl]sulfonyl}phenoxy)acetate and 0.145 ml (0.145 mmol) of 1 M sodium hydroxide solution in 5 ml of ethanol are used to obtain, after extraction of the aqueous phase with ethyl acetate and removal of the solvent under reduced pressure, 47 mg (93% of theory) of the desired product.

HPLC (method 1): Rₜ=5.41 min.

MS (ESIpos): 522 (M+H)⁺

¹H NMR (200 MHz, DMSO-d₆): δ=1.72-1.93 (m, 8H), 2.28 (s, 3H), 2.97 (s, 2H), 3.34 (s, 3H), 4.19 (s, 2H), 4.80 (s, 2H), 7.05 (d, 1H), 7.17 (m, 1H), 7.36-7.60 (m, 6H), 7.65 (d, 1H), 7.67 (s, 1H), 13.35 (broad, s, 1H).

### Example 42

### (2-Methyl-4-{[7-[3-methoxyphenyl]-4-spirocyclopentyl-3,4-dihydro-2(1H)-isoquinolinyl]sulfonyl}phenoxy)acetic acid

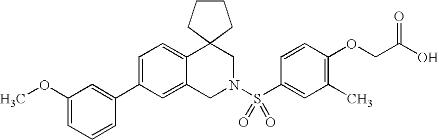

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 68.0 mg (0.124 mmol) of ethyl(2-methyl-4-{[4-spirocyclopentyl-7-(3-methoxy)phenyl-3,4-dihydro-2(1H)-isoquinolinyl]sulfonyl}phenoxy)acetate and 0.186 ml (0.186 mmol) of 1 M sodium hydroxide solution in 5 ml of ethanol are used to obtain, after extraction of the aqueous phase with ethyl acetate and removal of the solvent under reduced pressure, 61 mg (95% of theory) of the desired product.

HPLC (method 1): Rₜ=5.42 min.

MS (ESIpos): 522 (M+H)⁺

¹H NMR (300 MHz, DMSO-d₆): δ=1.72-1.91 (m, 8H), 2.29 (s, 3H), 2.98 (s, 2H), 3.81 (s, 3H), 4.20 (s, 2H), 4.81 (s, 2H), 6.90 (dd, 1H), 7.07 (d, 1H), 7.17 (dd, 1H), 7.18 (dd, 1H), 7.33 (d, 1H), 7.39 (d, 1H), 7.44 (d, 1H), 7.51 (dd, 1H), 7.66 (dd, 1H), 7.67 (s, 1H).

### Example 43

### (2-Methyl-4-{[4-spirocyclopentyl-7-[4-(trifluoromethyl)phenyl]-3,4-dihydro-2(1H)isoquinolinyl]sulfonyl}phenoxy)acetic acid

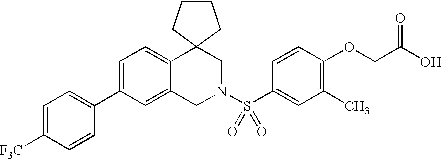

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 200.0 mg (0.34 mmol) of ethyl(2-methyl-4-{[4-spirocyclopentyl-7-(4-trifluoromethyl)phenyl-3,4-dihydro-2(1H)-isoquinolinyl]sulfonyl}phenoxy)acetate and 0.70 ml (0.70 mmol) of 1 M sodium hydroxide solution in 10 ml of ethanol are used to obtain, after extraction of the aqueous phase with ethyl acetate, removal of the solvent under reduced pressure and purification using silica gel (20.1 methylene chloride/methanol), 133 mg (70% of theory) of the desired product.

HPLC (method 1): Rₜ=5.60 min.

MS (ESIpos): 560 (M+H)⁺

¹H NMR (300 MHz, DMSO-d₆): δ=1.72-1.92 (m, 8H), 2.29 (s, 3H), 2.98 (s, 2H), 4.21 (s, 2H), 4.82 (s, 2H), 7.07 (d, 1H), 7.46 (d, 1H), 7.52 (d, 1H), 7.59 (dd, 1H), 7.66 (dd, 1H), 7.68 (s, 1H), 7.79 (d, 2H), 7.86 (d, 2H).

### Example 44

### (2-Methyl-4-{[4-spirocyclopentyl-7-[3-(trifluoromethyl)phenyl]-3,4-dihydro-2(1H)isoquinolinyl]sulfonyl}phenoxy)acetic acid

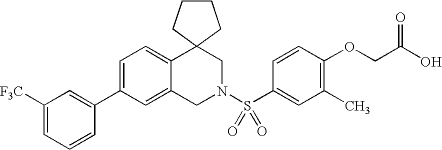

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 142.0 mg (0.242 mmol) of ethyl(2-methyl-4-{[4-spirocyclopentyl-7-(3-trifluoromethyl)phenyl-3,4-dihydro-2(1H)-isoquinolinyl]sulfonyl}phenoxy)acetate and 0.48 ml (0.48 mmol) of 1 M sodium hydroxide solution in 3 ml of ethanol are used to obtain, after extraction of the aqueous phase with ethyl acetate, removal of the solvent under reduced pressure and purification using silica gel (20:1 methylene chloride/methanol), 87 mg (64% of theory) of the desired product.

HPLC (method 1): Rₜ=5.60 min.

MS (ESIpos): 560 (M+H)⁺

¹H NMR (300 MHz, DMSO-d₆): δ=1.72-1.92 (m, 8H), 2.29 (s, 3H), 2.99 (s, 2H), 4.21 (s, 2H), 4.83 (s, 2H), 7.03-7.13 (m, 2H), 7.43 (d, 1H), 7.56 (s, 1H), 7.59 (dd, 1H), 7.63-7.72 (m, 3H), 7.95 (m, 2H).

### Example 45

### (2-Methyl-4-{[7-[4-methylphenyl]-4-spirocyclopentyl-3,4-dihydro-2(1H)-isoquinolinyl]sulfonyl}phenoxy)acetic acid

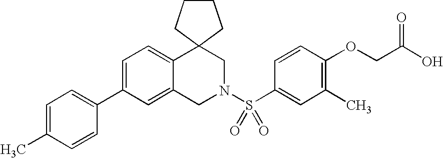

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 86.0 mg (0.161 mmol) of ethyl(2-methyl-4-{[4-spirocyclopentyl-7-(4-methyl)phenyl-3,4-dihydro-2(1H)-isoquinolinyl]sulfonyl}phenoxy)acetate and 0.24 ml (0.24 mmol) of 1 M sodium hydroxide solution in 3 ml of ethanol are used to obtain, after extraction of the aqueous phase with ethyl acetate and removal of the solvent under reduced pressure, 78 mg (96% of theory) of the desired product.

HPLC (method 1): Rₜ=5.71 min.

MS (ESIpos): 506 (M+H)⁺

¹H NMR (200 MHz, CDCl₃): δ=1.75-2.02 (m, 8H), 2.35 (s, 3H), 2.38 (s, 3H), 3.03 (s, 2H), 4.26 (s, 2H), 4.78 (s, 2H), 6.82 (d, 1H), 7.20 (s, 1H), 7.23 (d, 2H), 7.30 (d, H), 7.46 (s, 1H), 7.42 (d, 2H), 7.68 (s, 1H), 7.69 (d, 1H).

### Example 46

### (2-Methyl-4-{[7-[3-methylphenyl]4-spirocyclopentyl-3,4-dihydro-2(1H)-isoquinolinyl]sulfonyl}phenoxy)acetic acid

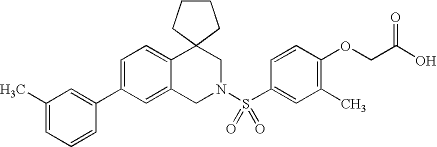

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 94.0 mg (0.176 mmol) of ethyl(2-methyl-4-{[4-spirocyclopentyl-7-(3-methyl)phenyl-3,4-dihydro-2(1H)-isoquinolinyl]sulfonyl}phenoxy)acetate and 0.26 ml (0.26 mmol) of 1 M sodium hydroxide solution in 3 ml of ethanol are used to obtain, after extraction of the aqueous phase with ethyl acetate, drying over sodium sulfate and removal of the solvent under reduced pressure, 86 mg (97% of theory) of the desired product.

HPLC (method 1): Rₜ=5.66 min.

MS (ESIpos): 506 (M+H)⁺

¹H NMR (300 MHz, DMSO-d₆): δ=1.70-1.93 (m, 10H), 2.29 (s, 3H), 2.35 (s, 3H), 2.98 (s, 2H), 4.20 (s, 2H), 4.82 (s, 2H), 7.08 (d, 1H), 7.13 (d, 1H), 7.30 (t, 1H), 7.38-7.47 (m, 4H), 7.50 (dd, 1H), 7.66 (d, 1H), 7.68 (s, 1H).

### Example 47

### (2-Methyl-4-{[4-spirocyclopentyl-7-[4-(trifluoromethoxy)phenyl]-3,4-dihydro-2(1H)-isoquinolinyl]sulfonyl}phenoxy)acetic acid

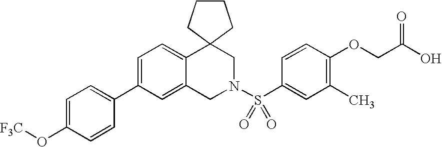

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 94.0 mg (0.156 mmol) of ethyl(2-methyl-4-{[4-spirocyclopentyl-7-(4-trifluoromethoxy)phenyl-3,4-dihydro-2(1H)-isoquinolinyl]sulfonyl}phenoxy)acetate and 0.23 ml (0.23 mmol) of 1 M sodium hydroxide solution in 3 ml of ethanol are used to obtain, after extraction of the aqueous phase with ethyl acetate, drying over sodium sulfate and removal of the solvent under reduced pressure, 86 mg (96% of theory) of the desired product.

HPLC (method 1): Rₜ=5.73 min.

MS (ESIpos): 576 (M+H)⁺

¹H NMR (200 MHz, CDCl₃): δ=1.75-2.00 (m, 8H), 2.35 (s, 0.3H), 3.03 (s, 2H), 4.27 (s, 2H), 4.77 (s, 2H), 6.81 (d, 1H), 7.16 (s, 1H), 7.23 (d, 2H), 7.35 (s, 1H), 7.38 (d, 1H), 7.52 (d, 2H), 7.66 (s, 1H), 7.68 (d, 1H).

### Example 48

### (2-Methyl-4-{[4-spirocyclopentyl-7-[3-(trifluoromethoxy)phenyl]-3,4-dihydro 2(1H)-isoquinolinyl]sulfonyl}phenoxy)acetic acid

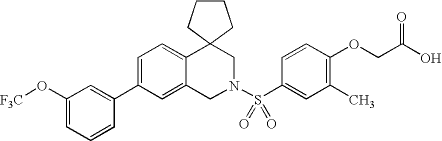

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 91.0 mg (0.151 mmol) of ethyl(2-methyl-4-{[4-spirocyclopentyl-7-(3-trifluoromethoxy)phenyl-3,4-dihydro-2(1H)-isoquinolinyl]sulfonyl}phenoxy)acetate and 0.23 ml (0.23 mmol) of 1 M sodium hydroxide solution in 3 ml of ethanol are used to obtain, after extraction of the aqueous phase with ethyl acetate, drying over sodium sulfate and removal of the solvent under reduced pressure, 85 mg (98% of theory) of the desired product.

HPLC (method 1): Rₜ=5.64 min.

MS (ESIpos): 576 (M+H)⁺

¹H NMR (200 MHz, CDCl₃): δ=1.75-2.00 (m, 8H), 2.36 (s, 3H), 3.02 (s, 2H), 4.26 (s, 2H), 4.78 (s, 2H), 6.81 (d; 1H), 7.19 (m, 2H), 7.30-7.48 (m, 5H), 7.68 (s, 1H), 7.69 (d, 1H).

### Example 49

### (4-{[7-Bromo-4-spirocyclohexyl]-3,4-dihydro-2(1H)-isoquinolinyl]sulfonyl}-2-methylphenoxy)acetic acid

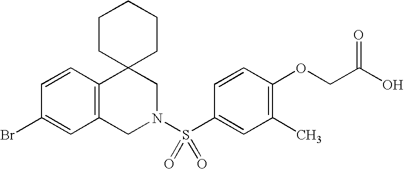

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 35.0 mg (0.065 mmol) of ethyl(2-methyl-4-{[7-bromo-4-spirocyclohexyl-3,4-dihydro-2(1H)isoquinolinyl]sulfonyl}phenoxy)acetate and 0.13 ml (0.13 mmol) of 1 M sodium hydroxide solution in 2 ml of ethanol are used to obtain, after extraction of the aqueous phase with ethyl acetate, drying over sodium sulfate and removal of the solvent under reduced pressure, 33 mg (99% of theory) of the desired product.

LC-MS (method 3): Rₜ=3.05 min.

MS (ESIpos): 508 (M+H)⁺

¹H NMR (200 MHz, CDCl₃): δ=1.45-1.90 (m, 10H), 2.36 (s, 3H), 3.22 (s, 2H), 4.15 (s, 2H) 4.78 (s, 2H), 6.81 (d, 1H), 7.14 (d, 1H), 7.25 (s, 1H), 7.31 (dd, 1H), 7.65 (s, 1H), 7.68 (d, 1H).

### Example 50

### (4-{[4-Spirocyclohexyl-7-[4-(trifluoromethyl)phenyl]-3,4-dihydro-2(1H)-isoquinolinyl]sulfonyl}phenoxy)acetic acid

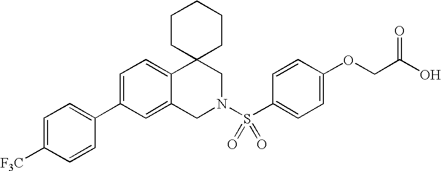

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 68.0 mg (0.116 mmol) of ethyl(4-{[4-spirocyclohexyl-7-(4-trifluoromethyl)phenyl-3,4-dihydro-2(1H)-isoquinolinyl]sulfonyl}phenoxy)acetate and 0.23 ml (0.23 mmol) of 1 M sodium hydroxide solution in 5 ml of ethanol are used to obtain, after additional addition of 0.10 ml (0.10 mmol) of 1 M sodium hydroxide solution, stirring for 2 days, extraction of the aqueous phase with ethyl acetate, removal of the solvent under reduced pressure and purification using silica gel (100:1→10:1 methylene chloride/methanol), 12 mg (15% of theory) of the desired product.

HPLC (method 1): Rₜ=5.57 min.

MS (ESIpos): 560 (M+H)⁺

¹H NMR (300 MHz, DMSO-d₆): δ=1.45-1.85 (m, 10H), 3.22 (s, 2H), 4.19 (s, 2H), 4.70 (s, 2H); 7.13 (d, 2H), 7.55 (d, 1H), 7.58 (s, 1H), 7.75-7.90 (m, 7H).

### Example 51

### (2-Methyl-4-{[7-phenyl-4-spirocyclohexyl]-3,4-dihydro-2(1H)-isoquinolinyl]-sulfonyl}phenoxy)acetic acid

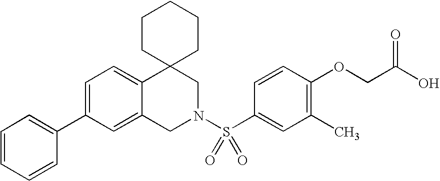

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 53.4 mg (0.100 mmol) of ethyl(2-methyl-4-{[4-spirocyclohexyl-7-phenyl-3,4-dihydro-2(1H)isoquinolinyl]sulfonyl}phenoxy)acetate and 0.20 ml (0.20 mmol) of 1 M sodium hydroxide solution in 5 ml of ethanol are used to obtain, after extraction of the aqueous phase with ethyl acetate, drying over sodium sulfate and removal of the solvent under reduced pressure and purification using silica gel (3:1 cyclohexane/ethyl acetate→10:1 methylene chloride/methanol), 30 mg (59% of theory) of the desired product.

HPLC (method 1): Rₜ=5.53 min.

MS (ESIpos): 506 (M+H)⁺

¹H NMR (300 MHz, CDCl₃): δ=1.45-1.90 (m, 10H), 2.35 (s, 3H), 3.29 (s, 2H), 4.26 (s, 2H), 4.77 (s, 2H), 6.82 (d, 1H), 7.23 (d, 2H), 7.35-7.47 (m, 3H), 7.51 (d, 2H), 7.59 (m, 1H), 7.69 (s, 1H), 7.70 (d, 1H).

### Example 52

### (2-Methyl-4-{[7-[4-fluorophenyl]-4-spirocyclohexyl-3,4-dihydro-2(1H)-isoquinolinyl]sulfonyl}phenoxy)acetic acid

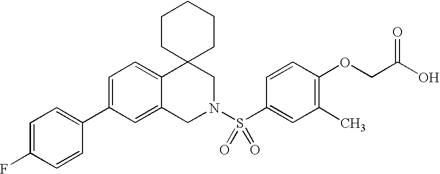

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 106.0 mg (0.192 mmol) of ethyl(2-methyl-4-{[4-spirocyclohexyl-7-(fluoro)phenyl-3,4-dihydro-2(1H)-isoquinolinyl]sulfonyl}phenoxy)acetate and 0.40 ml (0.40 mmol) of 1 M sodium hydroxide solution in 5 ml of ethanol are used to obtain, after extraction of the aqueous phase with ethyl acetate, drying over sodium sulfate and removal of the solvent under reduced pressure, 94 mg (86% of theory) of the desired product.

HPLC (method 1): Rₜ=5.53 min.

MS (ESIpos): 524 (M+H)⁺

¹H NMR (400 MHz, DMSO-d₆): δ=1.45-1.80 (m, 10H), 2.29 (s, 3H), 3.21 (s, 2H), 4.17 (s, 2H), 4.80 (s, 2H), 7.07 (d, 1H), 7.15 (d, 1H), 7.18 (d, 1H), 7.41 (s, 1H), 7.49 (d, 1H), 7.52 (d, 1H), 7.62-7.72 (m, 4H).

### Example 53

### (2-Methyl-4-{[4-spirocyclohexyl-7-[4-(trifluoromethyl)phenyl]-3,4-dihydro-2(1H)isoquinolinyl]sulfonyl}phenoxy)acetic acid

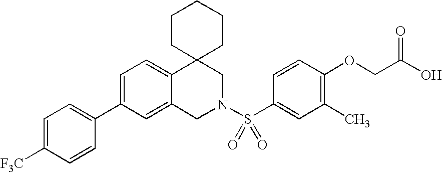

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 840.0 mg (1.396 mmol) of ethyl(2-methyl-4-{[4-spirocyclohexyl-7-(trifluoromethyl)phenyl-3,4-dihydro-2(1H)-isoquinolinyl]sulfonyl}phenoxy)acetate and 1.68 ml (1.68 mmol) of 1 M sodium hydroxide solution in 10 ml of ethanol are used to obtain, after extraction of the aqueous phase with ethyl acetate, drying over sodium sulfate, removal of the solvent under reduced pressure and purification by HPLC, 570 mg (71% of theory) of the desired product.

HPLC (method 1): Rₜ=5.69 min.

MS (ESIpos): 574 (M+H)⁺

¹H NMR (300 MHz, DMSO-d₆): δ=1.45-1.83 (m, 10H), 2.29 (s, 3H), 3.21 (s, 2H), 4.20 (s, 2H), 4.83 (s, 2H), 7.09 (d, 1H), 7.55 (s, 1H), 7.58 (s, 2H), 7.70 (d, 1H), 7.72 (s, 1H), 7.79 (d, 2H), 7.88 (d, 2H).

### Example 54

### (3-Methyl-{[4-spirocyclohexyl-7-[4-(trifluoromethyl)phenyl]-3,4-dihydro-2(1H)isoquinolinyl]sulfonyl}phenoxy)acetic acid

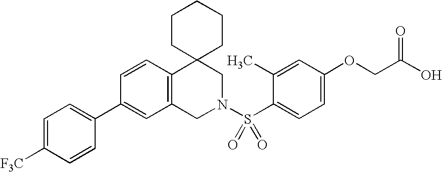

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 19.0 mg (0.032 mmol) of ethyl(3-methyl-4-{[4-spirocyclohexyl-7-(trifluoromethyl)phenyl-3,4-dihydro-2(1H)-isoquinolinyl]sulfonyl}phenoxy)acetate and 0.10 ml (0.10 mmol) of 1 M sodium hydroxide solution in 5 ml of ethanol are used to obtain 13 mg (72% of theory) of the desired product.

HPLC (method 1): Rₜ=5.75 min.

MS (ESIpos): 574 (M+H)⁺

¹H NMR (200 MHz, DMSO-d₆): δ=1.40-1.83 (m, 10H), 2.56 (s, 3H), 3.30 (s, 2H), 4.36 (s, 2H), 4.78 (s, 2H), 6.98 (d, 1H), 7.02 (s, 1H), 7.60 (s, 2H), 7.63 (m, 1H), 7.75-7.95 (m, 5H).

### Example 55

### (2,5-Dimethyl-4-{[4-spirocyclohexyl-7-[4-(trifluoromethyl)phenyl]-3,4-dihydro-2(1H)-isoquinolinyl]sulfonyl}phenoxy)acetic acid

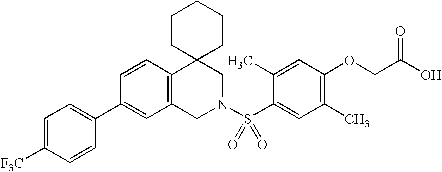

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 24.0 mg (0.039 mmol) of ethyl(2,6-dimethyl-4-{[4-spirocyclohexyl-7-(trifluoromethyl)phenyl-3,4-dihydro-2(1H)-isoquinolinyl]sulfonyl}phenoxy)acetate and 0.12 ml (0.12 mmol) of 1 M sodium hydroxide solution in 5 ml of ethanol are used to obtain 21 mg (92% of theory) of the desired product.

HPLC (method 1): Rₜ=5.88 min.

MS (ESIpos): 588 (M+H)⁺

¹H NMR (200 MHz, DMSO-d₆): δ=1.45-1.80 (m, 10H), 2.23 (s, 3H), 2.50 (s, 3H), 3.30 (s, 2H), 4.32 (s, 2H), 4.70 (s, 2H), 6.89 (s, 1H), 7.59 (s, 2H), 7.70 (s, 1H), 7.80 (d, 2H), 7.89 (d, 2H).

### Example 56

### (2,3-Dimethyl-4-{[4-spirocyclohexyl-7-[4-(trifluoromethyl)phenyl]-3,4-dihydro-2(1H)-isoquinolinyl]sulfonyl}phenoxy)acetic acid

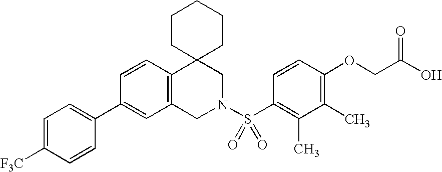

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 25.0 mg (0.041 mmol) of ethyl(2,3-dimethyl-4-{[4-spirocyclohexyl-7-(4-trifluoromethyl)phenyl-3,4-dihydro-2(1H)-isoquinolinyl]sulfonyl}phenoxy)acetate and 0.12 ml (0.24 mmol) of 2 M sodium hydroxide solution in 5 ml of ethanol are used to obtain 17 mg (71% of theory) of the desired product.

HPLC (method 1): Rₜ=5.90 min.

MS (ESIpos): 588 (M+H)⁺

¹H NMR (200 MHz, DMSO-d₆): δ=1.40-1.80 (m, 10H), 2.19 (s, 3H), 2.53 (s, 3H), 3.30 (s, 2H), 4.3.7 (s, 2H), 4.77 (s, 2H), 6.93 (d, 1H), 7.58 (m, 3H), 7.74-7.95 (m, 5H).

### Example 57

### (2-Methyl-4-{[4-(2-phenylethyl)-7-[4-(trifluoromethyl)phenyl]-3,4-dihydro-2(1H)isoquinolinyl]sulfonyl}phenoxy)acetic acid

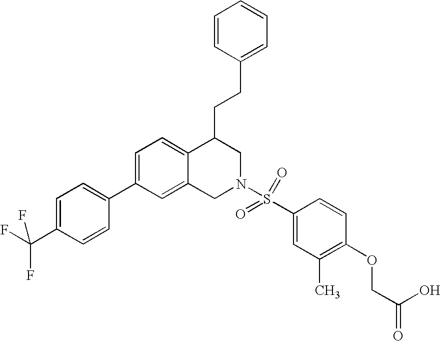

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid, 0.12 g (0.19 mmol) of ethyl(2-methyl-4-{[4-(2-phenylethyl)-7-[4-(trifluoromethyl)phenyl]-3,4-dihydro-2(1H)-isoquinolinyl]sulfonyl}phenoxy)acetate is stirred at room temperature with 2 ml of 2 N sodium hydroxide solution in 5 ml of ethanol for 3 hours. For workup, the mixture is concentrated under reduced pressure and the residue is taken up in diethyl ether and water. The phases are separated and the aqueous phase is made acidic using 1 N hydrochloric acid and extracted three times with ethyl acetate. The combined organic phases are combined and dried over sodium sulfate, filtered and freed of solvent under reduced pressure. The crude yield is purified using a 3 g silica gel cartridge (eluent: dichloromethane and 95:5 dichloromethane/ethyl acetate). The clean fractions are combined and freed of solvent under reduced pressure. 85 mg (73% of theory) of the desired product are obtained.

HPLC (method 1): Rₜ=5.73 min.

MS (DCI): 627 (M+NH₄)⁺

¹H NMR (300 MHz, DMSO-d₆): δ=1.87-1.99 (m, 2H), 2.26 (s, 3H), 2.60-2.84 (m, 2H), 2.87-2.97 (m, 2H), 3.68-3.79 (m, 1H), 3.97 (d, 1H), 4.54 (d, 1H), 4.82 (s, 2H), 7.02-7.09 (m, 1H), 7.13-7.36 (m, 6H), 7.49-7.59 (m, 2H), 7.63-7.72 (m, 2H), 7.78 (d, 2H), 7.86 (d, 2H), 12.98-13.26 (m, 1H).

### Example 58

### Ethyl(2-methyl-4-{[1,4,4-trimethyl-7-[4-(dimethylamino)phenyl]-3,4-dihydro-2(1H)-isoquinolinyl]sulfonyl}phenoxy)acetate

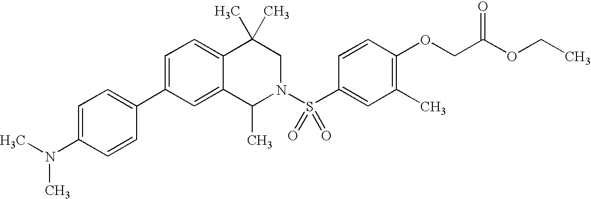

Analogously to the preparation of ethyl(2-methyl-4-{[1,4,4-trimethyl-7-(4-fluorophenyl)-3,4-dihydro-2(1H)-isoquinolinyl]sulfonyl}phenoxy)acetate (Example 19), 220 mg (0.380 mmol) of ethyl(2-methyl-{[7-{[(trifluoromethyl)sulfonyl]oxy}-1,4,4-trimethyl-3,4-dihydro-2(1H)-isoquinolinyl]sulfonyl}phenoxy)acetate, 125.3 mg (0.759 mmol) of 4-(dimethylamino)phenylboronic acid, 590 mg (1.898 mmol) of potassium phosphate trihydrate and 35 mg (0.030 mmol) of tetrakis(triphenylphosphine)palladium in 5 ml of toluene are used to obtain, after purification by HPLC, 53 mg (25% of theory) of the desired product.

HPLC (method 1): Rₜ=4.66 min.

MS (ESIpos): 551 (M+H)⁺

¹H NMR (400 MHz, CDCl₃): δ=1.17 (s, 3H), 1.29 (t, 3H), 1.31 (s, 3H), 1.35 (d, 3H), 2.31 (s, 3H), 2.99 (s, 6H), 3.18 (d, 1H), 3.51 (d, 1H), 4.27 (q, 2H), 4.69 (s, 2H), 5.17 (q, 1H), 6.72 (d, 1H), 6.79 (d, 2H), 7.17 (d, 1H), 7.30 (d, 1H), 7.38 (dd, 1H), 7.43 (d, 2H), 7.68 (s, 1H), 7.69 (d, 1H).

### Example 59

### Ethyl(2-methyl-4-{[1,4,4-trimethyl-7-[4-(trifluoromethoxy)phenyl]-3,4-dihydro-2(1H)-isoquinolinyl]sulfonyl}phenoxy)acetate

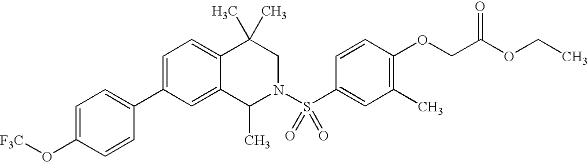

Analogously to the preparation of ethyl(2-methyl-4-{[1,4,4-trimethyl-7-(4-fluorophenyl)-3,4-dihydro-2(1H)-isoquinolinyl]sulfonyl}phenoxy)acetate (Example 19), 220 mg (0.380 mmol) of ethyl(2-methyl-{[7-{[(trifluoromethyl)sulfonyl]oxy}-1,4,4-trimethyl-3,4-dihydro-2(1H)-isoquinolinyl]sulfonyl}phenoxy)acetate, 195.4 mg (0.948 mmol) of 4-(trifluoromethoxy)phenylboronic acid, 590 mg (1.898 mmol) of potassium phosphate trihydrate and 17.4 mg (0.016 mmol) of tetrakis(triphenylphosphine)palladium in 5 ml of toluene are used to obtain, after purification by HPLC, 147 mg (65% of theory) of the desired product.

HPLC (method 1): Rₜ=5.89 min.

MS (ESIpos): 592 (M+H)⁺

¹H NMR (300 MHz, CDCl₃): δ=1.20 (s, 3H), 1.29 (t, 3H), 1.33 (s, 3H), 1.33 (d, 3H), 2.31 (s, 3H), 3.18 (d, 1H), 3.54 (d, 1H), 4.27 (q, 2H), 4.69 (s, 2H), 5.17 (q, 1H), 6.74 (d, 1H), 7.18 (d, 1H), 7.26 (d, 2H), 7.37 (d, 2H), 7.53 (d, 2H), 7.68 (s, 1H), 7.69 (d, 1H).

### Example 60

### (2-Methyl-4-{[1,4,4-trimethyl-7-[4-(dimethylamino)phenyl]-3,4-dihydro-2(1H)isoquinolinyl]sulfonyl}phenoxy)acetic acid

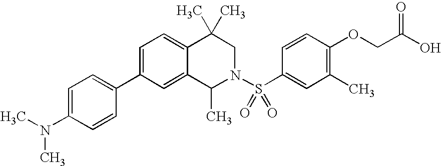

Analogously to the preparation of (2-methyl-4-{[1,4,4-trimethyl-7-(4-fluorophenyl)-3,4-dihydro-2(1H)-isoquinolinyl]sulfonyl}phenoxy)acetic acid (Example 34), 40.0 mg (0.073 mmol) of ethyl(2-methyl-4-{[1,4,4-trimethyl-7-[4-(dimethylamino)phenyl]-3,4-dihydro-2(1H)-isoquinolinyl]sulfonyl}phenoxy)acetate and 0.50 ml (1.50 mmol) of 2 M sodium hydroxide solution in 1 ml of ethanol are used to obtain 31 mg (82% of theory) of the desired product.

HPLC (method 1): Rₜ=4.86 min.

MS (ESIpos): 523 (M+H)⁺

¹H NMR (200 MHz, DMSO-d₆): δ=1.13 (s, 3H), 1.19 (d, 3H), 1.29 (s, 3H), 2.26 (s, 3H), 2.99 (s, 6H), 3.11 (d, 1H), 3.55 (d, 1H), 4.83 (s, 2H), 5.08 (q, 1H), 6.99-7.11 (m, 2H), 7.35-7.48 (m, 3H), 7.58 (d, 2H), 7.61-7.71 (m, 3H).

### Example 61

### (2-Methyl-4-{[1,4,4-trimethyl-7-[4-(trifluoromethoxy)phenyl]-3,4-dihydro-2(1H)isoquinolinyl]sulfonyl}phenoxy)acetic acid

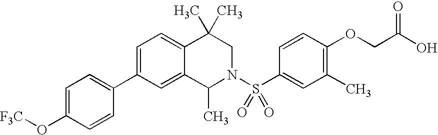

Analogously to the preparation of (2-methyl-4-{[1,4,4-trimethyl-7-(4-fluorophenyl)-3,4-dihydro-2(1H)-isoquinolinyl]sulfonyl}phenoxy)acetic-acid (Example 34), 130.0 mg (0.219 mmol) of ethyl(2-methyl-4-{[1,4,4-trimethyl-7-[4-(trifluoromethoxy)-phenyl]-3,4-dihydro-2(1H)-isoquinolinyl]sulfonyl}phenoxy)acetate and 1.0 ml (2.0 mmol) of 2 M sodium hydroxide solution in 2 ml of ethanol are used to obtain 106 mg (86% of theory) of the desired product.

HPLC (method 1): Rₜ=5.42 min.

MS (ESIpos): 564 (M+H)⁺

¹H NMR (200 MHz, DMSO-d₆): δ=1.15 (s, 3H), 1.20 (d, 3H), 1.31 (s, 3H), 2.26 (s, 3H), 3.13 (d, 1H), 3.58 (d, 1H), 4.79 (s, 2H), 5.11 (q, 1H), 7.00 (d, 1H), 7.39-7.57 (m, 5H), 7.67 (d, 1H), 7.69 (s, 1H), 7.77 (d, 2H).

### Example A

Cellular Transactivation Assay:

Test Principle:

A cellular assay is used to identify activators of the peroxisome proliferator-activated receptor delta (PPAR-delta).

Since mammalian cells contain various endogenous nuclear receptors which might complicate an unambiguous interpretation of the results, an established chimera system is used in which the ligand binding domain of the human PPARδ receptor is fused to the DNA binding domain of the yeast transcription factor GALA. The thus formed GALA-PPARδ chimera is co-transfected and stably expressed in CHO cells having a reporter construct.

Cloning:

The GAL4-PPARδ expression construct contains the ligand binding domain of PPARδ (amino acids 414-1326), which is PCR-amplified and cloned into the vector pcDNA3.1. This vector already contains the GALA DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) after activation and binding of GAL4-PPARδ.

Transactivation Assay (Luciferase Reporter):

CHO (chinese hamster ovary) cells are sown in CHO-A-SFM medium (GIBCO), supplemented by 2.5% fetal calf serum and 1% penicillin/streptomycin (GEBCO), at a cell density of 2×10³ cells per well in a 384-well plate (Greiner). The cells are cultivated at 37° C. for 48 h and then stimulated. To this end, the substances to be tested are taken up in the abovementioned medium and added to the cells. After a stimulation time of 24 hours, the luciferase activity is measured with the aid of a video camera. The relative light units measured give, as a function of the substance concentration, a sigmoidal stimulation curve. The EC₅₀ values are calculated with the aid of the computer program GraphPad PRISM (Version 3.02).

In this test, the inventive compounds of examples 18-22, 28, 30, 32-37, 41, 43, 45, 47, 50-57, 60 and 61 show an EC₅₀ value of from 1 to 100 nM.

### Example B

Descriptions of the Test for Finding Pharmacologically Active Substances which Increase HDL Cholesterol (HDL-C) Concentrations in the Serum of Transgenic Mice Transfected with the Human ApoA1 Gene (hApoA1) and/or Have an Effect on the Metabolic Syndrome of Adipose ob,ob Mice and Lower Their Blood Glucose Concentration:

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances, are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the last administration of substance (i.e. on day 8 after the start of the treatment), another blood sample is taken from each animal by puncture of the retroorbital venous plexus, to determine the same parameters. The blood samples are centrifuged and, after the serum has been obtained, cholesterol and TG are determined photometrically using an EPOS Analyzer 5060 (Eppendorf-Gerätebau, Netheler & Hinz GmbH, Hamburg). The said determinations are carried out using commercial enzyme tests (Boehringer Mannheim, Mannheim).

To determine the HDL-C, the non-HDL-C fraction is precipitated using 20% PEG 8000 in 0.2 M glycine buffer pH 10. From the supernatant, the cholesterol is determined UV-photometrically (BIO-TEK Instruments, USA) in a 96-well plate using a commercial reagent (Ecoline 25, Merck, Darmstadt).

Human mouse-ApoA1 is determined with a Sandwich ELISA method using a polyclonal anti-human-ApoA1 antibody and a monoclonal anti-human-ApoA1 antibody (Biodesign International, USA). Quantification is carried out UV-photometrically (BIO-TEK Instruments, USA) using peroxidase-coupled anti-mouse-IGG antibodies (KPL, USA) and peroxidase substrate (KPL, USA).

The effect of the test substances on the HDL-C concentration is determined by subtracting the value measured for the 1st blood sample (zero value) from the value measured for the 2nd blood sample (after the treatment). The mean of the differences of all HDL-C values of one group is determined and compared to the mean of the differences of the control group.

Statistical evaluation is carried out using Student's t-test, after the variances have been checked for homogeneity.

Substances which increase the HDL-C of the treated animals in a statistically significant (p<0.05) manner by at least 15%, compared to that of the control group, are considered to be pharmacologically effective.

To examine substances for their effect on a metabolic syndrome, animals having an insulin resistance and increased blood glucose levels are used. To this end, C57B1/6J Lep <ob> mice are treated using the same protocol as for the transgenic ApoA1 mice. The serum lipids are determined as described above. In these animals, serum glucose is additionally determined as a parameter for blood glucose. Serum glucose is determined enzymatically in an EPOS Analyzer 5060 (see above), using commercially available enzyme tests (Boehringer Mannheim).

A blood-glucose-lowering effect of the test substances is determined by subtracting the value measured for the 1st blood sample of an animal (zero value) from the value measured for the 2nd blood sample of the same animal (after the treatment). The mean of the differences of all serum glucose values of one group is determined and compared to the mean of the differences of the control group.

Statistical evaluation is carried out using Student's t-test, after the variances have been checked for homogeneity.

Substances which lower the serum glucose concentration of the treated animals in a statistically significant (p<0.05) manner by at least 10%, compared to that of the control group, are considered to be pharmacologically effective.

1. A compound of formula (I) 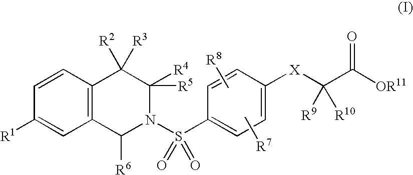
in which
X is O, S or CH₂,
R¹ is halogen, (C₁-C₆)-alkoxy, (C₂-C₆)-alkenyloxy, (C₃-C₇)-cycloalkoxy, optionally halogen-, (C₁-C₄)-alkyl-, trifluoromethyl- or (C₁-C₄)-alkoxy-substituted benzyloxy or
is (C₆-C₁₀)-aryl or 5- to 6-membered heteroaryl having up to three heteroatoms from the group of N, O and S, each of which may itself be mono- to trisubstituted, identically or differently, by substituents selected from the group of halogen, (C₁-C₆)-alkyl, (C₁-C₆)-alkoxy, trifluoromethyl, trifluoromethoxy, amino, mono- and di-(C₁-C₆)-alkylamino,
R² and R³ are the same or different and are each independently hydrogen or (C₁-C₆)-alkyl which is optionally substituted by phenyl, or, together with the carbon atom to which they are bonded, form a 3- to 7-membered, spiro-linked cycloalkyl ring,
R⁴ and R⁵ are the same or different and are each independently hydrogen or (C₁-C₆)-alkyl,
R⁶ is hydrogen or (C₁-C₆)-alkyl,
R⁷ is hydrogen or (C₁-C₆)-alkyl,
R⁸ is hydrogen, (C₁-C₆)-alkyl, (C₁-C₆)-alkoxy or halogen,
R⁹ and R¹⁰ are the same or different and are each independently hydrogen or (C₁-C₄)-alkyl, and
R¹¹ is hydrogen or is a hydrolyzable group which can be degraded to the corresponding carboxylic acid,
or a pharmaceutically acceptable salt thereof.

2. A compound of formula (I) as claimed in claim 1, in which
X is O or S,
R¹ is (C₂-C₄)-alkenyloxy, (C₅-C₆)-cycloalkoxy or is halogen or (C₁-C₄)-alkoxy, or
is 6-membered heteroaryl having up to two nitrogen atoms, or is phenyl or benzyloxy which may themselves each be mono- to disubstituted, identically or differently, by substituents selected from the group of fluorine, chlorine, (C₁-C₄)-alkyl, (C₁-C₄)-alkoxy, trifluoromethyl, trifluoromethoxy, amino, mono- and di-(C₁-C₄)-alkylamino,
R² and R³ are the same or different and are each independently hydrogen or are methyl or ethyl which may be substituted by phenyl, or, together with the carbon atom to which they are bonded, form a 4- to 6-membered, spiro-linked cycloalkyl ring,
R⁴ and R⁵ are each hydrogen,
R⁶ is hydrogen or methyl,
R⁷ is hydrogen or methyl,
R⁸ is hydrogen, (C₁-C₄)-alkyl, (C₁-C₄)-alkoxy, fluorine or chlorine,
R⁹ and R¹⁰ are the same or different and are each independently hydrogen or methyl, and
R¹¹ is hydrogen or (C₁-C₄)-alkyl,
or a pharmaceutically acceptable salt thereof.

3. A compound of formula (I) as claimed in claim 1, in which
X is O,
R¹ is pyridyl or is phenyl which may itself in each case be mono- or disubstituted, identically or differently, by substituents selected from the group of fluorine, chlorine, methyl, methoxy, trifluoromethyl, trifluoromethoxy, amino and dimethylamino,
R² is hydrogen or methyl,
R³ is methyl or phenethyl, or
R² and R³, together with the carbon atom to which they are bonded, form a spiro-linked cyclopentane or cyclohexane ring,
R⁴ and R⁵ are each hydrogen,
R⁶ is hydrogen or methyl,
R⁷ is hydrogen or methyl,
R⁸ is methyl,
R⁹ and R¹⁰ are each hydrogen, and
R¹¹ is ethoxy or is hydrogen,
or a pharmaceutically acceptable salt thereof.

4. A compound of formula (I) as claimed in any of claims 1 to 3,
in which
R⁴, R⁵, R⁹, R¹⁰ is hydrogen,
X is oxygen,
R⁸ is 2-methyl,
R² is hydrogen,
R³ is methyl or phenethyl, or
R² and R³ are each methyl, or, together with the carbon atom to which they are bonded, form a spiro-linked cyclopentane or cyclohexane ring, and
R¹, R⁶ and R⁷ are each as defined in claims 1 to 3,
or a pharmaceutically acceptable salt thereof.

5. A compound of formula (I) as claimed in claim 4,
in which
R² is hydrogen,
R³ is methyl or phenethyl, or
R² and R³ are each methyl, or, together with the carbon atom to which they are bonded, form a spiro-linked cyclopentane or cyclohexane ring,
or a pharmaceutically acceptable salt thereof.

6. A process for preparing a compound of formula (I) as defined in claim 1, characterized in that
a compound of formula (II) 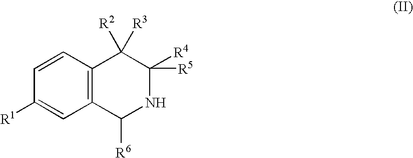
in which R¹, R², R³, R⁴, R⁵ and R⁶ are each as defined in claim 1
is converted initially, using a compound of formula (III) 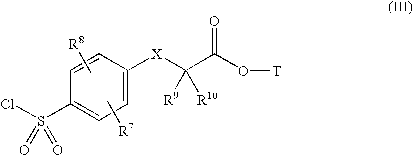
in which X, R⁷, R⁸, R⁹ and R¹⁰ are each as defined in claim 1 and
T is benzyl or (C₁-C₆)-alkyl,
in an inert solvent in the presence of a base, to a compound of formula (I-B) 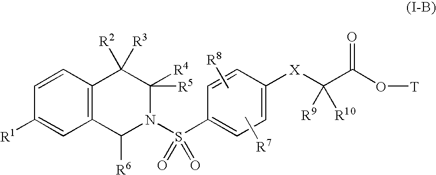
in which T, X, R¹, R², R³, R⁴, R⁵, R⁶, R⁷, R⁸, R⁹ and R¹⁰ are each as defined in claim 1,
this is then reacted with acid or base or, in the case that T is benzyl, also hydrogenolytically, to give the corresponding carboxylic acid of formula (I-C) 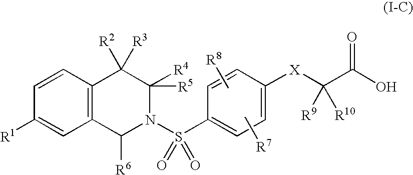
in which X, R¹, R², R³, R⁴, R⁵, R⁶, R⁷, R⁸, R⁹ and R¹⁰ are each as defined in claim 1,
and this carboxylic acid (I-C) is optionally modified further by known methods for esterification to give a compound of formula (I), or
(B) a compound of the general formula (IV) 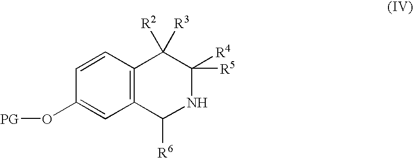
in which R², R³, R⁴, R⁵ and R⁶ are each as defined in claim 1 and
PG is a suitable hydroxyl protecting group is converted initially, using a compound of formula (III) in an inert solvent in the presence of a base to a compound of formula (V) 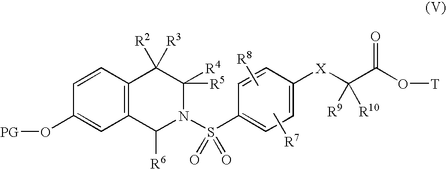
in which PG, T, X, R², R³, R⁴, R⁵, R⁶, R⁷, R⁸, R⁹ and R¹⁰ are each as defined in claim 1,
in the next reaction step the protecting group PG is removed by suitable methods to give a compound of formula (VI) 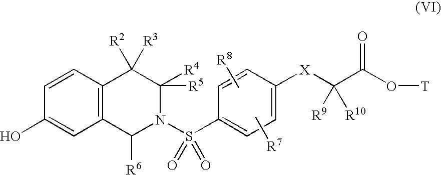
in which T, X, R², R³, R⁴, R⁵, R⁶, R⁷, R⁸, R⁹ and R¹⁰ are each as defined in claim 1,
and the compound of formula (VI) is then either
(B-1) reacted with a compound of formula (VII)
R¹²Z (VII)
in which
R¹² is (C₁-C₆)-alkyl, (C₂-C₆)-alkenyl, (C₃-C₇)-cycloalkyl or optionally halogen-, (C₁-C₄)-alkyl-, trifluoromethyl- or (C₁-C₄)-alkoxy-substituted benzyl, and
Z is a suitable leaving group,
in an inert solvent in the presence of a base to give a compound of formula (I-D) 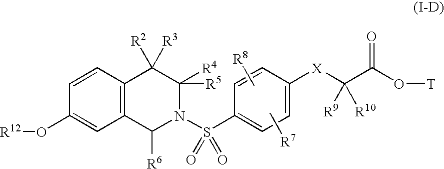
in which T, X, R², R³, R⁴, R⁵, R⁶, R⁷, R⁸, R⁹, R¹⁰ and R¹² are each as defined in claim 1,
and this is converted, using acid or base or, in the case that T is benzyl, also hydrogenolytically, to the corresponding carboxylic acid of formula (I-E) 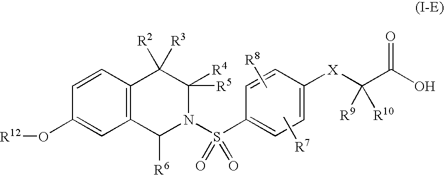
in which X, R², R³, R⁴, R⁵, R⁶, R⁷, R⁸, R⁹, R¹⁰ and R¹² are each as defined in claim 1, or
(B-2) initially converted, using trifluoromethanesulfonic anhydride in the presence of a base, to a compound of formula (VIII) 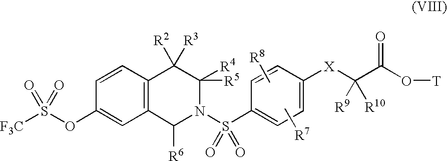
in which T, X, R², R³, R⁴, R⁵, R⁶, R⁷, R⁸, R⁹ and R¹⁰ are each as defined in claim 1,
and this is reacted in a coupling reaction with a compound of formula (IX) 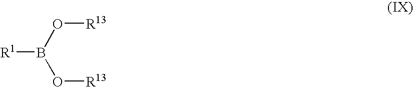
in which
R¹ is (C₆-C₁₀)-aryl or 5- to 6-membered heteroaryl having up to three heteroatoms from the group of N, O and S which may themselves be mono- to trisubstituted, identically or differently, by substituents selected from the group of halogen, (C₁-C₆)-alkyl, (C₁-C₆)-alkoxy, trifluoromethyl, trifluoromethoxy, amino, mono- and di-(C₁-C₆)-alkylamino, and
R¹³ is hydrogen or methyl or both radicals together form a CH₂CH₂ or C(CH₃)₂—C(CH₃)₂ bridge,
in an inert solvent in the presence of a suitable palladium catalyst and of a base to give the compound of formula (I-B).

7. (canceled)

8. A pharmaceutical composition comprising at least one compound of the formula (I) as defined in claim 1, and at least one inert, nontoxic, pharmaceutically suitable carrier, excipient, solvent, vehicle, emulsifier, or dispersant.

9. (canceled)

10. (canceled)

11. (canceled)

12. A method for the prevention and treatment of diseases, comprising administering to a patient in need thereof a therapeutically effective amount of a compound of claim 1.

13. The method of claim 12, wherein the disease is coronary heart disease, dyslipidemia, arteriosclerosis, myocardial infarction or restenosis after coronary angioplasty or stenting.

14. The process of claim 6, wherein the hydroxyl protecting group PG in formula (IV) in part (B) is a methyl or benzyl group.

15. The process of claim 6, wherein in the step of removing protecting group PG from the compound of formula (V) in part (B), boron tribromide is employed when PG is methyl, and PG is removed hydrogenolytically when PG is benzyl.
